(19) United States
(12) Patent Application Publication
(10) Pub. No.: US 2024/0000707 A1
(43) Pub. Date: Jan. 4, 2024

(54) INHALABLE DRY POWDER FORMULATIONS COMPRISING ANGIOGENESIS INHIBITORS

(71) Applicant: Lonza Bend Inc., Bend, OR (US)

(72) Inventors: Kimberly Shepard, Bend, OR (US); Michael Banks, New Rochelle, NY (US); David Vodak, Bend, OR (US)

(73) Assignee: Lonza Bend Inc., Bend, OR (US)

(21) Appl. No.: 18/253,190

(22) PCT Filed: Nov. 17, 2021
(86) PCT No.: PCT/US2021/059774

§ 371 Date: May 16, 2023

Related U.S. Application Data
(60) Provisional application No. 63/184,513, filed on May 5, 2021.
(60) Provisional application No. 63/174,926, filed on Apr. 14, 2021.
(60) Provisional application No. 63/151,499, filed on Feb. 19, 2021.
(60) Provisional application No. 63/115,255, filed on Nov. 18, 2020.

(51) Int. Cl. A61K 9/00; C07K 16/22; A61K 47/26; A61K 47/18; A61K 33/243; A61K 31/517; A61K 31/337; A61K 9/16

(52) U.S. Cl. CPC A61K 9/0075; C07K 16/22; A61K 47/26; A61K 47/183; A61K 33/243; A61K 31/517; A61K 31/337; A61K 9/1682; C07K 2317/24

(57) ABSTRACT

The present invention relates to inhalable dry powder formulations comprising one or more antibodies or one or more angiogenesis inhibiting active pharmaceutical ingredients, methods of manufacture of such compositions, e.g. via spray drying, as well as their local administration to the lung for use in the treatment, prevention and/or delay of progression of asthma, COPD, lung infections, cystic fibrosis, or lung cancer.

### FIELD OF THE INVENTION

The present invention relates to inhalable dry powder formulations comprising one or more antibodies or one or more angiogenesis inhibiting active pharmaceutical ingredients, methods of manufacture of such compositions, e.g. via spray drying, as well as their local administration to the lung for use in the treatment, prevention and/or delay of progression of asthma, COPD, lung infections, cystic fibrosis, or lung cancer.

All patent and non-patent references cited herein, are hereby incorporated by reference in their entirety.

### BACKGROUND

Angiogenesis is a biological process of generation of new blood vessels in a tissue or organ. Under normal physiological conditions, humans and animals undergo angiogenesis only in very specific restricted situations. For example, angiogenesis is normally observed in wound healing, fetal and embryonal development and formation of the corpus luteum, endometrium and placenta. It has been reported that new vessel growth is tightly controlled by angiogenic regulators and the switch of the angiogenesis phenotype depends on the net balance between up-regulation of angiogenic stimulators and down-regulation of angiogenic suppressors.

Angiogenesis is a normal process in growth and development, as well as in wound healing as described above. However, this is also a fundamental step in the transition of tumors from a small size with supply of substrate to larger size tumors and/or a growing metastasis, which needs its own vascular supply to continued growth.

Angiogenesis occurs stepwise as follows: vasodilation and increased permeability of preexisting vessels, decomposition of a basement membrane by protease produced by activated vascular endothelial cells, migration and proliferation of the vascular endothelial cells, tube formation of the vascular endothelial cells, formation of the basement membrane and encirclement of peripheral cells and finally the differentiation and maturation of blood vessels. Angiogenesis may be caused by various proliferation factors, cytokines, arachidonic acid metabolites, monobutyrin and the like with the proliferation factors considered most important. For angiogenesis to occur, pro-angiogenic factors must outweigh anti-angiogenic factors. Angiogenesis is closely related to various diseases particularly diabetic retinopathy, retinopathy of prematurity, macular degeneration, neovascular glaucoma, retinal vein occlusion, retinal artery occlusion, pterygium, rubeosis, corneal neovasculature, solid tumors, hemangioma, proliferation and transfer of tumors and the like. Vasculogenesis is the term used for spontaneous blood-vessel formation and intussusception is the term for new blood vessel formation by splitting off existing ones. Neovascularization allows tumor progression to ensue. With angiogenesis, the tumor becomes invasive locally and systemically. The modern clinical application of the principle “angiogenesis” can be divided into two main areas; anti-angiogenic therapies and pro-angiogenic therapies. Whereas anti-angiogenic therapies are trying to fight cancer and malignancies, the pro-angiogenic therapies are becoming more and more important in the search for new treatments for cardiovascular diseases.

The major mediator of tumor angiogenesis is vascular endothelial growth factor A (VEGF-A, also called VEGF). VEGF inhibitors hence act as angiogenesis inhibitors. VEGF signals through the VEGF receptor 2 (VEGFR-2), which mediates sprouting angiogenesis. VEGFR-2 is also called kinase-insert domain-containing receptor (KDR) in humans and fetal liver kinase 1 (fIK-1) in mice. VEGF is expressed in most types of human cancer, and increased expression in tumors is often associated with a less favorable prognosis. Induction of VEGF expression in tumors may be caused by factors such as hypoxia, low pH, inflammatory cytokines (e.g. interleukin-6), growth factors (e.g. basic fibroblast growth factor), sex hormones (both androgens and estrogens), and chemokines (e.g. stromal-cell-derived factor 1).

The binding of VEGF to VEGFR-2 activates a cascade of signaling events resulting in the up-regulation of genes mediating proliferation and migration of endothelial cells, promoting their survival as well as vascular permeability. The VEGFR-2 receptor dimerizes upon binding of VEGF, which is followed by intracellular activation of the PLCy-PKC-Raf kinase-MEK-mitogen-activated protein kinase (MAPK) pathway and subsequent initiation of DNA synthesis and cell growth, whereas activation of the phosphatidylinositol 3-kinase (PI3K)-Akt pathway leads to increased endothelial-cell survival. Activation of Src can lead to actin cytoskeleton changes and induction of cell migration. VEGF receptors are located on the endothelial-cell surface; however, intracellular (“intracrine”)-signaling VEGF receptors (VEGFR-2) may be present as well, and they are involved in promoting the survival of endothelial cells. The detailed structure of the intracellular VEGFR-2 in endothelial cells is not yet known, but it is shown as the full-length receptor that is normally bound to the cell surface. Binding of VEGF-C to VEGFR-3 mediates lymphangiogenesis. VEGF165 can bind to neuropilin (NRP) receptors, which can act as coreceptors with VEGFR-2 (horizontal arrow) to regulate angiogenesis.

VEGF promotes tumor angiogenesis through several mechanisms, including enhanced endothelial cell proliferation and survival; increased migration and invasion of endothelial cells; increased permeability of existing vessels, forming a lattice network for endothelial cell migration; and enhanced chemotaxis and homing of bone marrow derived vascular precursor cells (Niu et al., Curr Drug Targets (2010) 11(8): 1000-1017). In addition to having proangiogenic effects, VEGF has several important functions that are independent of vascular processes, including autocrine effects on tumor cell function (survival, migration, invasion), immune suppression, and homing of bone marrow progenitors to ‘prepare’ an organ for subsequent metastasis. Higher angiogenesis and VEGF expression have been detected in various human cancers including colorectal cancer, breast cancer, non-small cell lung cancer (NSCLC), renal cell cancer, glioblastoma multiforme and other tumors than corresponding nonmalignant normal tissue. Among patients with the highest levels of VEGF expression, survival was significantly worse than in patients with negative or lower levels of VEGF expression. VEGF levels were predictive of future metastases independently of nodal status and adjuvant chemotherapy, with a positive predictive value of 73%.

Lung cancers typically start in the cells lining the bronchi and parts of the lung such as the bronchioles or alveoli. Lung adenocarcinoma, a subset of NSCLC is the most common form of lung cancer (40%), and typically starts in the alveoli. Squamous cell carcinoma (aka epidermoid carcinoma) often begins in the bronchi near the middle of the lung. Large cell carcinomas may begin anywhere in the lung.

Metastasis in pulmonary parenchyma, i.e. in the terminal lung unit (TLU) in particular the alveolar and/or bronchoalveolar space and/or the small airways and/or the bronchioli and/or the alveolar cells including the alveolar macrophages and the pulmonary interstitium and pulmonary parenchyma.

Table 1 below provides an overview of currently approved VEGF inhibitors as well as the corresponding approved indications.

TABLE 1
Approved VEGF inhibitors

| INN | Brand Name | Approved indication |
|---|---|---|
| Ziv-aflibercept | Zaltrap ® | Colorectal cancer |
| Cabozantinib | Cabometyx ®, | Kidney/thyroid cancer |
| | Cometriq ® | |
| Pazopanib | Votrient ® | Kidney cancer/soft tissue |
| | | sarcoma |
| Sunitinib | Sutent ® | GI tumors |
| Axitinib | Inlyta ® | Kidney cancer |
| Lenvatinib | Lenvima ® | Thyroid cancer |
| Sorafenib | Nexavar ® | Thyroid, liver, kidney cancer |
| Regorafenib | Stivarga ® | Colorectal, liver cancer |
| Ponatinib | Iclusig ® | Leukemia |
| Vandetanib | Caprelsa ® | Thyroid cancer |
| Ramucirumab | Cyramza ® | gastric cancer |
| Ranibizumab | Lucentis ® | macular degeneration |
| Bevacizumab | Avastin ®/Mvasi ® | colon cancer, NSCLC |

Particular antibody VEGF inhibitors are bevacizumab, ramucirumab, and ranibizumab.

Bevacizumab (CAS: 216974-75-3; ChEMBL: ChEMBL1201583, DrugBank: DB00112; KEGG: D06409; UNII: 2S9ZZM9Q9V), sold under the brand name Avastin, is a medication used to treat a number of types of cancers. It is given by slow injection into a vein and used for colon cancer, lung cancer, glioblastoma, and renal-cell carcinoma. Bevacizumab is a monoclonal antibody that functions as an angiogenesis inhibitor. It works by slowing the growth of new blood vessels by inhibiting vascular endothelial growth factor A (VEGF-A), in other words anti-VEGF therapy. Bevacizumab was approved for medical use in the United States in 2004. It is on the World Health Organization's List of Essential Medicines.

A number of antibodies (mAb) are approved or are in development for use in the treatment of lung indications which are potentially suitable for administration via inhalation, particularly as dry powder.

Examples of antibodies suitable in treating and/or ameliorating asthma or COPD via dry powder inhalation are selected from:
Benralizumab (marketed as Fasenra®, target: IL-5),
Dupilumab (marketed as Dupixent®, target: IL-4),
Lebrikizumab (target: IL-13),
Mepolizumab (marketed as Nucala®, target: IL-5),
Omalizumab (marketed as Xolair®, target: IgE),
Reslizumab (marketed as Cinqair®/Cinqaero®, target: IL-5), and
Tralokinumab (target: IL-13).

Examples of antibodies suitable in treating and/or ameliorating lung infections via inhalation are selected from:
Oblitoxaximab (marketed as Anthim®, target: Bacillus anthracis),
Palivizumab (marketed as Synagis®, target: RSV),
Panobacumab (target: Pseudomonas aeruginosa), and
Raxibacumab (marketed as Abthrax®, target: Bacillus anthracis).

Examples of antibodies suitable in treating and/or ameliorating lung cancer, particularly NSCLC, via inhalation are selected from:
Atezolizumab (marketed as Tecentriq®, target PD-1/PDL-1),
Avelumab (marketed as Bavencio®, target PD-1/PDL-1),
Balstilimab (target PD-1/PDL-1),
Bevacizumab (marketed as Avastin®, target VEGF),
Camrelizumab (target PD-1/PDL-1),
Cemiplimab (marketed as Libtayo®, target PD-1/PDL-1),
Cetuximab (marketed as Erbitux®, target EGFR),
Dostarlimab (target PD-1/PDL-1),
Durvalumab (marketed as Imfinzi®, target PD-1/PDL-1),
Necitumumab (marketed as Portrazza®, target EGFR),
Nimotuzumab (marketed as Theraloc®, target EGFR),
Nivolumab (marketed as Opdivo®, target PD-1/PDL-1),
Panitumumab (marketed as Vectibix®, target EGFR),
Pembrolizumab (marketed as Keytruda®, target PD-1/PDL-1),
Prolgolimab (marketed as Forteca®, target PD-1/PDL-1),
Racotumomab (marketed as Vaxira®, target NeuGcGM3),
Ramucirumab (marketed as Cymraza®, target VEGF),
Ranibizumab (marketed as Lucentis®, target VEGF),
Retifanlimab (target PD-1/PDL-1),
Sintilimab (marketed as Tyvyt®, target PD-1/PDL-1),
Tislelizumab (target PD-1/PDL-1), and
Toripalimab (marketed as Tuoyi, target PD-1/PDL-1).

Particular antibodies suitable in treating and/or ameliorating lung indications, such as asthma, COPD, lung infections, and lung cancer, via dry powder inhalation are selected from the list of benralizumab, dupilumab, lebrikizumab, mepolizumab, omalizumab, reslizumab, tralokinumab, oblitoxaximab, palivizumab, panobacumab, raxibacumab, atezolizumab, avelumab, balstilimab, bevacizumab, camrelizumab, cemiplimab, cetuximab, dostarlimab, durvalumab, necitumumab, nimotuzumab, nivolumab, panitumumab, pembrolizumab, prolgolimab, racotumomab, ramucirumab, ranibizumab, retifanlimab, sintilimab, tislelizumab, and toripalimab.

Targeting the aerosol to conducting or peripheral airways can be accomplished by tailoring the particle size of the aerosol. Prediction of the actual site of deposition is difficult, since airway caliber and anatomy differ among people. Generally, it is accepted that a successful dry powder formulation for delivery to the lower/small airways and alveolar region of the lung must exhibit an aerodynamic diameter of approximately 1-5 μm (Bosquillon et al., J Controlled Release (2001) 70:329-339). For spherical particles, the aerodynamic diameter dₐ is defined as:

$${d}_{a} = {d}_{g} \sqrt{{ρ}_{p} / {ρ}_{0}}$$

where d_{g} is the geometric particle diameter, pp is the particle density in kg/m³ and ρ₀ is the standard particle density which is 1000 kg/m³.

Deposition in the alveolar region of the lung is preferable due to its immense surface area (100 m²) for drug absorption. For treatment of lung diseases, deposition in the deep lung is also critical for effectiveness. Aerosols with an aerodynamic diameter of 5-10 μm are mainly deposited in the large conducting airways and oropharyngeal region. Particles significantly larger than this will deposit in the throat, failing to reach the therapeutic area of the deep lung.

Both—formulation and manufacturing techniques—are used to maximize the fraction of dry powder particles for inhalation, such as e.g. spray dried particles, with aerodynamic diameter between 1 and 5 microns.

Devices to deliver dry powders to the lung are commonly called dry powder inhalers (DPI). A DPI is a device that delivers a metered unit dose of a medication to the lungs in the form of a dry powder. Several types of proprietary passive breathe-activated DPIs exist:
Single-dose (pre-metered) capsule inhalers consisting of a reusable device which needs to be loaded manually per inhalation with a capsule comprising a unit dose of inhalation powder (such as e.g. Aerolizer®, HandiHaler®, Neohaler®, PlastiApe®, Rotahaler®);
Multi-dose (pre-metered) inhalers comprising blister strips or cartridges with metered doses of inhalation powder which are operated semi-automatically (such as e.g. Diskus®, Ellipta®, Acu-Breathe®);
Bulk reservoir (device-metered) inhalers wherein individual doses are isolated by volumetric measurement from a powder reservoir inside the inhaler per loading/actuation cycle (such as Turbuhaler®, RespiClick®).

Critically, the medicine is self-administered by the patient, in a home setting. For capsule inhalers, a size 2 or 3 capsule filled with powder is loaded into a capsule-based dry powder inhaler, such as a passive PlastiApe® or Neohaler® device. The device's buttons are pressed to puncture the capsule, then the powder is inhaled through the mouthpiece to deliver the dose. One or multiple doses may be administered in consecutive capsules.

Administration of an antibody or angiogenesis inhibitor in powder form via inhalation according to the present invention solves the following unmet needs and technical problems:
Reduced dose: In the state of the art lung cancer treatment, angiogenesis inhibitors must be delivered by IV infusion due to the high dose required to reach the target tissue (the lung). The IV dose is distributed among all the tissues of the body, meaning only a fraction of that reaches the lung. By delivering the angiogenesis inhibitor locally to the lung, the drug is not distributed to other tissues in the body, thus the total dose can be reduced.
Reduced adverse effects: Serious side effects are associated with the high systemic exposure of angiogenesis inhibitor therapy via IV infusion, including severe bleeding and liver injury. By delivering the angiogenesis inhibitor locally to the lung, significant systemic exposure can be avoided, reducing the risk of adverse events in non-lung organs. Reduction of systemic exposure is particularly effective for lung administration of antibody drugs, as their large molecular size prevents nearly all systemic absorption.
Improved therapy management: IV infusions must be conducted in-clinic, leading to poor patient compliance, high cost, and inflexibility in dosing regimen. An inhalable formulation enables self-administration by the patient, and the possibility for daily dosing, rather than every 2-3 weeks.
Reduced peak concentration: The possibility for daily dosing via self-administered inhaled powder also helps to reduce side effects by reducing peak concentrations in the body which typically occur with IV infusion.

### SUMMARY OF THE INVENTION

In a first aspect, the invention provides a dry powder formulation comprising spray-dried solid dispersions (SDD) of an antibody or an angiogenesis inhibitor suitable for administration via inhalation.

A further aspect of the invention provides a dry powder formulation comprising SDDs of an antibody or an angiogenesis inhibitor and further a small molecular API suitable for administration via inhalation.

Another aspect of the invention relates to capsules, blister packs or blister strips comprising a dry powder formulation comprising SDDs of an antibody or an angiogenesis inhibitor suitable for administration via inhalation.

A further aspect of the invention is to provide a method for local delivery of an antibody or an angiogenesis inhibitor to lung tissue via inhalation.

Another aspect of the invention is to provide a method of treatment, prevention and/or delay of progression of lung indications, such as asthma, COPD, lung infections, cystic fibrosis, and lung cancer, comprising the administration via inhalation of a dry powder formulation comprising SDDs of an antibody or an angiogenesis inhibitor, which can optionally be self-administered by the patient.

### BRIEF DESCRIPTION OF THE FIGURES

FIG. 1: PXRD of as-received L-leucine, as-received trehalose dihydrate, and SDD formulations of Example 1 (10/70/20 bevacizumab/trehalose/L-leucine) & Example 3 (20/60/20 bevacizumab/trehalose/L-leucine).

FIG. 2: SEM image of SDD 10/70/20 bevacizumab/trehalose/L-leucine of Example 1.

FIG. 3: Next Generation Impactor results for SDD formulations of Example 1 (10/70/20 bevacizumab/trehalose/L-leucine) & Example 3 (20/60/20 bevacizumab/trehalose/L-leucine).

FIG. 4: Anti-VEGF activity assay of SDD formulation 10/70/20 bevacizumab/trehalose/L-leucine of Example 1 and bevacizumab solution control.

FIG. 5: SEM image of SDD 10/70/20 bevacizumab/trehalose/L-leucine of Example 2.

FIG. 6: SEM image of SDD 20/60/20 bevacizumab/trehalose/L-leucine of Example 3.

FIG. 7: Anti-VEGF activity assay of SDD formulation 20/60/20 bevacizumab/trehalose/L-leucine of Example 3 and bevacizumab solution control.

FIG. 8: PXRD of SDD formulation 40/40/20 bevacizumab/trehalose/L-leucine of Example 4 showing signature peaks of spray dried crystalline leucine.

FIG. 9: SEM image of SDD 40/40/20 bevacizumab/trehalose/L-leucine of Example 4.

FIG. 10: Photo of bevacizumab solution before spray drying (left) and reconstituted SDD formulation 40/40/20 bevacizumab/trehalose/L-leucine of Example 4 in buffer.

FIG. 11: Particle Size Distribution by laser light scattering of SDD formulation 40/40/20 bevacizumab/trehalose/L-leucine of Example 4.

FIG. 12: Next Generation Impactor results for SDD formulation 40/40/20 bevacizumab/trehalose/L-leucine of Example 4. Stage 1>8.1 μm, Stage 2: 4.5-8.1 μm; Stage 3: 2.8-4.5 μm; Stage 4: 1.7-2.8 μm; Stage 5: 0.9-1.7 μm; Stage 6: 0.6-0.9 μm; Stage 7: 0.3-0.6 μm; MOC: <0.3 μm

FIG. 13: Anti-VEGF activity assay of SDD formulation 40/40/20 bevacizumab/trehalose/L-leucine of Example 4 and bevacizumab solution control.

FIG. 14: Normalized lung weight at the conclusion of the in vivo primary efficacy study according to Example 5. Horizontal lines indicate mean of the data.

FIG. 15: Normalized lung weight at the conclusion of the in vivo maintenance study according to Example 5. Horizontal lines indicate mean of the data.

FIG. 16: Survival of rats during in vivo maintenance study according to Example 5.

FIG. 17. SEM image of dual-API SDDs of Cisplatin:Bevacizumab (5 wt % cisplatin/20 wt % bevacizumab/55 wt % trehalose/20 wt % L-leucine) of Example 6.

FIG. 18. SEM image of dual-API SDDs of Cisplatin:Bevacizumab (10 wt % cisplatin/20 wt % bevacizumab/50 wt % trehalose/20 wt % L-leucine) of Example 7.

FIG. 19. SEM image of co-sprayed mono-API SDDs Erlotinib:Bevacizumab (co-spray ratio 1:2) (80 wt % erlotinib/20 wt % L-Leucine and 40 wt % bevacizumab/40 wt % trehalose/20 wt % L-leucine) of Example 8.

FIG. 20. SEM image of co-sprayed mono-API SDDs Erlotinib:Bevacizumab (co-spray ratio 1:1) (80 wt % erlotinib/20 wt % L-Leucine and 40 wt % bevacizumab/40 wt % trehalose/20 wt % L-leucine) of Example 9.

FIG. 21. SEM image of co-sprayed mono-API SDDs Paclitaxel:Bevacizumab (co-spray ratio 1:5) (80 wt % paclitaxel/20 wt % L-Leucine and 40 wt % bevacizumab/40 wt % trehalose/20 wt % L-leucine) of Example 10.

FIG. 22. SEM image of co-sprayed mono-API SDDs Paclitaxel:Bevacizumab (co-spray ratio 1:2) (80 wt % paclitaxel/20 wt % L-Leucine and 40 wt % bevacizumab/40 wt % trehalose/20 wt % L-leucine) of Example 11.

FIG. 23. SEM image of co-sprayed mono-API SDDs Paclitaxel:Bevacizumab (co-spray ratio 1:1) (80 wt % paclitaxel/20 wt % L-Leucine and 40 wt % bevacizumab/40 wt % trehalose/20 wt % L-leucine) of Example 12.

FIG. 24. SEM image of co-sprayed mono-API SDDs Paclitaxel:Bevacizumab (co-spray ratio 2:1) (80 wt % paclitaxel/20 wt % L-Leucine and 40 wt % bevacizumab/40 wt % trehalose/20 wt % L-leucine) of Example 13.

FIG. 25. SEM image of co-sprayed mono-API SDDs Paclitaxel:Bevacizumab (co-spray ratio 5:1) (80 wt % paclitaxel/20 wt % L-Leucine and 40 wt % bevacizumab/40 wt % trehalose/20 wt % L-leucine) of Example 14.

FIG. 26. SEM image of co-sprayed mono-API SDDs Cisplatin:Bevacizumab (co-spray ratio 2:1) (10 wt % cisplatin/70 wt % trehalose/20 wt % L-Leucine and 40 wt % bevacizumab/40 wt % trehalose/20 wt % L-leucine) of Example 15.

FIG. 27. SEM image of co-sprayed mono-API SDDs Cisplatin:Bevacizumab (co-spray ratio 1:1) (10 wt % cisplatin/70 wt % trehalose/20 wt % L-Leucine and 40 wt % bevacizumab/40 wt % trehalose/20 wt % L-leucine) of Example 16

FIG. 28: PXRD of bevacizumab SDDs from examples 17, 18 and 19.

FIG. 29: SEM image of SDD 40/40/20 bevacizumab/mannitol/L-leucine SDD of Example 17.

FIG. 30: SEM image of SDD 40/55/5 bevacizumab/trehalose/L-leucine SDD of Example 18.

FIG. 31: SEM image of SDD 40/40/20 bevacizumab/trehalose/L-arginine SDD of Example 19.

FIG. 32: PXRD of 40/40/20 bevacizumab/trehalose/trileucine SDD of Example 20.

FIG. 33: SEM image of 40/40/20 bevacizumab/trehalose/trileucine SDD of Example 20.

FIG. 34: PXRD of 40/35/20/5 bevacizumab/trehalose/trileucine/histidine SDD of Example 21.

FIG. 35: SEM image of 25/25/50 bevacizumab/trehalose/L-leucine SDD of Example 22.

FIG. 36: PXRD of 4/85.5/10/0.5 bevacizumab/trehalose/L-leucine/phosphate SDD of Example 23.

FIG. 37: SEM image of 40/44.9/10/5.1 bevacizumab/trehalose/L-leucine/phosphate SDD of Example 24.

FIG. 38: SEM image of 40/40/20 bevacizumab/trehalose/L-leucine SDD of Example 25.

FIG. 39: SEM image of 40/40/20 bevacizumab/trehalose/L-leucine SDD of Example 26.

### DETAILED DESCRIPTION OF THE INVENTION

The term “active pharmaceutical ingredient” or “API” refers to a drug substance, formulated in a pharmaceutical formulation or drug product, intended to furnish pharmacological activity or to otherwise have direct effect in the diagnosis, cure, mitigation, treatment or prevention of disease, or to have direct effect in restoring, correcting or modifying physiological functions in a patient.
The term “antibody” or “mAb” or “monoclonal antibody” denotes an active pharmaceutical ingredient (API) selected from the list of benralizumab, dupilumab, lebrikizumab, mepolizumab, omalizumab, reslizumab, tralokinumab, oblitoxaximab, palivizumab, panobacumab, raxibacumab, atezolizumab, avelumab, balstilimab, bevacizumab, camrelizumab, cemiplimab, cetuximab, dostarlimab, durvalumab, necitumumab, nimotuzumab, nivolumab, panitumumab, pembrolizumab, prolgolimab, racotumomab, ramucirumab, ranibizumab, retifanlimab, sintilimab, tislelizumab, and toripalimab. Particular antibody is bevacizumab.

The term “angiogenesis inhibitor” denotes an active pharmaceutical ingredient that inhibits angiogenesis. Particular angiogenesis inhibitors are VEGF inhibitors. Particular angiogenesis inhibitor is bevacizumab.

The term “VEGF inhibitor” denotes an active pharmaceutical ingredient that inhibits VEGF. Particular VEGF inhibitors are aflibercept, axitinib, bevacizumab, cabozantinib, lenvatinib, pazopanib, ponatinib, ramucirumab, ranibizumab, regorafenib, sorafenib, sunitinib, and vandetanib. Particular VEGF inhibitors are bevacizumab, ramucirumab, and ranibizumab. Most particular VEGF inhibitor is bevacizumab

The terms “formulation” and “dry powder formulation” are used synonymously herein to denote a medicinal product or dosage form suitable for administration to a patient comprising one or more active pharmaceutical ingredients and one or more pharmaceutically acceptable excipients. Particularly, the formulation is solid. Particularly, the formulation is a solid dispersion. More particularly, the formulation is a spray dried solid dispersion (SDD). Particularly, the formulation is suitable for administration to a patient via inhalation. Particularly, the formulation is a dry powder comprising SDD particles.

The term “solid dispersion” is defined as a dispersion of at least two different components, i.e. one or more active pharmaceutical ingredients and an inert carrier or matrix, in a solid state. Preferably the components of the solid dispersion form an eutectic mixture. Preferably, the carrier or matrix is hydrophilic. Preferably, the carrier or matrix is amorphous. The active pharmaceutical ingredient(s) can be dispersed molecularly or be present in clusters, e.g. amorphous particles or crystalline particles.

The term “SDD” refers to a spray dried solid dispersion in which multiple components are dissolved in a common solvent, then atomized into a spray dryer, where the solvent is rapidly removed by a hot drying gas. The resulting dried powder is referred to as the SDD. In an SDD, the components may be molecularly dispersed, or the components may be phase separated within a single SDD particle into submicron domains.

The term “fixed-dose combination” (FDC) refers to a formulation wherein two or more active pharmaceutical ingredients are combined in one single dosage form at predetermined dosages. Particular examples of fixed-dose combinations according to the invention are dual-API SDDs and co-sprayed mono-API SDDs.

The term “dual-API SDD” refers to a formulation which is a fixed-dose combination comprising one single type of SDDs comprising a small molecular API and an angiogenesis inhibitor, particularly wherein the majority of SDD particles comprises both active ingredients (small molecular API and angiogenesis inhibitor), more particularly wherein each SDD particle comprises both active ingredients (small molecular API and antibody or angiogenesis inhibitor). Preferably, the dual-API SDDs are prepared by spray drying of one single spray solution comprising a small molecular API and an angiogenesis inhibitor.

The term “co-sprayed mono-API SDDs” refers to a formulation which is a fixed-dose combination comprising two types of co-sprayed mono-API SDDs, i.e. wherein the first type of mono-API SDDs comprises a small molecular API and wherein the second type of mono-API SDDs comprises an antibody or angiogenesis inhibitor, particularly no SDD particle comprises both active ingredients (small molecular API and angiogenesis inhibitor). Preferably, the co-sprayed mono-API SDDs are prepared by co-spray drying of two spray solutions, wherein the first spray solution comprises a small molecular API and wherein the second spray solution comprises an antibody or angiogenesis inhibitor.

The term “SEM” refers to the analytical method of scanning electron microscopy.

The term “DSC” denotes the analytical method of Differential Scanning Calorimetry. DSC thermograms were recorded using a Mettler-Toledo™ differential scanning calorimeter DSC3+, calibrated with indium as a standard. For the measurements, approximately 2-6 mg of sample were placed in aluminum pans, accurately weighed and hermetically closed with perforation lids. Prior to measurement, the lids were automatically pierced resulting in approx. 1.5 mm pin holes. The samples were then heated under a flow of nitrogen of about 50 mL/min in ADSC mode from 0° C. to 170° C. using heating rates of 2.5 K/min, with a 1.5 K amplitude modulation and 60 s period. The Tg was analyzed using STARe software.

The term “onset” denotes the intersection point of the baseline before transition and the interflection tangent.

The term “glass transition temperature” (Tg) denotes the temperature above which a glassy amorphous solid becomes rubbery.

The term “ambient condition” refers to a temperature of about 20° C.±5° C. and an atmospheric pressure of about 101.3 kPa±10 kPa.

The term “average moisture content” refers to the amount of water in a sample as determined using Karl-Fischer (KF) titration.

The terms “XRPD” and “PXRD” denote the analytical method of X-Ray Powder Diffraction which is used to determine the presence and identity of crystalline components in the solid material. XRPD patterns were recorded at ambient conditions in transmission geometry with a Rigaku MiniFlex 600 X-Ray diffractometer operating with a copper anode (K_{α1}=1.5060 Angstroms; K_{α2}=1.55549 Angstroms) generator set at 45 kV and 15 mA, in 2-theta range 3° to 40°, scanned at a rate of 2.5° 26 per minute in continuous scanning mode, and using a D/teX Ultra high speed detector. The samples were prepared and analyzed without further processing (e.g. grinding or sieving) of the substance. The relative XRPD peak intensity is dependent upon many factors such as structure factor, temperature factor, crystallinity, polarization factor, multiplicity, and Lorentz factor. Relative intensities may vary considerably from one measurement to another due to preferred orientation effects.

The abbreviation “FWHM” denotes the full width at half maximum, which is a width of a peak (e.g. appearing in a spectrum, particularly in an XRPD pattern) at its half height.

The term “sharp Bragg diffraction peak” in connection with X-ray diffraction patterns denotes a peak which is observed if Bragg's law of diffraction is fulfilled. Generally, the FWHM of a sharp Bragg diffraction peak is less than 0.5° 2-theta.

The term “amorphous form” denotes a solid material which does not possess a distinguishable crystal lattice and the molecular arrangement of molecules lacks a long-range order. In particular, amorphous denotes a material that does not show a sharp Bragg diffraction peak. Bragg's law describes the diffraction of crystalline material with the equation “2d*25 sin(theta)=n lambda”, wherein “d” denotes perpendicular distance (in Angstroms) between pairs of adjacent planes in a crystal (“d-spacing”), “theta” denotes the Bragg angle, “lambda” denotes the wavelength and “n” is an integer. When Bragg's law is fulfilled, the reflected beams are in phase and interfere constructively so that Bragg diffraction peaks are observed in the Xray diffraction pattern. At angles of incidence other than the Bragg angle, reflected beams are out of phase and destructive interference or cancellation occurs. Amorphous material does not satisfy Bragg's law and no sharp Bragg diffraction peaks are observed in the X-ray diffraction pattern. The XRPD pattern of an amorphous material is further characterized by one or more amorphous halos.

The term “amorphous halo” in connection with X-ray diffraction patterns denotes an approximately bell-shaped diffraction maximum in the X-ray powder diffraction pattern of an amorphous material. The FWHM of an amorphous halo is on principle larger than the FWHM of the peak of crystalline material.

The term “PSD” refers to the particle size distribution of a powder as measured by laser light scattering or using a cascade impactor, such as a Next Generation Impactor.

In this application particle sizes as determined by laser light scattering are expressed as volume mean diameters and particle sizes as determined by cascade impactor are expressed as mass mean diameters.

In this application particle sizes by laser light scattering were obtained using a Malvern Mastersizer 3000 (settings: Aero S disperser, Fraunhofer approximation, 2 psi dispersion pressure).

The term “equivalent spherical diameter” (or ESD) of a non-spherical object, e.g. an irregularly-shaped particle, is the diameter of a sphere of equivalent volume.

The terms “d50 value” and “median aerodynamic diameter” (MAD) are used synonymously herein and denote the average particle size, i.e. the average equivalent spherical diameter, which is defined as the diameter where 50% (either by mass or by volume, depending on the method used) of the particles of the ensemble have a larger equivalent spherical diameter, and the other 50% have a smaller equivalent spherical diameter.

The term “mass median aerodynamic diameter” (MMAD) is the d50 value by mass.

The term “d90 value” denotes the average particle size, i.e. the average equivalent spherical diameter, where 90% (either by mass or by volume, depending on the method used) of the particles of the ensemble have a smaller equivalent spherical diameter.

The term “d10 value” denotes the average particle size, i.e. the average equivalent spherical diameter, where 10% (either by mass or by volume, depending on the method used) of the particles of the ensemble have a smaller equivalent spherical diameter.

A “Next Generation Impactor” (NGI) is a high performance cascade impactor with seven stages. The NGI is the standard used in USP guidelines for testing of the aerodynamic powder properties of an inhalation powder. In this application, a Next Generation Impactor (NGI) (Model 170, MSP Corp.) with a high-resistance 4-kPa Plastiape RS01 monodose dry powder inhaler was employed. 10 mg of specimen were hand-filled into size 3 Vcaps Plus capsules (Capsugel). A pre-separator containing 10 mL of PBS was used upstream of the NGI. The test was operated at 65 L/min for 4.0 seconds. The contents of Pans 2 through 7 were dissolved in 5 mL of pH 7.4 PBS; Pans 1 and 8 were dissolved in 10 mL of pH 7.4 PBS. The bevacizumab content in the SDDs was measured using an absorbance technique, using ultraviolet (UV) probes (Pion Rainbow MicroDISS Profiler™, 20-mm path length). A known quantity of SDD was dissolved in pH 7.4 PBS, and multiple dilutions were prepared. Standards were prepared using as-received bevacizumab stock. The second-derivative of the absorbance over 276 to 284 nm was used to quantify the bevacizumab (trehalose and L-leucine do not absorb at this wavelength range).

A “Fast Scanning Impactor” or “Fast Screening Impactor” (FSI) separates an emitted dose into and measures Coarse Particle Mass (CPM) and Fine Particle Mass (FPM) at a standard or custom cut point. In present application, the cut point between CPM and FPM is set at 5 μm. In this application, a FSI was employed together with a Plastiape RS01 monodose dry powder inhaler, wherein 10 mg of specimen were hand-filled into size 3 Vcaps Plus capsules (Capsugel).

An “Aerodynamic Particle Sizer” (APS) by TSI Inc., Minnesota. was used to measure the aerodynamic diameter of particles from 0.5 to 20 micrometers. Time-of-flight aerodynamic sizing determines the particle's behavior while airborne and is unaffected by index of refraction or Mie scattering.

The term “fine particle fraction” (FPF) as used herein is defined as the fraction of particles of a respirable dose, i.e. the fraction of particles of an emitted dose that are smaller than the particle size that is considered the upper particle size limit to be respirable and in vivo deposited in the lung, i.e. the fraction of particles of an emitted dose smaller than 5.0 μm aerodynamic diameter. FPF is used as a performance characteristic of a formulation for inhalation or of an inhalation device regarding lung deposition (LD), e.g. for mechanistic modeling, in vitro-in vivo correlation, and to make estimations of clinical relevance of an inhaled product. FPF is typically measured using in vitro deposition techniques, such as impactors, such as e.g. a Next Generation Impactor (NGI) or Fast Scanning Impactor (FSI). For NGI the FPF is normalized by the emitted dose (i.e. fill mass minus masses retained in capsule and in device). For FSI the FPF is normalized by the fill mass only, ignoring the masses retained in capsule and in device.

The terms “very fine particle fraction” (vPFP) and “extra fine particle fraction” (eFPF) are used synonymously herein and denote the fraction of particles of an emitted dose smaller than 2.0 μm aerodynamic diameter.

The “fine particle dose” (FPD) corresponds to the mass of particles with aerodynamic diameter below 5 μm within the total emitted dose. In present application, the FPD was measured using a Fast Scanning Impactor, whereby the mass fraction of particles with an aerodynamic diameter of <5 μm were quantified gravimetrically. The FPD was normalized by the capsule fill mass (10 mg nominal).

The “geometric standard deviation” (GSD) measures the dispersion of particle diameter and is defined as the ratio of the median diameter to the diameter at ±1 sd (a) from the median diameter. In a cumulative distribution plot of the aerodynamic diameter and mass of particles, the GSD is calculated as the ratio of the median diameter to the diameter at 15.9% of the probability scale, or the ratio of the diameter at 84.1% on the probability scale to the median diameter. Aerosols with a GSD>1.22 are considered polydisperse. Most therapeutic aerosols are polydisperse and have GSDs in the range of 2-3.

The term “aqueous solubility” refers to the saturation concentration of a solute in water at ambient conditions (25° C., 1 atm) at neutral pH at equilibrium.

The term “COPD” refers to chronic obstructive pulmonary disease which is a type of obstructive lung disease characterized by long-term breathing problems and poor airflow.

Unless otherwise indicated, all concentrations provided herein are by weight (wt %).

The overall sum of concentrations of ingredients of the formulation does not exceed 100 wt %.

The term “about” used in connection with a numerical value indicates that the actual value can be within a range of ±20% of the specified numerical value, particularly within a range of ±10% of the specified numerical value, more particularly within a range of ±5% of the specified numerical value. The term “about” encompasses all values within a range of ±20%, particularly ±10%, more particularly ±5%, of the specified numerical value In one aspect, the invention provides a dry powder formulation suitable for administration via inhalation comprising one or more antibodies or one or more angiogenesis inhibitors.

In one aspect, the invention provides a dry powder formulation suitable for administration via inhalation comprising one or more antibodies or one or more angiogenesis inhibitors and a stabilizer.

In one aspect, the invention provides a dry powder formulation suitable for administration via inhalation comprising one or more antibodies or one or more angiogenesis inhibitors, a stabilizer and a dispersant.

In one aspect, the invention provides a dry powder formulation suitable for administration via inhalation comprising a spray-dried solid dispersion (SDD) of one or more antibodies or one or more angiogenesis inhibitors.

In one aspect, the invention provides a dry powder formulation suitable for administration via inhalation comprising a spray-dried solid dispersion (SDD) of one or more antibodies or one or more angiogenesis inhibitors and a stabilizer.

In one aspect, the invention provides a dry powder formulation suitable for administration via inhalation comprising a spray-dried solid dispersion (SDD) of one or more antibodies or one or more angiogenesis inhibitors, a stabilizer and a dispersant.

In one embodiment of the invention, the formulation comprises a spray-dried solid dispersion (SDD).

In one embodiment of the invention, the formulation is a spray-dried solid dispersion (SDD).

In one embodiment of the invention, the formulation comprises one or more antibodies.

In one embodiment of the invention, the antibody is selected from the list of benralizumab, dupilumab, lebrikizumab, mepolizumab, omalizumab, reslizumab, tralokinumab, oblitoxaximab, palivizumab, panobacumab, raxibacumab, atezolizumab, avelumab, balstilimab, bevacizumab, camrelizumab, cemiplimab, cetuximab, dostarlimab, durvalumab, necitumumab, nimotuzumab, nivolumab, panitumumab, pembrolizumab, prolgolimab, racotumomab, ramucirumab, ranibizumab, retifanlimab, sintilimab, tislelizumab, and toripalimab.

In one embodiment of the invention, the formulation comprises one or more angiogenesis inhibitors.

In one embodiment of the invention, the formulation comprises one or more VEGF inhibitors.

In one embodiment of the invention, the angiogenesis inhibitor is a VEGF inhibitor.

In one embodiment of the invention, the angiogenesis inhibitor is selected from the list of aflibercept, axitinib, bevacizumab, cabozantinib, lenvatinib, pazopanib, ponatinib, ramucirumab, ranibizumab, regorafenib, sorafenib, sunitinib, and vandetanib.

In one embodiment of the invention, the formulation comprises an antibody as angiogenesis inhibitor.

In one embodiment of the invention, the angiogenesis inhibitor is selected from the list of bevacizumab, ramucirumab, and ranibizumab.

In one embodiment of the invention, the angiogenesis inhibitor is an antibody selected from the list of bevacizumab, ramucirumab, and ranibizumab, particularly bevacizumab.

In one embodiment of the invention, the angiogenesis inhibitor is bevacizumab.

In one embodiment of the invention, the formulation comprises 1 wt % to 90 wt % of antibody or angiogenesis inhibitor, particularly 10 wt % to 80 wt % of antibody or angiogenesis inhibitor, more particularly 30 wt % to 60 wt % of antibody or angiogenesis inhibitor, most particularly 36 wt % to 44 wt % of antibody or angiogenesis inhibitor.

In one embodiment of the invention, the formulation comprises 1 wt % to 90 wt % of bevacizumab, particularly 10 wt % to 80 wt % of bevacizumab, more particularly 30 wt % to 60 wt % of bevacizumab, most particularly 36 wt % to 44 wt % of bevacizumab.

Two excipients are typically used in addition to the antibody or angiogenesis inhibitor, a stabilizer and a dispersant, the first to stabilize the API in the amorphous state, and a second to improve the dispersibility of the particles for aerosol delivery.

For monoclonal antibodies (mAb), irreversible aggregation of the individual mAb molecules must be prevented to preserve the biological activity and potency of the material. This aggregation typically occurs when hydrophobic domains of the antibody come into contact with each other in an otherwise hydrophilic environment, causing adhesion. To help prevent mAb aggregation, a hydrophilic stabilizer, such as trehalose (or another sugar), is included in the formulation. The trehalose molecules are believed to prevent exposure of hydrophobic domains, thereby reducing adhesion. To maintain the protective features of trehalose, it is critical that the trehalose molecules remain intimately mixed with the antibody molecules. To assess this, thermal analysis is performed on the spray dried material to confirm via DSC the phase of the mixture of trehalose and antibody.

Trehalose (CAS number 99-20-7) is a non-reducing sugar consisting of two molecules of glucose.

For protein and antibody actives, trehalose is strongly preferred as the stabilizing excipient. For small-molecule actives, other non-reducing sugars may be suitable, such as mannitol, raffinose, cyclodextrins, inulin and pullulan.

In one embodiment of the invention, the formulation further comprises a stabilizer.

In a particular embodiment of the invention, the formulation comprises a stabilizer selected from the list of trehalose, mannitol, raffinose, α-cyclodextrin, β-cyclodextrin, γ-cyclodextrin, inulin, pullulan and mixtures thereof, particularly trehalose.

In a particular embodiment of the invention, the formulation comprises trehalose as stabilizer.

In a particular embodiment of the invention, the formulation comprises 10 wt % to 90 wt % of stabilizer, particularly 20 wt % to 80 wt % of stabilizer, more particularly 30 wt % to 80 wt % of stabilizer, most particularly 36 wt % to 44 wt % of stabilizer.

In a particular embodiment of the invention, the formulation comprises 10 wt % to 90 wt % of trehalose, particularly 20 wt % to 80 wt % of trehalose, more particularly 30 wt % to 80 wt % of trehalose, most particularly 36 wt % to 44 wt % of trehalose.

Particle cohesion is a common issue which can prevent delivery to the deep lung due to agglomeration of particles into clusters. To reduce particle cohesion, a pharmaceutically acceptable excipient for dispersibility enhancement (dispersant) is added to the formulation. One particular dispersant is L-leucine, which has been shown in the literature to form a crystalline shell around the outside of the spray dried particle, which helps powders to flow better and reduce inter-particle attraction. To confirm that L-leucine is functioning properly in the formulation, PXRD is used to evaluate whether the L-leucine is crystalline in form, as opposed to amorphous angiogenesis inhibitor and stabilizer.

L-leucine (CAS number 61-90-5) is the L-enantiomer of leucine, an essential alpha amino acid used in the biosynthesis of proteins.

Tri-leucine (CAS number 10329-75-6) is a tripeptide formed from three L-leucine residues.

L-Isoleucine (CAS number 73-32-5) is the L-enantiomer of isoleucine, an essential alpha amino acid used in the biosynthesis of proteins.

The preferred excipients for dispersibility enhancement are L-leucine, L-isoleucine, and tri-leucine.

Additional suitable amino acid excipients include arginine, histidine, and glycine. Most preferred excipient for dispersibility enhancement (dispersant) is L-leucine.

In one embodiment of the invention, the formulation further comprises a dispersant.

In a particular embodiment of the invention, the formulation comprises one or more amino acids as dispersant.

In a particular embodiment of the invention, the formulation comprises a dispersant selected from L-leucine, tri-leucine, L-isoleucine, arginine, histidine, glycine, and mixtures thereof, particularly L-leucine.

In a particular embodiment of the invention, the formulation comprises a dispersant selected from L-leucine, tri-leucine, L-isoleucine, and mixtures thereof.

In a particular embodiment of the invention, the formulation comprises L-leucine as dispersant.

In a particular embodiment of the invention, the formulation comprises 2 wt % to 40 wt % of dispersant, particularly 5 wt % to 30 wt % of dispersant, more particularly 10 wt % to 25 wt % of dispersant, most particularly 18 wt % to 22 wt % of dispersant.

In a particular embodiment of the invention, the formulation comprises 2 wt % to 40 wt % of L-leucine, particularly 5 wt % to 30 wt % of L-leucine, more particularly 10 wt % to 25 wt % of L-leucine, most particularly 18 wt % to 22 wt % of L-leucine.

In one embodiment of the invention, the formulation further comprises a buffer.

In one embodiment of the invention, the formulation is essentially free of buffer.

In one embodiment of the invention, the formulation does not comprise a buffer.

In a particular embodiment of the invention, the formulation comprises a buffer selected from phosphate, tris(hydroxymethyl)aminomethane (TRIS), acetate, glycine, citric acid, carbonate, and mixtures thereof, particularly phosphate.

In a particular embodiment of the invention, the formulation comprises phosphate as buffer.

In a particular embodiment of the invention, the formulation comprises less than 5 wt % of buffer, particularly less than 1 wt % of buffer, more particularly less than 0.5 wt % of buffer.

In a particular embodiment of the invention, the formulation comprises 1 wt % to 2 wt % of buffer.

In a particular embodiment of the invention, the formulation comprises 1 wt % to 2 wt % of phosphate buffer.

In a particular embodiment of the invention, the formulation comprises about 1.7 wt % of phosphate buffer.

In a particular embodiment of the invention, the formulation comprises less than 1.7 wt % of phosphate buffer.

In a particular embodiment of the invention, the formulation comprises less than 1 wt % of phosphate buffer.

In one embodiment of the invention, the formulation comprises one or more antibodies or one or more angiogenesis inhibitors, one or more stabilizers, one or more dispersants and optionally one or more buffers.

In one embodiment of the invention, the formulation comprises 1 wt % to 90 wt % of antibody or angiogenesis inhibitor, 10 wt % to 90 wt % of stabilizer, 2 wt % to 40 wt % of dispersant, and optionally up to 5 wt % buffer, wherein the overall sum of concentrations of ingredients does not exceed 100 wt %.

In one embodiment of the invention, the formulation comprises 10 wt % to 80 wt % of antibody or angiogenesis inhibitor, 20 wt % to 80 wt % of stabilizer, 5 wt % to 30 wt % of dispersant, and optionally up to 5 wt % buffer, wherein the overall sum of concentrations of ingredients does not exceed 100 wt %.

In one embodiment of the invention, the formulation comprises 10 wt % to 80 wt % of antibody or angiogenesis inhibitor, 20 wt % to 80 wt % of stabilizer, 5 wt % to 30 wt % of dispersant, and less than 1 wt % buffer, wherein the overall sum of concentrations of ingredients does not exceed 100 wt %.

In one embodiment of the invention, the formulation comprises 10 wt % to 40 wt % of antibody or angiogenesis inhibitor, 40 wt % to 70 wt % of stabilizer, 10 wt % to 25 wt % of dispersant, and up to 5 wt % buffer, wherein the overall sum of concentrations of ingredients does not exceed 100 wt %.

In one embodiment of the invention, the formulation comprises 30 wt % to 60 wt % of antibody or angiogenesis inhibitor, 30 wt % to 80 wt % of stabilizer, 10 wt % to 25 wt % of dispersant, and less than 1 wt % buffer, wherein the overall sum of concentrations of ingredients does not exceed 100 wt %.

In one embodiment of the invention, the formulation comprises 30 wt % to 60 wt % of antibody or angiogenesis inhibitor, 30 wt % to 80 wt % of stabilizer, 10 wt % to 25 wt % of dispersant, and up to 5 wt % of buffer, wherein the overall sum of concentrations of ingredients does not exceed 100 wt %.

In one embodiment of the invention, the formulation comprises 30 wt % to 60 wt % of antibody or angiogenesis inhibitor, 30 wt % to 80 wt % of stabilizer, 10 wt % to 25 wt % of dispersant, and 1 wt % to 2 wt % of buffer, wherein the overall sum of concentrations of ingredients does not exceed 100 wt %.

In one embodiment of the invention, the formulation comprises 36 wt % to 44 wt % of antibody or angiogenesis inhibitor, 36 wt % to 44 wt % of stabilizer, 18 wt % to 22 wt % of dispersant, and up to 5 wt % of buffer, wherein the overall sum of concentrations of ingredients does not exceed 100 wt %.

In one embodiment of the invention, the formulation comprises 36 wt % to 44 wt % of antibody or angiogenesis inhibitor, 36 wt % to 44 wt % of stabilizer, 18 wt % to 22 wt % of dispersant, and 1 wt % to 2 wt % of buffer, wherein the overall sum of concentrations of ingredients does not exceed 100 wt %.

In one embodiment of the invention, the formulation comprises 36 wt % to 44 wt % of antibody or angiogenesis inhibitor, 36 wt % to 44 wt % of stabilizer, 18 wt % to 22 wt % of dispersant, and less than 1 wt % buffer, wherein the overall sum of concentrations of ingredients does not exceed 100 wt %.

In one embodiment of the invention, the formulation comprises 36 wt % to 44 wt % of antibody or angiogenesis inhibitor, 36 wt % to 44 wt % of stabilizer, 18 wt % to 22 wt % of dispersant, and no buffer, wherein the overall sum of concentrations of ingredients does not exceed 100 wt %.

In one embodiment of the invention, the formulation comprises about 40 wt % of antibody or angiogenesis inhibitor, about 40 wt % of stabilizer, about 20 wt % of dispersant and less than 1 wt % buffer, wherein the overall sum of concentrations of ingredients does not exceed 100 wt %.

In one embodiment of the invention, the formulation comprises about 40 wt % of antibody or angiogenesis inhibitor, about 40 wt % of stabilizer, about 20 wt % of dispersant and about 1 wt % to 2 wt % buffer, wherein the overall sum of concentrations of ingredients does not exceed 100 wt %.

In one embodiment of the invention, the formulation comprises 40 wt % of antibody or angiogenesis inhibitor, 40 wt % of stabilizer and 20 wt % of dispersant.

In one embodiment of the invention, the formulation comprises 1 wt % to 90 wt % of bevacizumab, 10 wt % to 90 wt % of trehalose, 2 wt % to 40 wt % of L-leucine, and optionally up to 5 wt % buffer, wherein the overall sum of concentrations of ingredients does not exceed 100 wt %.

In one embodiment of the invention, the formulation comprises 10 wt % to 80 wt % of bevacizumab, 20 wt % to 80 wt % of trehalose, 5 wt % to 30 wt % of L-leucine, and less than 1 wt % buffer, wherein the overall sum of concentrations of ingredients does not exceed 100 wt %.

In one embodiment of the invention, the formulation comprises 10 wt % to 80 wt % of bevacizumab, 20 wt % to 80 wt % of trehalose, 5 wt % to 30 wt % of L-leucine, and up to 5 wt % of buffer, wherein the overall sum of concentrations of ingredients does not exceed 100 wt %.

In one embodiment of the invention, the formulation comprises 10 wt % to 80 wt % of bevacizumab, 20 wt % to 80 wt % of trehalose, 5 wt % to 30 wt % of L-leucine, and 1 wt % to 2 wt % of buffer, wherein the overall sum of concentrations of ingredients does not exceed 100 wt %.

In one embodiment of the invention, the formulation comprises 10 wt % to 80 wt % of bevacizumab, 20 wt % to 80 wt % of trehalose, about 20 wt % of L-leucine, and 1 wt % to 2 wt % of buffer, wherein the overall sum of concentrations of ingredients does not exceed 100 wt %.

In one embodiment of the invention, the formulation comprises 30 wt % to 60 wt % of bevacizumab, 30 wt % to 80 wt % of trehalose, 10 wt % to 25 wt % of L-leucine, and less than 1 wt % buffer, wherein the overall sum of concentrations of ingredients does not exceed 100 wt %.

In one embodiment of the invention, the formulation comprises 30 wt % to 60 wt % of bevacizumab, 30 wt % to 80 wt % of trehalose, 10 wt % to 25 wt % of L-leucine, and up to 5 wt % of buffer, wherein the overall sum of concentrations of ingredients does not exceed 100 wt %.

In one embodiment of the invention, the formulation comprises 30 wt % to 60 wt % of bevacizumab, 30 wt % to 80 wt % of trehalose, about 20 wt % of L-leucine, and 1 wt % to 5 wt % ofbuffer, wherein the overall sum of concentrations of ingredients does not exceed 100 wt %.

In one embodiment of the invention, the formulation comprises 10 wt % to 40 wt % of bevacizumab, 40 wt % to 70 wt % of trehalose, 10 wt % to 25 wt % of L-leucine, and up to 5 wt % buffer, wherein the overall sum of concentrations of ingredients does not exceed 100 wt %.

In one embodiment of the invention, the formulation comprises 10 wt % to 40 wt % of bevacizumab, 40 wt % to 70 wt % of trehalose, about 20 wt % of L-leucine, and 1 wt % to 2 wt % buffer, wherein the overall sum of concentrations of ingredients does not exceed 100 wt %.

In a particular embodiment of the invention, the formulation comprises 9 wt % to 11 wt % of bevacizumab, 63 wt % to 77 wt % of trehalose, 18 wt % to 22 wt % of L-leucine, and less than 1 wt % phosphate, wherein the overall sum of concentrations of ingredients does not exceed 100 wt %.

In a particular embodiment of the invention, the formulation comprises 9 wt % to 11 wt % of bevacizumab, 63 wt % to 77 wt % of trehalose, 18 wt % to 22 wt % of L-leucine, and 1 wt % to 2 wt % of phosphate, wherein the overall sum of concentrations of ingredients does not exceed 100 wt %.

In a particular embodiment of the invention, the formulation comprises 18 wt % to 22 wt % of bevacizumab, 54 wt % to 66 wt % of trehalose, 18 wt % to 22 wt % of L-leucine, and less than 1 wt % phosphate, wherein the overall sum of concentrations of ingredients does not exceed 100 wt %.

In a particular embodiment of the invention, the formulation comprises 18 wt % to 22 wt % of bevacizumab, 54 wt % to 66 wt % of trehalose, 18 wt % to 22 wt % of L-leucine, and 1 wt % to 2 wt % of phosphate, wherein the overall sum of concentrations of ingredients does not exceed 100 wt %.

In a particular embodiment of the invention, the formulation comprises 36 wt % to 44 wt % of bevacizumab, 36 wt % to 44 wt % of trehalose, 18 wt % to 22 wt % of L-leucine, and no buffer, wherein the overall sum of concentrations of ingredients does not exceed 100 wt %.

In a particular embodiment of the invention, the formulation comprises 36 wt % to 44 wt % of bevacizumab, 36 wt % to 44 wt % of trehalose, 18 wt % to 22 wt % of L-leucine, and 1 wt % to 2 wt % of phosphate buffer, wherein the overall sum of concentrations of ingredients does not exceed 100 wt %.

In a particular embodiment of the invention, the formulation comprises 36 wt % to 44 wt % of bevacizumab, 36 wt % to 44 wt % of trehalose, 18 wt % to 22 wt % of L-leucine, and 1.5 wt % to 1.9 wt % of buffer, wherein the overall sum of concentrations of ingredients does not exceed 100 wt %.

In a particular embodiment of the invention, the formulation comprises 36 wt % to 44 wt % of bevacizumab, 36 wt % to 44 wt % of trehalose, 18 wt % to 22 wt % of L-leucine, and less than 1.7 wt % buffer, wherein the overall sum of concentrations of ingredients does not exceed 100 wt %.

In a particular embodiment of the invention, the formulation comprises 36 wt % to 44 wt % of bevacizumab, 36 wt % to 44 wt % of trehalose, 18 wt % to 22 wt % of L-leucine, and less than 1 wt % buffer, wherein the overall sum of concentrations of ingredients does not exceed 100 wt %.

In a particular embodiment of the invention, the formulation comprises 40 wt % of bevacizumab, 40 wt % of trehalose, and 20 wt % of L-leucine.

In a particular embodiment of the invention, the formulation comprises about 10 wt % to about 40 wt % of bevacizumab, about 40 wt % to about 70 wt % of trehalose, about 20 wt % of L-leucine, and less than 5 wt % buffer, wherein the overall sum of concentrations of ingredients does not exceed 100 wt %.

In a particular embodiment of the invention, the formulation comprises about 10 wt % to about 40 wt % of bevacizumab, about 40 wt % to about 70 wt % of trehalose, about 20 wt % of L-leucine, and about 1 wt % to about 2 wt % buffer, wherein the overall sum of concentrations of ingredients does not exceed 100 wt %.

In a particular embodiment of the invention, the formulation comprises 9 wt % to 44 wt % of bevacizumab, 36 wt % to 77 wt % of trehalose, 18 wt % to 22 wt % of L-leucine, and 0.9 wt % to 2.2 wt % buffer, wherein the overall sum of concentrations of ingredients does not exceed 100 wt %.

One embodiment of the invention relates to a dry powder formulation suitable for administration via inhalation comprising an antibody or angiogenesis inhibitor, a small molecular API, a stabilizer, and a dispersant.

One embodiment of the invention relates to a formulation as described herein further comprising a small molecular API, i.e. the formulation is a fixed-dose combination.

In a particular embodiment the small molecular API has an aqueous solubility of at least 0.5 mg/mL, particularly of at least 1 mg/mL.

In a particular embodiment the small molecular API is commonly used in lung cancer first-line treatment.

In a particular embodiment the small molecular API is selected from cisplatin (CAS Reg. No. 15663-27-1), carboplatin (CAS Reg. No. 41575-94-4), topotecan (CAS Reg. No. 123948-87-8), paclitaxel (CAS Reg. No. 33069-62-4), and erlotinib (CAS Reg. No. 183321-74-6).

One embodiment of the invention relates to a dry powder formulation suitable for administration via inhalation comprising bevacizumab, trehalose, L-leucine, and a small molecular API selected from the list of cisplatin, carboplatin, topotecan, paclitaxel, and erlotinib.

In a particular embodiment the small molecular API is selected from cisplatin, paclitaxel, and erlotinib.

In a particular embodiment the small molecular API is cisplatin or carboplatin.

In a particular embodiment the small molecular API is cisplatin.

In a particular embodiment the small molecular API is carboplatin.

In a particular embodiment the small molecular API is topotecan.

In a particular embodiment the small molecular API is paclitaxel.

In a particular embodiment the small molecular API is erlotinib.

In one embodiment of the invention, the formulation is a fixed-dose combination comprising one single type of dual-API SDDs, i.e. SDDs comprising a small molecular API and an angiogenesis inhibitor, particularly wherein the majority of SDD particles comprises both active ingredients (small molecular API and angiogenesis inhibitor), more particularly wherein each SDD particle comprises both active ingredients (small molecular API and angiogenesis inhibitor).

In a particular embodiment of the invention, the dual-API SDDs are prepared by spray drying one single spray solution comprising a small molecular API and an angiogenesis inhibitor.

In one embodiment of the invention, the formulation is a fixed-dose combination comprising two types of co-sprayed mono-API SDDs, wherein the first type of mono-API SDDs comprises a small molecular API and wherein the second type of mono-API SDDs comprises an angiogenesis inhibitor, i.e. no SDD particle comprises both active ingredients (small molecular API and angiogenesis inhibitor).

In a particular embodiment of the invention, the fixed-dose combination comprising two types of co-sprayed mono-API SDDs is prepared by co-spray drying two spray solutions, wherein the first spray solution comprises a small molecular API, and wherein the second spray solution comprises an angiogenesis inhibitor.

In one embodiment of the invention, the formulation is a fixed-dose combination comprising an angiogenesis inhibitor, a small molecular API, a stabilizer, and a dispersant.

In one embodiment of the invention, the formulation is a fixed-dose combination comprising an angiogenesis inhibitor, a small molecular API, a stabilizer, a dispersant, and optionally a buffer.

In one embodiment of the invention, the formulation is a fixed-dose combination comprising bevacizumab, a small molecular API, trehalose, and about 20 wt % of L-leucine.

In one embodiment of the invention, the formulation is a fixed-dose combination comprising bevacizumab, trehalose, L-leucine, and a small molecular API selected from the list of cisplatin, carboplatin, topotecan, paclitaxel, and erlotinib.

In one embodiment of the invention, the formulation is a fixed-dose combination comprising bevacizumab, 5 wt % to 70 wt % of trehalose, about 20 wt % of L-leucine, and a small molecular API selected from the list of cisplatin, carboplatin, topotecan, paclitaxel, and erlotinib.

In one embodiment of the invention, the formulation is a fixed-dose combination comprising bevacizumab, 5 wt % to 70 wt % of trehalose, about 20 wt % of L-leucine, up to 5 wt % of buffer, and a small molecular API selected from the list of cisplatin, carboplatin, topotecan, paclitaxel, and erlotinib.

In one embodiment of the invention, the formulation is a fixed-dose combination comprising bevacizumab, 5 wt % to 70 wt % of trehalose, about 20 wt % of L-leucine, less than 1 wt % of buffer, and a small molecular API selected from the list of cisplatin, carboplatin, topotecan, paclitaxel, and erlotinib.

In one embodiment of the invention, the formulation is a fixed-dose combination comprising 5 wt % to 70 wt % bevacizumab, 5 wt % to 70 wt % of trehalose, about 20 wt % of L-leucine, and 5 wt % to 70 wt % small molecular API selected from the list of cisplatin, carboplatin, topotecan, paclitaxel, and erlotinib, wherein the overall sum of concentrations of ingredients does not exceed 100 wt %.

In one embodiment of the invention, the formulation is a fixed-dose combination comprising 5 wt % to 70 wt % bevacizumab, 5 wt % to 70 wt % of trehalose, about 20 wt % of L-leucine, up to 5 wt % of phosphate buffer and 5 wt % to 70 wt % small molecular API selected from the list of cisplatin, carboplatin, topotecan, paclitaxel, and erlotinib, wherein the overall sum of concentrations of ingredients does not exceed 100 wt %.

In one embodiment of the invention, the formulation is a fixed-dose combination comprising bevacizumab, 20 wt % to 60 wt % of trehalose, about 20 wt % of L-leucine, and a small molecular API selected from the list of cisplatin, carboplatin, topotecan, paclitaxel, and erlotinib.

In one embodiment of the invention, the formulation is a fixed-dose combination comprising 10 wt % to 40 wt % bevacizumab, 20 wt % to 60 wt % of trehalose, about 20 wt % of L-leucine, and 5 wt % to 60 wt % small molecular API selected from the list of cisplatin, carboplatin, topotecan, paclitaxel, and erlotinib, wherein the overall sum of concentrations of ingredients does not exceed 100 wt %.

In one embodiment of the invention, the formulation is a fixed-dose combination comprising 10 wt % to 40 wt % bevacizumab, 20 wt % to 60 wt % of trehalose, about 20 wt % of L-leucine, up to 5 wt % of phosphate buffer and 5 wt % to 60 wt % small molecular API selected from the list of cisplatin, carboplatin, topotecan, paclitaxel, and erlotinib, wherein the overall sum of concentrations of ingredients does not exceed 100 wt %.

In one embodiment of the invention, the formulation is a fixed-dose combination comprising 10 wt % to 40 wt % of bevacizumab, 20 wt % to 60 wt % of trehalose, about 20 wt % of L-leucine, and 5 wt % to 40 wt % of a small molecular API selected from the list of cisplatin, carboplatin, topotecan, paclitaxel, and erlotinib, wherein the overall sum of concentrations of ingredients does not exceed 100 wt %.

One embodiment of the invention relates to a formulation as described herein further comprising 1 wt % to 80 wt % of a small molecular API.

One embodiment of the invention relates to a formulation as described herein further comprising 1 wt % to 80 wt % of a small molecular API selected from the list of cisplatin, carboplatin, topotecan, paclitaxel, and erlotinib.

One embodiment of the invention relates to a formulation as described herein further comprising 10 wt % to 80 wt % of a small molecular API.

One embodiment of the invention relates to a formulation as described herein further comprising 10 wt % to 80 wt % of a small molecular API selected from the list of cisplatin, carboplatin, topotecan, paclitaxel, and erlotinib.

One embodiment of the invention relates to a formulation as described herein further comprising 1 wt % to 50 wt % of a small molecular API.

One embodiment of the invention relates to a formulation as described herein further comprising 1 wt % to 50 wt % of a small molecular API selected from the list of cisplatin, carboplatin, topotecan, paclitaxel, and erlotinib.

One embodiment of the invention relates to a formulation as described herein further comprising 5 wt % to 40 wt % of a small molecular API.

One embodiment of the invention relates to a formulation as described herein further comprising 5 wt % to 40 wt % of a small molecular API selected from the list of cisplatin, carboplatin, topotecan, paclitaxel, and erlotinib.

In one embodiment of the invention, the formulation comprises 1 wt % to 50 wt % of angiogenesis inhibitor, 1 wt % to 50 wt % of small molecular API, 10 wt % to 88 wt % of stabilizer, 5 wt % to 30 wt % of dispersant, and optionally up to 5 wt % buffer, wherein the overall sum of concentrations of ingredients does not exceed 100 wt %.

In one embodiment of the invention, the formulation comprises 1 wt % to 50 wt % of angiogenesis inhibitor, 1 wt % to 50 wt % of small molecular API, 10 wt % to 88 wt % of stabilizer, 5 wt % to 30 wt % of dispersant, and less than 1 wt % buffer, wherein the overall sum of concentrations of ingredients does not exceed 100 wt %.

In one embodiment of the invention, the formulation comprises 1 wt % to 50 wt % of angiogenesis inhibitor, 1 wt % to 50 wt % of small molecular API, 10 wt % to 88 wt % of stabilizer, 5 wt % to 30 wt % of dispersant, and 1 wt % to 2 wt % of buffer, wherein the overall sum of concentrations of ingredients does not exceed 100 wt %.

In one embodiment of the invention, the formulation comprises 5 wt % to 40 wt % of angiogenesis inhibitor, 5 wt % to 40 wt % of small molecular API, 20 wt % to 80 wt % of stabilizer, 10 wt % to 25 wt % of dispersant, and less than 1 wt % buffer, wherein the overall sum of concentrations of ingredients does not exceed 100 wt %.

In one embodiment of the invention, the formulation comprises 5 wt % to 40 wt % of bevacizumab, 20 wt % to 80 wt % of trehalose, 10 wt % to 25 wt % of L-leucine, and 5 wt % to 40 wt % of a small molecular API selected from the list of cisplatin, carboplatin, topotecan, paclitaxel, and erlotinib, wherein the overall sum of concentrations of ingredients does not exceed 100 wt %.

In one embodiment of the invention, the formulation comprises 10 wt % to 40 wt % of bevacizumab, 20 wt % to 60 wt % of trehalose, about 20 wt % of L-leucine, and 5 wt % to 40 wt % of a small molecular API selected from the list of cisplatin, carboplatin, topotecan, paclitaxel, and erlotinib, wherein the overall sum of concentrations of ingredients does not exceed 100 wt %.

In one embodiment of the invention, the formulation comprises 10 wt % to 40 wt % of bevacizumab, 20 wt % to 60 wt % of trehalose, about 20 wt % of L-leucine, up to 5 wt % of phosphate buffer and 5 wt % to 40 wt % of a small molecular API selected from the list of cisplatin, carboplatin, topotecan, paclitaxel, and erlotinib, wherein the overall sum of concentrations of ingredients does not exceed 100 wt %.

In one embodiment of the invention, the formulation comprises 10 wt % to 40 wt % of bevacizumab, 20 wt % to 60 wt % of trehalose, about 20 wt % of L-leucine, 1 wt % to 2 wt % of phosphate buffer and 5 wt % to 40 wt % of a small molecular API selected from the list of cisplatin, carboplatin, topotecan, paclitaxel, and erlotinib, wherein the overall sum of concentrations of ingredients does not exceed 100 wt %.

In one embodiment of the invention, the formulation comprises 1 wt % to 50 wt % of bevacizumab, 1 wt % to 50 wt % of cisplatin, 10 wt % to 88 wt % of trehalose, 5 wt % to 30 wt % of L-leucine, and optionally up to 5 wt % buffer, wherein the overall sum of concentrations of ingredients does not exceed 100 wt %.

In one embodiment of the invention, the formulation comprises 1 wt % to 50 wt % of bevacizumab, 1 wt % to 50 wt % of cisplatin, 10 wt % to 88 wt % of trehalose, 5 wt % to 30 wt % of L-leucine, and less than 1 wt % buffer, wherein the overall sum of concentrations of ingredients does not exceed 100 wt %.

In one embodiment of the invention, the formulation comprises 1 wt % to 50 wt % of bevacizumab, 1 wt % to 50 wt % of cisplatin, 10 wt % to 88 wt % of trehalose, 5 wt % to 30 wt % of L-leucine, and 1 wt % to 2 wt % of phosphate buffer, wherein the overall sum of concentrations of ingredients does not exceed 100 wt %.

In one embodiment of the invention, the formulation comprises 5 wt % to 40 wt % of bevacizumab, 5 wt % to 40 wt % of cisplatin, 20 wt % to 80 wt % of trehalose, 10 wt % to 25 wt % of L-leucine, and less than 1 wt % buffer, wherein the overall sum of concentrations of ingredients does not exceed 100 wt %.

In one embodiment of the invention, the formulation comprises 5 wt % to 40 wt % of bevacizumab, 5 wt % to 40 wt % of cisplatin, 20 wt % to 80 wt % of trehalose, 10 wt % to 25 wt % of L-leucine, and 1 wt % to 2 wt % of phosphate buffer, wherein the overall sum of concentrations of ingredients does not exceed 100 wt %.

In one embodiment of the invention, the formulation comprises about 20 wt % of bevacizumab, about 5 wt % of cisplatin, about 55 wt % of trehalose, and about 20 wt % of L-leucine, particularly in the form of Dual-API SDDs.

In one embodiment of the invention, the formulation comprises about 20 wt % of bevacizumab, about 5 wt % of cisplatin, about 55 wt % of trehalose, about 20 wt % of L-leucine, and up to 5 wt % of phosphate buffer, particularly in the form of Dual-API SDDs, wherein the overall sum of concentrations of ingredients does not exceed 100 wt %.

In one embodiment of the invention, the formulation comprises about 20 wt % of bevacizumab, about 10 wt % of cisplatin, about 50 wt % of trehalose, and about 20 wt % of L-leucine, particularly in the form of Dual-API SDDs.

In one embodiment of the invention, the formulation comprises about 20 wt % of bevacizumab, about 10 wt % of cisplatin, about 50 wt % of trehalose, about 20 wt % of L-leucine, and up to 5 wt % of phosphate buffer, particularly in the form of Dual-API SDDs, wherein the overall sum of concentrations of ingredients does not exceed 100 wt %.

In one embodiment of the invention, the formulation comprises about 20 wt % of bevacizumab, about 5 wt % of cisplatin, about 55 wt % of trehalose, and about 20 wt % of L-leucine, particularly in the form of co-sprayed mono-API SDDs.

In one embodiment of the invention, the formulation comprises about 20 wt % of bevacizumab, about 5 wt % of cisplatin, about 55 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises about 13.3 wt % of bevacizumab, about 6.7 wt % of cisplatin, about 60 wt % of trehalose, and about 20 wt % of L-leucine, particularly in the form of co-sprayed mono-API SDDs.

In one embodiment of the invention, the formulation comprises about 13.3 wt % of bevacizumab, about 6.7 wt % of cisplatin, about 60 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises 1 wt % to 50 wt % of bevacizumab, 1 wt % to 60 wt % of erlotinib, 10 wt % to 88 wt % of trehalose, 5 wt % to 30 wt % of L-leucine, and optionally up to 5 wt % buffer, wherein the overall sum of concentrations of ingredients does not exceed 100 wt %.

In one embodiment of the invention, the formulation comprises 1 wt % to 50 wt % of bevacizumab, 1 wt % to 50 wt % of erlotinib, 10 wt % to 88 wt % of trehalose, 5 wt % to 30 wt % of L-leucine, and less than 1 wt % buffer, wherein the overall sum of concentrations of ingredients does not exceed 100 wt %.

In one embodiment of the invention, the formulation comprises 1 wt % to 50 wt % of bevacizumab, 1 wt % to 50 wt % of erlotinib, 10 wt % to 88 wt % of trehalose, 5 wt % to 30 wt % of L-leucine, and 1 wt % to 2 wt % of phosphate buffer, wherein the overall sum of concentrations of ingredients does not exceed 100 wt %.

In one embodiment of the invention, the formulation comprises 5 wt % to 40 wt % of bevacizumab, 5 wt % to 40 wt % of erlotinib, 20 wt % to 80 wt % of trehalose, 10 wt % to 25 wt % of L-leucine, and less than 1 wt % buffer, wherein the overall sum of concentrations of ingredients does not exceed 100 wt %.

In one embodiment of the invention, the formulation comprises 5 wt % to 40 wt % of bevacizumab, 5 wt % to 40 wt % of erlotinib, 20 wt % to 80 wt % of trehalose, 10 wt % to 25 wt % of L-leucine, and 1 wt % to 2 wt % of phosphate buffer, wherein the overall sum of concentrations of ingredients does not exceed 100 wt %.

In one embodiment of the invention, the formulation comprises about 26.67 wt % of bevacizumab, about 26.67 wt % of erlotinib, about 26.67 wt % of trehalose, and about 20 wt % of L-leucine, particularly in the form of co-sprayed mono-API SDDs.

In one embodiment of the invention, the formulation comprises about 26.67 wt % of bevacizumab, about 26.67 wt % of erlotinib, about 26.67 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises about 20 wt % of bevacizumab, about 40 wt % of erlotinib, about 20 wt % of trehalose, and about 20 wt % of L-leucine, particularly in the form of co-sprayed mono-API SDDs.

In one embodiment of the invention, the formulation comprises about 20 wt % of bevacizumab, about 40 wt % of erlotinib, about 20 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises 1 wt % to 50 wt % of bevacizumab, 1 wt % to 70 wt % of paclitaxel, 10 wt % to 88 wt % of trehalose, 5 wt % to 30 wt % of L-leucine, and optionally up to 5 wt % buffer, wherein the overall sum of concentrations of ingredients does not exceed 100 wt %.

In one embodiment of the invention, the formulation comprises 1 wt % to 50 wt % of bevacizumab, 1 wt % to 60 wt % of paclitaxel, 10 wt % to 88 wt % of trehalose, 5 wt % to 30 wt % of L-leucine, and optionally up to 5 wt % buffer, wherein the overall sum of concentrations of ingredients does not exceed 100 wt %.

In one embodiment of the invention, the formulation comprises 1 wt % to 50 wt % of bevacizumab, 1 wt % to 50 wt % of paclitaxel, 10 wt % to 88 wt % of trehalose, 5 wt % to 30 wt % of L-leucine, and less than 1 wt % buffer, wherein the overall sum of concentrations of ingredients does not exceed 100 wt %.

In one embodiment of the invention, the formulation comprises 1 wt % to 50 wt % of bevacizumab, 1 wt % to 50 wt % of paclitaxel, 10 wt % to 88 wt % of trehalose, 5 wt % to 30 wt % of L-leucine, and 1 wt % to 2 wt % of phosphate buffer, wherein the overall sum of concentrations of ingredients does not exceed 100 wt %.

In one embodiment of the invention, the formulation comprises 5 wt % to 40 wt % of bevacizumab, 5 wt % to 40 wt % of paclitaxel, 20 wt % to 80 wt % of trehalose, 10 wt % to 25 wt % of L-leucine, and less than 1 wt % buffer, wherein the overall sum of concentrations of ingredients does not exceed 100 wt %.

In one embodiment of the invention, the formulation comprises 5 wt % to 40 wt % of bevacizumab, 5 wt % to 40 wt % of paclitaxel, 20 wt % to 80 wt % of trehalose, 10 wt % to 25 wt % of L-leucine, and 1 wt % to 2 wt % of phosphate buffer, wherein the overall sum of concentrations of ingredients does not exceed 100 wt %.

In one embodiment of the invention, the formulation comprises about 33.3 wt % of bevacizumab, about 13.3 wt % of paclitaxel, about 33.3 wt % of trehalose, and about 20 wt % of L-leucine, particularly in the form of co-sprayed mono-API SDDs.

In one embodiment of the invention, the formulation comprises about 33.3 wt % of bevacizumab, about 13.3 wt % of paclitaxel, about 33.3 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises about 26.67 wt % of bevacizumab, about 26.67 wt % of paclitaxel, about 26.67 wt % of trehalose, and about 20 wt % of L-leucine.

In one embodiment of the invention, the formulation comprises about 26.67 wt % of bevacizumab, about 26.67 wt % of paclitaxel, about 26.67 wt % of trehalose, and about 20 wt % of L-leucine, particularly in the form of co-sprayed mono-API SDDs.

In one embodiment of the invention, the formulation comprises about 26.67 wt % of bevacizumab, about 26.67 wt % of paclitaxel, about 26.67 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises about 20 wt % of bevacizumab, about 40 wt % of paclitaxel, about 20 wt % of trehalose, and about 20 wt % of L-leucine, particularly in the form of co-sprayed mono-API SDDs.

In one embodiment of the invention, the formulation comprises about 20 wt % of bevacizumab, about 40 wt % of paclitaxel, about 20 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises about 13.3 wt % of bevacizumab, about 53.3 wt % of paclitaxel, about 13.3 wt % of trehalose, and about 20 wt % of L-leucine, particularly in the form of co-sprayed mono-API SDDs.

In one embodiment of the invention, the formulation comprises about 13.3 wt % of bevacizumab, about 53.3 wt % of paclitaxel, about 13.3 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation comprises about 6.67 wt % of bevacizumab, about 66.67 wt % of paclitaxel, about 6.67 wt % of trehalose, and about 20 wt % of L-leucine, particularly in the form of co-sprayed mono-API SDDs.

In one embodiment of the invention, the formulation comprises about 6.67 wt % of bevacizumab, about 66.67 wt % of paclitaxel, about 6.67 wt % of trehalose, about 20 wt % of L-leucine, and up to 5 wt % of phosphate buffer, particularly in the form of co-sprayed mono-API SDDs, wherein the overall sum of concentrations of ingredients does not exceed 100 wt %.

In one embodiment of the invention, the formulation is particulate.

In one embodiment of the invention, the formulation is a powder.

In one embodiment of the invention, the formulation is a dry powder.

In one embodiment of the invention, the formulation is a solid dispersion.

In one embodiment of the invention, the formulation is a spray dried solid dispersion (SDD).

In one embodiment of the invention, the formulation is a spray dried solid dispersion with a particle size distribution of d90<50 μm, particularly d90<20 μm, more particularly d90<10 μm, even more particularly d90<8 μm, most particularly d90<5 μm.

In one embodiment of the invention, the formulation is a spray dried solid dispersion with a particle size distribution of d50<5 μm, more particularly d50<3 μm, most particularly d50<2.5 μm.

In one embodiment of the invention, the formulation is a spray dried solid dispersion with a particle size distribution of d10>100 nm, more particularly d10>500 nm, most particularly d10>1 μm.

In one embodiment of the invention, the formulation has a particle size distribution of d90<10 μm, d50<3 μm, and d10>500 nm.

In one embodiment of the invention, the formulation is a spray dried solid dispersion with a particle size distribution of d90<10 μm, d50<3 μm, and d10>500 nm.

In one embodiment of the invention, the formulation is a spray dried solid dispersion with an unimodal particle size distribution.

In one embodiment of the invention, the formulation has at ambient conditions an average moisture content of 1-20 wt %, particularly 3-8 wt %.

In one embodiment of the invention, the antibody or angiogenesis inhibitor is amorphous.

In one embodiment of the invention, the antibody or angiogenesis inhibitor comprises less than 10 wt % crystalline content.

In one embodiment of the invention, the dispersant is crystalline.

In one embodiment of the invention, the dispersant comprises less than 50 wt % amorphous content, particularly less than 20 wt % amorphous content, more particularly less than 10 wt % amorphous content.

In one embodiment of the invention, the antibody or angiogenesis inhibitor is dispersed in the formulation.

In one embodiment of the invention, the antibody or angiogenesis inhibitor is homogeneously or substantially homogeneously dispersed in the formulation.

In one embodiment of the invention, the antibody or angiogenesis inhibitor is intimately mixed with the other ingredients in the formulation.

In one embodiment of the invention, the antibody or angiogenesis inhibitor and the stabilizer are both amorphous.

In a particular embodiment of the invention, the antibody or angiogenesis inhibitor and the stabilizer together form one single, amorphous phase having one glass transition temperature Tg as measured by DSC of at least 20° C., preferably at least 30° C., more preferably at least 40° C., above room temperature.

In a particular embodiment of the invention, the antibody or angiogenesis inhibitor and the stabilizer together form one single, amorphous phase having one glass transition temperature Tg as measured by DSC, while the crystalline dispersant has a separate melt temperature Tm.

In a particular embodiment of the invention, the antibody or angiogenesis inhibitor and the stabilizer together form one single, amorphous phase having one glass transition temperature Tg as measured by DSC of at least 20° C., preferably at least 30° C., more preferably at least 40° C., above room temperature, while the crystalline dispersant has a separate melt temperature Tm.

In one embodiment of the invention, the antibody or angiogenesis inhibitor and the stabilizer each comprises less than 10 wt % crystalline content.

In one embodiment of the invention, the antibody or angiogenesis inhibitor and the stabilizer are both amorphous whereas the dispersant is crystalline or partially crystalline.

In one embodiment of the invention, bevacizumab and trehalose are both amorphous and L-leucine is crystalline or partially crystalline.

In one embodiment of the invention, the formulation comprises core-shell particles, wherein the core comprises a dispersion of antibody or angiogenesis inhibitor and stabilizer and wherein the shell comprises dispersant.

In one embodiment of the invention, the formulation comprises core-shell particles, wherein the core comprises a dispersion of bevacizumab and trehalose and wherein the shell comprises L-leucine.

In one embodiment of the invention, the formulation comprises core-shell particles, wherein the core comprises a dispersion of antibody or angiogenesis inhibitor and stabilizer and wherein the shell comprises dispersant, as determined by X-ray photoelectron spectroscopy (XPS).

In one embodiment of the invention, the formulation is a spray dried solid dispersion wherein the dispersant, particularly L-leucine, is enriched at the SDD particle surface.

In one embodiment of the invention, the formulation is a spray dried solid dispersion which is coated by a dispersant, particularly by L-leucine.

The flow properties of the formulation as described herein can be further tailored or optimized by adding coarse carrier particles, e.g. to prevent overly strong adherence. Carrier particles can also be added as bulking agent to tailor the dose. Carrier particles are selected from lactose, trehalose or mannitol particles, particularly from α-Lactose monohydrate particles. Carrier particles are typically larger than 90 μm.

Preferably, carrier particles exhibit rough fissured surfaces.

One embodiment of the invention relates to a secondary formulation comprising a formulation as described herein and additionally carrier particles selected from lactose, trehalose or mannitol particles, particularly α-Lactose monohydrate particles.

In one embodiment, the carrier particles are larger than 90 μm, particularly larger than 90 μm but smaller than 500 μm. In one embodiment, the carrier particles have a particle size distribution of d50 from 90 μm to 150 μm or 210 μm to 355 μm.

In one embodiment, the secondary formulation comprises up to 95% wt of carrier particles.

The flow properties of the formulation as described herein can be further tailored or optimized by adding fine particles (fines), particularly fine lactose particles, more particularly fine α-Lactose monohydrate particles. Fines are typically smaller than 20 μm, particularly smaller than 10 μm.

One embodiment of the invention relates to a secondary formulation comprising a formulation as described herein and additionally fine particles, particularly fine lactose particles, more particularly fine α-Lactose monohydrate particles.

In one embodiment, the secondary formulation comprises up to 20% wt of fine particles.

In one embodiment, the fine particles are smaller than 20 μm, particularly smaller than 10 μm. In one embodiment, the fine particles have a particle size distribution of d50 from 3 μm to 15 μm The flow properties of the formulation as described herein can be further tailored or optimized by adding surface active agents, e.g. as lubricant, as flow adjuvant, as flowability enhancer, as stabilizer by preventing moisture penetration, as adhesion reducer, or as force control agent. The surface active agent can be a soap, such as a metal stearate, e.g. magnesium stearate or sodium stearate. The surface active agent can be present as particle and/or as discontinuous film that is partially coating inhalation particles and/or carrier particles. Surface active agent particles are typically smaller than 20 μm.

One embodiment of the invention relates to a secondary formulation comprising a formulation as described herein and additionally a surface active agent, particularly a metal stearate, more particularly magnesium stearate or sodium stearate.

In one embodiment, the secondary formulation comprises up to 1.5% wt, particularly 0.01% wt to 1.5% wt, more particularly 0.5% wt to 0.75% wt of surface active agent.

In one embodiment, the surface active agent is present as particles, particularly particles smaller than 20 μm. In one embodiment, the surface active agent particles have a particle size distribution of d50 from 5 μm to 12 μm.

In one embodiment, the surface active agent is present as film, particularly as a discontinuous film which is coating 5% to 60% of the carrier particles' surfaces.

One embodiment of the invention relates to a capsule comprising the formulation or secondary formulation as described herein.

One embodiment of the invention relates to a capsule comprising 1 mg to 100 mg, particularly 2 mg to 50 mg, more particularly 5 mg to 20 mg, of the formulation or secondary formulation as described herein.

Another embodiment of the invention relates to a kit comprising a dry powder inhaler and one or more capsules comprising the formulation or secondary formulation as described herein.

In one embodiment of the invention the capsule is made from gelatin, PEGylated gelatin or hydroxypropyl methylcellulose (HPMC).

Another embodiment of the invention relates to a blister pack or blister strip comprising the formulation or secondary formulation as described herein.

Another embodiment of the invention relates to a dry powder inhaler comprising a blister pack or blister strip comprising the formulation or secondary formulation as described herein.

A further embodiment relates to a dry powder inhaler comprising a reservoir with the formulation or secondary formulation as described herein.

Briefly, in spray drying, excipients and active are co-dissolved or suspended into a common solvent, such as water, buffer, methanol, ethanol, acetone, etc., or mixtures thereof. The liquid is pumped through an atomizing nozzle which breaks the liquid up into small droplets and sprays them into a drying chamber. In the drying chamber, heated drying gas rapidly removes the solvent from the droplets, resulting in a powder. The powder is typically cyclonically collected, and sometimes dried further in a secondary drying process. The particle size, morphology and density depend on the parameters of the spray drying process, including but not limited to: liquid flow rate, atomizer type, atomization pressure, spray solution concentration, inlet temperature, outlet temperature, drying gas flow rate.

When water or buffer is used as the spray drying solvent, the throughput of the process is limited by capacity of the drying gas to evaporate the solvent. Raising the liquid flow rate too high will cause insufficient drying of the particles, reducing yield and increasing particle adhesion.

The outlet temperature must be sufficiently low that the physical and chemical stability of the particles are not negatively impacted. The outlet temperature must be sufficiently low that the amorphous domains of the SDD are unable to recrystallize. For a monoclonal antibody or protein active, the outlet temperature must not be high enough to degrade the protein structure, which commonly occurs at temperatures above 70-80° C. Both inlet and outlet temperatures must conversely be sufficiently high that enough drying takes place to prevent sticking of wet product to dryer surfaces and obtain sufficient yield.

Interestingly, it has been observed that no degradation or unfolding of antibody is observed until above 150° C., indicating that the antibody is exceptionally stable in the formulations according to the invention.

The cyclonic collection device must be sized such that powders down to an aerodynamic diameter of 1 micron can be collected with good yield.

One embodiment of the invention relates to a spray drying process suitable to manufacture formulations as described herein.

In one embodiment of the invention, the spray drying process suitable to manufacture a formulation as described herein, wherein the process comprises the following steps:
a) preparing a spray drying solution by dissolution of antibody or angiogenesis inhibitor, stabilizer, dispersant and optionally further ingredients in a spray drying solvent;
b) directing, particularly pumping, a drying gas at a particular inlet temperature at a particular drying gas flow rate into a drying chamber;
c) directing, particularly pumping, the spray drying solution at a particular liquid flow rate through an atomizing nozzle into the drying chamber, said drying gas exiting the drying chamber at an outlet temperature.
d) collecting the obtained particles.

One embodiment of the invention relates to a spray drying process suitable to manufacture a formulation as described herein which is a fixed-dose combination comprising one single type of dual-API SDDs as described herein, wherein the process comprises the following steps:
a) preparing a spray drying solution by dissolution of angiogenesis inhibitor, small molecular API, stabilizer, dispersant and optionally further ingredients in a spray drying solvent;
b) directing, particularly pumping, a drying gas at a particular inlet temperature at a particular drying gas flow rate into a drying chamber;
c) directing, particularly pumping, the spray drying solution at a particular liquid flow rate through an atomizing nozzle into the drying chamber, said drying gas exiting the drying chamber at an outlet temperature.
d) collecting the obtained particles.

In one embodiment of the invention the formulation is a fixed-dose combination comprising one angiogenesis inhibitor and a small molecular API, wherein the formulation is obtained or obtainable via a spray drying process with only one spray drying solution.

One embodiment of the invention relates to a spray drying process suitable to manufacture a formulation as described herein which is a fixed-dose combination comprising two types of co-sprayed mono-API SDDs as described herein, wherein the process comprises the following steps:
a1) preparing a first spray drying solution by dissolution of small molecular API, optional stabilizer, dispersant and optionally further ingredients in a spray drying solvent;
a2) preparing a second spray drying solution by dissolution of angiogenesis inhibitor, stabilizer, dispersant and optionally further ingredients in a spray drying solvent;
b) directing, particularly pumping, a drying gas at a particular inlet temperature at a particular drying gas flow rate into a drying chamber;
c1) directing, particularly pumping, the two spray drying solutions simultaneously at particular liquid flow rates through two separate two-fluid atomizing nozzles into the drying chamber, said drying gas exiting the drying chamber at an outlet temperature.
d) collecting the obtained particles.

In one embodiment of the invention, the total solids concentration of the spray drying solution in step a), al), or a2) is 5 mg/ml to 20 mg/ml.

In one embodiment of the invention, the total solids concentration of the spray drying solution in step a), al), or a2) is 7 mg/ml to 12 mg/ml.

In one embodiment of the invention, the total solids concentration of the spray drying solution in step a), al), or a2) is 10 mg/ml.

In one embodiment of the invention, the concentration of antibody or angiogenesis inhibitor, optional small molecular API, stabilizer, dispersant and optionally further ingredients of the spray drying solution in step a) or a2) is 5 mg/ml to 20 mg/ml.

In one embodiment of the invention, the concentration of antibody or angiogenesis inhibitor, optional small molecular API, stabilizer, dispersant and optionally further ingredients of the spray drying solution in step a) or a2) is 7 mg/ml to 12 mg/ml.

In one embodiment of the invention, the concentration of antibody or angiogenesis inhibitor, optional small molecular API, stabilizer, dispersant and optionally further ingredients of the spray drying solution in step a) or a2) is 10 mg/ml.

In one embodiment of the invention, the drying gas in step b) comprises air or nitrogen.

In one embodiment of the invention, the drying gas in step b) is air or nitrogen.

In one embodiment of the invention the drying gas inlet temperature in step b) is from 80° C. to 200° C., particularly from 90° C. to 170° C., more particularly from 100° C. to 150° C., most particularly 110° C. to 130° C., most particularly 120° C.

In one embodiment of the invention the ratio of the drying gas flow rate (in step b) to the liquid flow rate in step c) is from 10 to 250, particularly from 20 to 125, more particularly from 20 to 100, most particularly 75.

The ratio of drying gas flow rate to liquid flow rate is independent from the scale of the spray dryer employed. Spray dryer of any size can be employed to practice the spray drying process as described herein.

In one embodiment of the invention a lab scale spray dryer is employed to practice the spray drying process as described herein, wherein the drying gas flow rate in step c) is from 300 g/min to 600 g/min, particularly from 450 g/min to 500 g/min and wherein the liquid flow rate in step c) is from 1 g/min to 40 g/min, particularly from 2 g/min to 20 g/min, more particularly from 3 g/min to 10 g/min.

In one embodiment of the invention a large scale spray dryer is employed to practice the spray drying process as described herein, wherein the drying gas flow rate in step c) is from 1200 g/min to 4000 g/min, particularly from 1400 g/min to 3500 g/min and wherein the liquid flow rate in step c) is from 10 g/min to 300 g/min, particularly from 50 g/min to 200 g/min, more particularly from 75 g/min to 150 g/min.

In one embodiment of the invention, the atomizing nozzle in step c) or in step c1) is a two-fluid nozzle.

In one embodiment of the invention the atomizing nozzle in step c) or in step c1) is operated at an atomization pressure from 0.5 bar to 10 bar, particularly from 1 bar to 5 bar, more particularly from 1.5 bar to 4 bar, most particularly 1.7 bar.

In one embodiment of the invention the outlet temperature in step c) or in step c1) is from 35° C. to 80° C., particularly from 40° C. to 70° C., more particularly from 45° C. to 65° C., more particularly from 45° C. to 55° C., even more particularly 50° C. to 55° C., most particularly 50° C.

In one embodiment of the invention, the particles are collected in step d) using a cyclonic collection device.

In one embodiment of the invention, the spray drying solvent is water or an aqueous buffer solution.

In one embodiment of the invention, the spray drying solvent is a mixture of an aqueous solvent and an organic solvent.

In one embodiment of the invention, the aqueous solvent is water or an aqueous buffer solution.

In one embodiment of the invention, the aqueous solvent is an aqueous buffer solution.

In one embodiment of the invention the aqueous buffer solution is a phosphate buffer solution (PBS).

In one embodiment of the invention the aqueous buffer solution has a pH of 5 to 8, particularly a pH of 6 to 7, more particularly a pH of 6.2 to 6.4.

In one embodiment of the invention the spray drying solvent is an aqueous buffer solution having a pH of 5 to 8, particularly a pH of 6 to 7, more particularly a pH of 6.2 to 6.4.

In one embodiment of the invention the aqueous buffer solution has a concentration of 0.1 mM to 10 mM, particularly a concentration of 0.5 mM to 5 mM, more particularly a concentration of 0.9 mM to 1.1 mM.

In a particular embodiment the aqueous buffer solution is a phosphate buffer solution (PBS) having a pH of 6.2 to 6.4 and a concentration of 0.9 mM to 1.1 mM.

In one embodiment of the invention, the organic solvent is selected from methanol, ethanol, acetone, and mixtures thereof.

In one embodiment of the invention, the organic solvent is ethanol or methanol, particularly ethanol.

In one embodiment of the invention, the spray drying solvent is a mixture of an aqueous solvent and up to 90 wt % of an organic solvent.

In one embodiment of the invention, the spray drying solvent is a mixture of an aqueous solvent and up to 50 wt % of an organic solvent.

In one embodiment of the invention, the spray drying solvent is a mixture of an aqueous solvent and up to 20 wt % of an organic solvent.

In one embodiment of the invention, the spray drying solvent is a mixture of phosphate buffer solution and comprising up to 90 wt % ethanol.

In one embodiment of the invention, the spray drying solvent is a mixture of phosphate buffer solution and comprising up to 50 wt % ethanol.

In one embodiment of the invention, the spray drying solvent is a mixture of phosphate buffer solution and comprising up to 20 wt % ethanol.

The atomizing nozzle in step c) must be selected such that the resulting aerodynamic particle size (measured by Next Generation Impactor) of the particles falls in a range suitable for delivery to the lung.

In one embodiment of the invention, the atomizing nozzle in step c) yields particles suitable for delivery to the lung, wherein at least 30 wt % of the particles have an aerodynamic particle size (measured by Next Generation Impactor) between 500 nm and 5 μm, particularly wherein at least 50 wt % of the particles have an aerodynamic particle size (measured by Next Generation Impactor) between 500 nm and 5 μm, more particularly at least 70 wt % of the particles have an aerodynamic particle size (measured by Next Generation Impactor) between 500 nm and 5 μm.

In one embodiment of the invention, the atomizing nozzle in step c) yields particles suitable for delivery to the lung, wherein at least 30 wt % of the delivered dose (measured by Next Generation Impactor) has an aerodynamic particle size falling between 500 nm and 5 μm, particularly at least 50 wt % of the delivered dose (measured by Next Generation Impactor) has an aerodynamic particle size falling between 500 nm and 5 μm, more particularly at least 70 wt % of the delivered dose (measured by Next Generation Impactor) has an aerodynamic particle size falling between 500 nm and 5 μm To reduce growth of blood vessels in cancerous tumors, compounds which inhibit the Vascular Endothelial Growth Factor are often administered in combination with other therapeutics. However, anti-VEGF compounds reduce vascularization throughout healthy tissue in addition to the tumor. Intravenous administration of high doses of VEGF inhibitors can result in a side effect of fatal bleeding. As a result of this, patients with enhanced risk of bleeding are excluded from otherwise-beneficial anti-VEGF therapies.

If instead, anti-VEGF compounds were delivered locally to the tumor, systemic absorption could be limited. For lung cancer, administration of the anti-VEGF compound to the lung by an inhaled dosage form may help reduce dangerous side effects, allow reduced dose, and improve patient outcomes.

The active can be self-administered by the patient at home. The drug is delivered to the lung by a dry powder inhaler, which uses a blister pack, blister strip, reservoir or capsule to deliver a unit dose.

According to the approved label of Avastin® solution for intravenous infusion, bevacizumab is administered by IV at 5 mg/kg every 2 weeks, 7.5 or 15 mg/kg every 3 weeks. Anticipated is up to a 10× reduction in dose when delivered locally. The state of the art for lung cancer treatment using monoclonal antibodies involves two phases: primary and maintenance treatment. In primary treatment, a chemotherapeutic agent is commonly administered alongside the mAb via intravenous infusion, with treatment taking place at a hospital or infusion center. Continued therapy after conclusion of chemotherapy is called maintenance therapy, and may continue indefinitely until disease progression or patient death, typically administered every 3-4 weeks. Maintenance therapy is also conducted by intravenous infusion, and must therefore be performed in a clinical setting. This leads to high costs and poor patient compliance. A means of self-administering maintenance therapy would improve patient compliance, reduce costs, and enable more frequent administration to the patient, if desired.

Monoclonal antibodies for lung cancer treatment are typically administered at a high dose (˜15 mg/kg), leading to >1 gram doses for IV infusion. In the current state of the art, most mAbs cannot be formulated in a concentrated solution such that administration of 1 gram would be possible in a subcutaneous injection (which is typically limited to 2-5 mL in volume and 60 mg/mL in concentration). Thus, subcutaneous is not a feasible means to self-administer maintenance therapy. Oral therapy of monoclonal antibodies is also not feasible for systemic delivery of large molecules due to slow absorption and degradation in the GI tract. Thus, inhalation therapy is a superior means to deliver mAb to the affected organ for lung cancer.

One embodiment of the invention relates to formulations as described herein for use as therapeutically active substance.

One embodiment of the invention relates to formulations as described herein for use in the treatment, prevention, delay of progression, and/or maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma.

One embodiment of the invention relates to the use of formulations as described herein for the treatment, prevention, delay of progression, and/or maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma.

One embodiment of the invention relates to the use of formulations as described herein for the preparation of a medicament useful for the treatment, prevention, delay of progression, and/or maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma.

One embodiment of the invention relates to a method of treatment, prevention, delay of progression, and/or maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma, which method comprises administering a formulation as described herein to a human being or animal.

One embodiment of the invention relates to formulations as described herein for use in maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma.

One embodiment of the invention relates to the use of formulations as described herein for maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma.

One embodiment of the invention relates to the use of formulations as described herein for the preparation of medicaments useful for maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma.

One embodiment of the invention relates to a method of maintenance therapy of asthma, COPD, lung infections, cystic fibrosis or lung cancer, particularly of lung cancer, particularly of non-small-cell lung carcinoma (NSCLC) and small cell lung carcinoma (SCLC), more particularly of lung adenocarcinoma, squamous cell carcinoma, epidermoid carcinoma, and large cell carcinoma, which method comprises administering a formulation as described herein to a human being or animal.

One embodiment of the invention relates to the sequential or concomitant administration of a formulation as described herein with a platinum-based chemotherapy such as carboplatin and cisplatin or with topotecan or erlotinib.

One embodiment of the invention relates to the sequential or concomitant use of a formulation as described herein and a dosage form comprising carboplatin, cisplatin, topotecan, or erlotinib.

One embodiment of the invention relates to the sequential or concomitant use of a formulation as described herein and a dosage form comprising carboplatin, cisplatin, topotecan, or erlotinib for the treatment or prevention of the treatment, prevention and/or delay of progression of lung cancer, particularly of non-small-cell lung carcinoma (NSCLC) and small cell lung carcinoma (SCLC), more particularly lung adenocarcinoma, squamous cell carcinoma, epidermoid carcinoma, and large cell carcinoma.

One embodiment of the invention relates to a method of the treatment, prevention and/or delay of progression of lung cancer, particularly of non-small-cell lung carcinoma (NSCLC) and small cell lung carcinoma (SCLC), more particularly lung adenocarcinoma, squamous cell carcinoma, epidermoid carcinoma, and large cell carcinoma, which method comprises sequential or concomitant administration of formulation as described herein and a dosage form comprising carboplatin, cisplatin, topotecan, or erlotinib to a human being or animal.

In one embodiment of the invention the formulation as described herein is administered via inhalation twice daily, once daily, twice weekly, once weekly, every two weeks or every three weeks.

In one embodiment of the invention the formulation as described herein is administered via inhalation at a daily overall antibody or angiogenesis inhibitor dose, particularly a daily overall bevacizumab dose, of 0.1 mg to 50 mg, particularly 0.1 mg to 20 mg, more particularly 1 mg to 10 mg.

In one embodiment of the invention the fixed-dose combination as described herein is administered via inhalation at a daily overall cisplatin dose of 0.01 mg to 20 mg, particularly 0.1 mg to 20 mg, more particularly 0.5 mg to 10 mg.

In one embodiment of the invention the fixed-dose combination as described herein is administered via inhalation at a daily overall carboplatin dose of 0.05 mg to 100 mg, particularly 1 mg to 50 mg, more particularly 1 mg to 20 mg.

In one embodiment of the invention the fixed-dose combination as described herein is administered via inhalation at a daily overall topotecan dose of 0.001 mg to 5 mg, particularly 0.01 mg to 1 mg, more particularly 0.05 mg to 0.6 mg.

In one embodiment of the invention the fixed-dose combination as described herein is administered via inhalation at a daily overall paclitaxel dose of 0.01 mg to 50 mg, particularly 0.1 mg to 20 mg, more particularly 0.5 mg to 10 mg.

In one embodiment of the invention the fixed-dose combination as described herein is administered via inhalation at a daily overall erlotinib dose of 1 mg to 150 mg, particularly 1 mg to 50 mg, more particularly 5 mg to 30 mg.

In one embodiment of the invention the formulation as described herein is administered via inhalation daily at a daily overall antibody or angiogenesis inhibitor dose, particularly a daily overall bevacizumab dose, of 0.1 mg to 50 mg, particularly 0.1 mg to 20 mg, more particularly 1 mg to 10 mg.

In one embodiment of the invention the formulation as described herein is administered via inhalation every two weeks at a bi-weekly overall antibody or angiogenesis inhibitor dose, particularly a bi-weekly overall bevacizumab dose, of 1 mg to 200 mg, particularly 1 mg to 150 mg, more particularly 10 mg to 100 mg.

### EXAMPLES

The following examples 1-26 are provided for illustration of the invention. They should not be considered as limiting the scope of the invention, but merely as being representative thereof.

Examples 1 to 5 provide exemplifications of SDDs comprising an angiogenesis inhibitor (Bevacizumab).

Examples 6 to 7 provide exemplifications of SDDs from one spray solution comprising an angiogenesis inhibitor (Bevacizumab) and a small molecular API (Cisplatin).

Examples 8 to 16 provide exemplifications of SDDs from two co-sprayed solutions, one spray solution comprising an angiogenesis inhibitor (Bevacizumab) and the other spray solution comprising a small molecular API (Erlotinib, Paclitaxel or Cisplatin).

Examples 17 to 26 provide additional or comparative examples.

Compositions of SDDs of Examples 1 to 16 are summarized in Table 2.

TABLE 2
Compositions of SDDs of Examples 1 to 16.

| | | co-spray ratio | | | L- | | | | |
|---|---|---|---|---|---|---|---|---|---|
| | | s.m. API SDD: | Bevacizumab | Trehalose | Leucine | Cisplatin | Erlotinib | Paclitaxel | Total |
| Ex. | SDD type | Bevac. SDD | [wt %] | [wt %] | [wt %] | [wt %] | [wt %] | [wt %] | [wt %] |
| 1 | mono-API | n.a. | 10 | 70 | 20 | — | — | — | 100 |
| 2 | mono-API | n.a. | 10 | 70 | 20 | — | — | — | 100 |
| 3 | mono-API | n.a. | 20 | 60 | 20 | — | — | — | 100 |
| 4 | mono-API | n.a. | 40 | 40 | 20 | — | — | — | 100 |
| 6 | dual-API | n.a. | 20 | 55 | 20 | 5 | — | — | 100 |
| 7 | dual-API | n.a. | 20 | 50 | 20 | 10 | — | — | 100 |
| 8 | co-sprayed | 1:2 | 26.67 | 26.67 | 20 | — | 26.67 | — | 100 |
| 9 | co-sprayed | 1:1 | 20 | 20 | 20 | — | 40 | — | 100 |
| 10 | co-sprayed | 1:5 | 33.33 | 33.33 | 20 | — | — | 13.33 | 100 |
| 11 | co-sprayed | 1:2 | 26.67 | 26.67 | 20 | — | — | 26.67 | 100 |
| 12 | co-sprayed | 1:1 | 20 | 20 | 20 | — | — | 40 | 100 |
| 13 | co-sprayed | 2:1 | 13.33 | 13.33 | 20 | — | — | 53.33 | 100 |
| 14 | co-sprayed | 5:1 | 6.67 | 6.67 | 20 | — | — | 66.67 | 100 |
| 15 | co-sprayed | 2:1 | 13.33 | 60 | 20 | 6.67 | — | — | 100 |
| 16 | co-sprayed | 1:1 | 20 | 55 | 20 | 5 | — | — | 100 |

### Example 1. Preparation of SDDs Comprising 10 wt % Bevacizumab/70 wt % Trehalose/20 wt % L-Leucine and In Vitro Studies Thereof

Bevacizumab was supplied as a sterile solution of 30 mg/mL bevacizumab in 50-mM phosphate buffer solution (PBS), pH 6.2, with 60 mg/mL trehalose and 0.04% Polysorbate 20. Bevacizumab solution as received was placed inside a SnakeSkin™ dialysis membrane clipped on both ends (10,000-Dalton molecular-weight cutoff, Fisher Scientific). This was floated in 1-mM sodium phosphate buffer with 20 mg/mL trehalose, at a volume ratio of 1:100, and gently stirred for 24 hours with one buffer exchange.

A spray solution was prepared containing 1 mg/mL bevacizumab, 7 mg/mL trehalose and 2 mg/mL L-leucine in pH 6.3 1 mM phosphate buffer. The spray solutions were spray dried in a pre-heated custom laboratory-scale spray dryer at a solution feed rate of 6 g/min, an inlet temperature of 120° C., outlet temperature of 50° C. and a drying gas flow rate of 500 g/min. The solution was atomized through a two-fluid nozzle (Model % J, with a 1650 liquid body and 64 air cap; Spraying Systems Co) at a pressure of 25 psi. The spray dried particles were collected by a 2″ cyclonic separator, dried under vacuum at ambient temperature with nitrogen sweep gas, and stored with desiccant at 5° C.

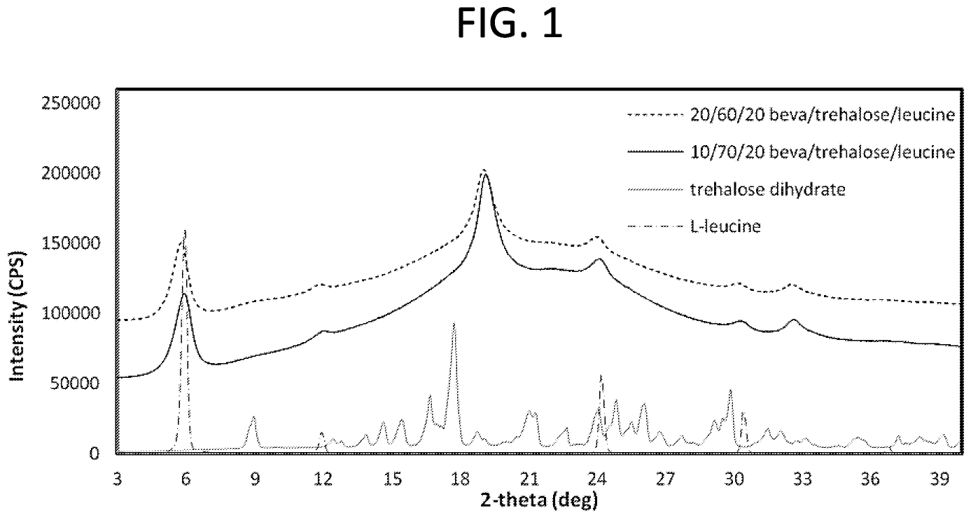

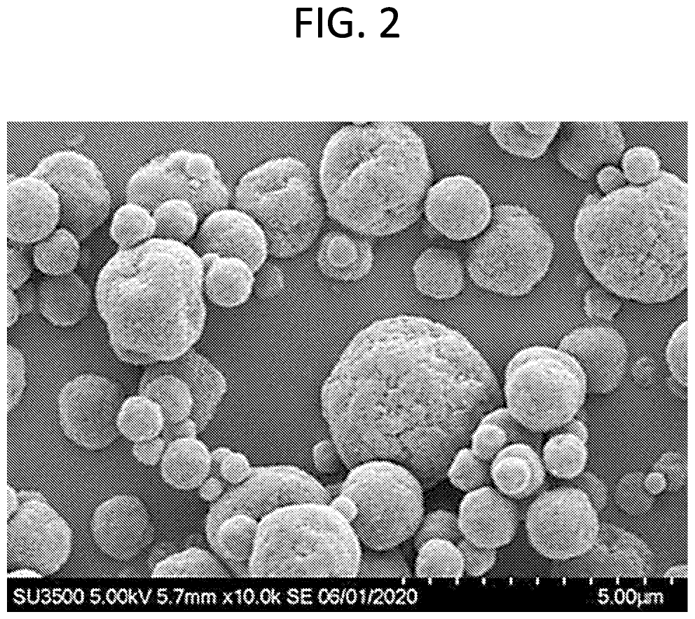

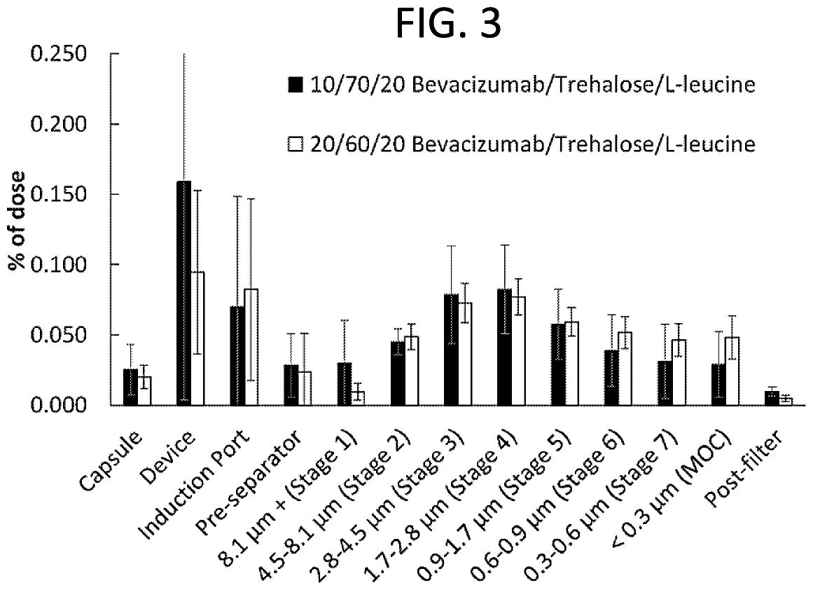

The L-leucine was crystalline by PXRD (FIG. 1), and the trehalose/mAb phase was amorphous by DSC. The morphology via SEM is represented in FIG. 2. The aerodynamic particle size distribution was measured using a Next Generation Impactor. The mass median aerodynamic diameter was 2.4 μm, the fine particle fraction was 66 wt % of the emitted dose, and the very fine particle fraction was 36 wt % of the emitted dose (FIG. 3).

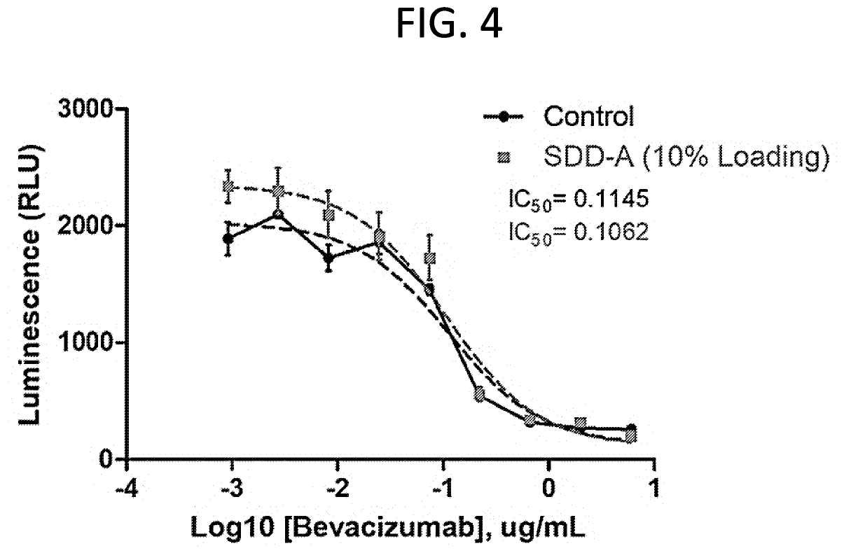

The SDD's biological activity to inhibit VEGF was measured using a VEGF bioassay (Promega, Inc product GA2001) compared with a bevacizumab control. The VEGF bioassay is a bioluminescent cell-based assay that measures VEGF stimulation and inhibition of KDR (VEGFR2) using the NFAT-RE as a readout. The KDR/NFAT-RE HEK293 cells have been engineered to express the NFAT response element upstream of Luc2P as well as exogenous KDR. When VEGF binds to the KDR/NFAT-RE HEK293 cells, the KDR transduces intracellular signals resulting in NFAT-RE-mediated luminescence. The bioluminescent signal is detected and quantified using Bio-Glo Luciferase Assay System and a standard luminometer. The IC50 for VEGF inhibition was 0.106 μg/mL for the SDD compared with 0.115 μg/mL for the control (FIG. 4).

After a 2-week stability challenge at 40° C./75% RH (vial closed with desiccant), the SDD retained similar aerodynamic properties and biologic activity. After the stability challenge, the MMAD was 2.1 μm, FPF was 72 wt % and vFPF was 40 wt %. The anti-VEGF IC50 was 0.095 μg/mL for the SDD and 0.092 μg/mL for the control. The L-leucine remained crystalline and the trehalose/mAb phase remained amorphous.

### Example 2. Preparation of SDDs Comprising 10 wt % Bevacizumab/70 wt % Trehalose/20 wt % L-Leucine and In Vitro Studies Thereof (Scale Up)

A spray solution was prepared containing 1 mg/mL bevacizumab, 7 mg/mL trehalose and 2 mg/mL L-leucine in pH 6.3 1 mM phosphate buffer. The spray solutions were spray dried in a pre-heated spray dryer at a solution feed rate of 40 g/min, an inlet temperature of 97° C., outlet temperature of 50° C. and a drying gas flow rate of 2800 g/min. The solution was atomized through a two-fluid nozzle (1650/120) at a pressure of 25 psi. The spray dried particles were collected by two 4″ cyclonic separator connected in parallel.

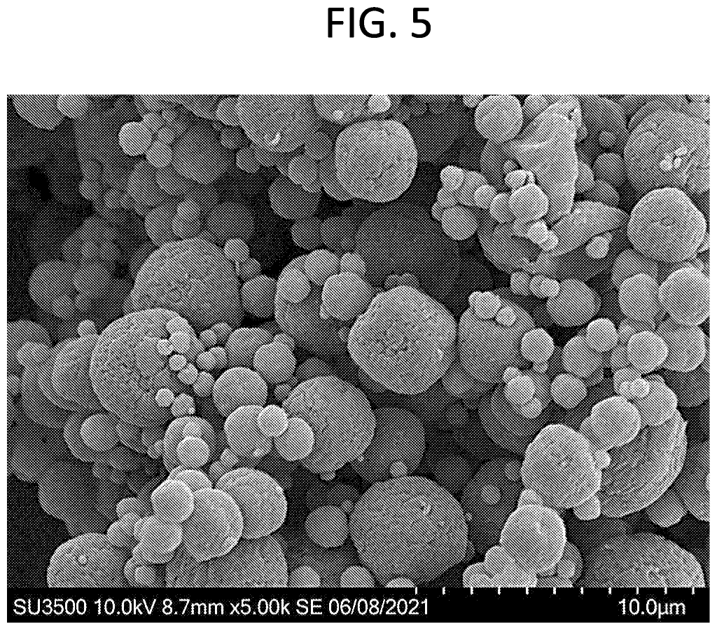

The L-leucine was crystalline by PXRD, and the trehalose/mAb phase was amorphous by DSC. The aerodynamic particle size distribution was measured using a Next Generation Impactor. The mass median aerodynamic diameter was 2.9 μm, the fine particle fraction was 69 wt % of the emitted dose. The geometric particle size distribution by laser light scattering revealed a d50 value of 2.5 μm and a d90 value of 5.5 μm. The morphology via SEM is represented in FIG. 5. The potency of the SDDs, measured by absorbance at 280 nm, was 10.1% bevacizumab, and the water content measured by Karl Fisher was 3.2% by weight. The SDD's biological activity to inhibit VEGF was measured using a Promega kit (See Example 1 for details) compared with a bevacizumab control. The IC50 for VEGF inhibition was 0.168 μg/mL for the SDD compared with 0.116 μg/mL for the control.

### Example 3. Preparation of SDDs Comprising 20 wt % Bevacizumab/60 wt % Trehalose/20 wt % L-Leucine and In Vitro Studies Thereof

A spray solution was prepared containing 2 mg/mL bevacizumab, 6 mg/mL trehalose and 2 mg/mL L-leucine in pH 6.3 1 mM phosphate buffer. The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator.

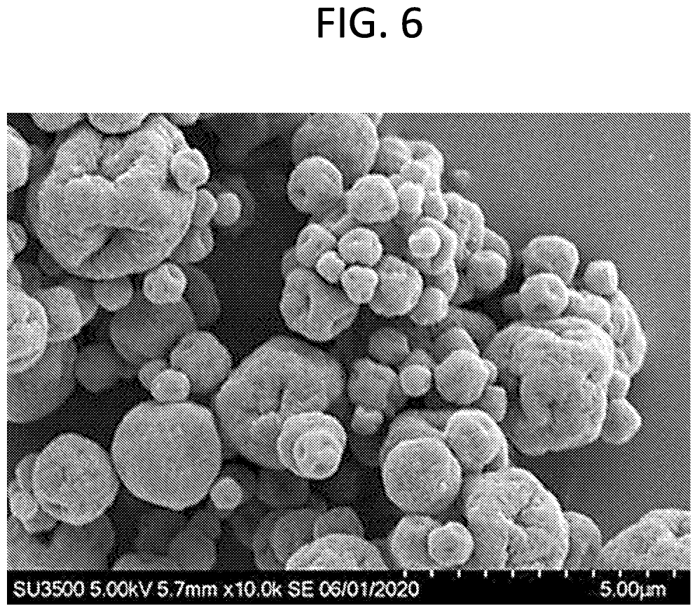

The L-leucine was crystalline by PXRD (FIG. 1), and the trehalose/mAb phase was amorphous by DSC. The aerodynamic particle size distribution was measured using a Next Generation Impactor. The mass median aerodynamic diameter was 1.6 μm, the fine particle fraction was 73 wt % of the emitted dose, and the very fine particle fraction was 36 wt % of the emitted dose (FIG. 3). The morphology via SEM is represented in FIG. 6.

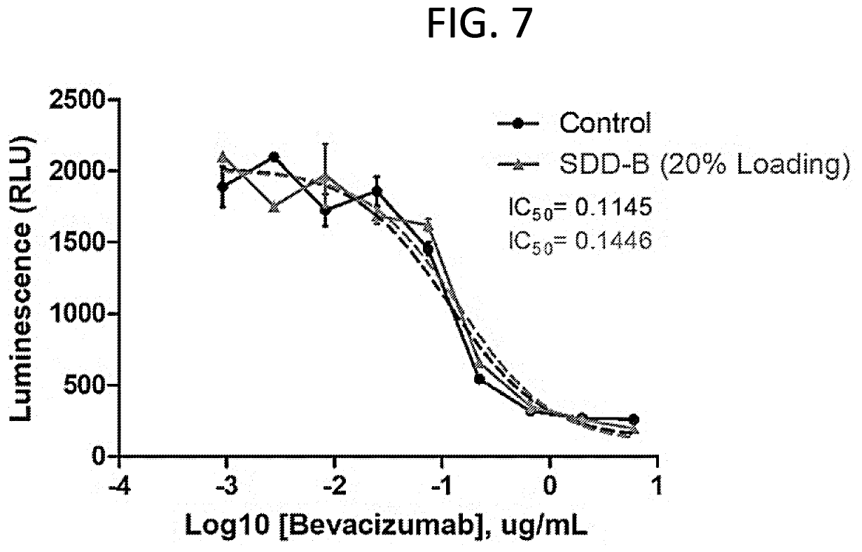

The SDD's biological activity to inhibit VEGF was measured using a Promega kit (see Example 1 for details) compared with a bevacizumab control. The IC50 for VEGF inhibition was 0.145 μg/mL for the SDD compared with 0.115 μg/mL for the control (FIG. 7).

After a 2-week stability challenge at 40° C./75% RH (vial closed with desiccant), the SDD retained similar aerodynamic properties and biologic activity. After the stability challenge, the MMAD was 2.1 μm, FPF was 74 wt % and vFPF was 41 wt %. The anti-VEGF IC50 was 0.127 μg/mL for the SDD and 0.092 μg/mL for the control. The L-leucine remained crystalline and the trehalose/mAb phase remained amorphous.

### Example 4. Preparation of SDDs Comprising 40 wt % Bevacizumab/40 wt % Trehalose/20 wt % L-Leucine and In Vitro Studies Thereof

A spray solution was prepared containing 4 mg/mL bevacizumab, 4 mg/mL trehalose and 2 mg/mL L-leucine in pH 6.3 1 mM phosphate buffer. The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator.

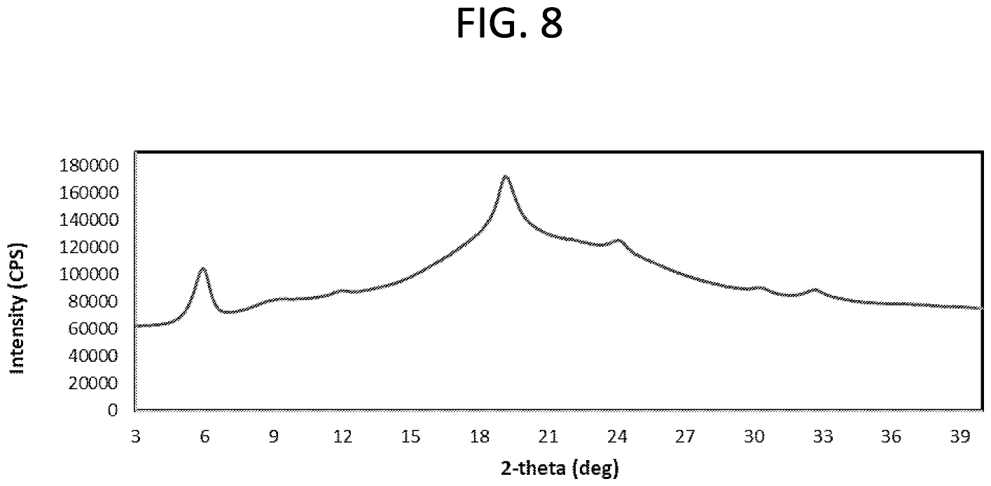

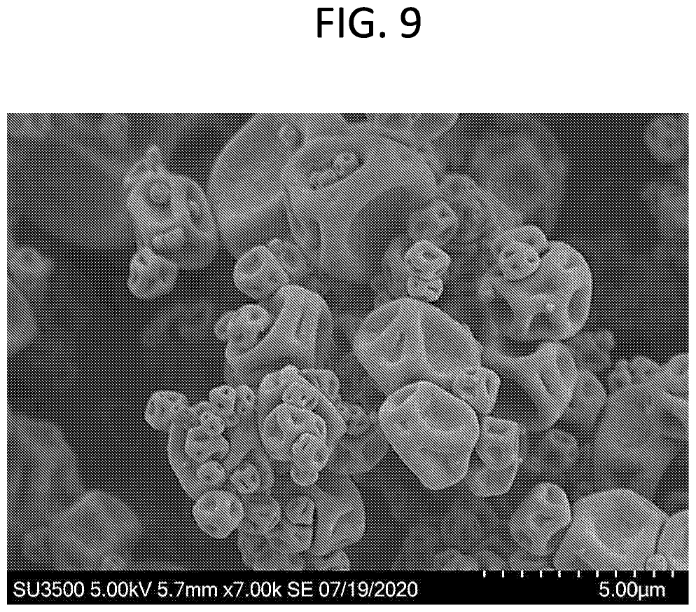

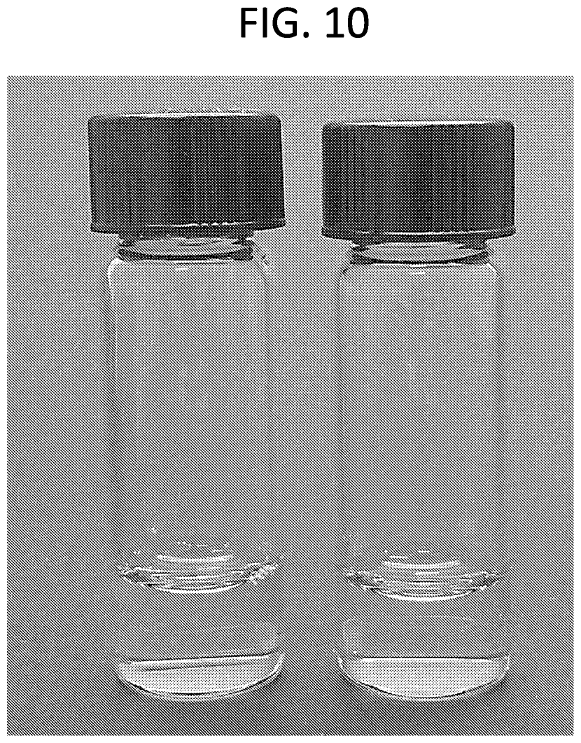

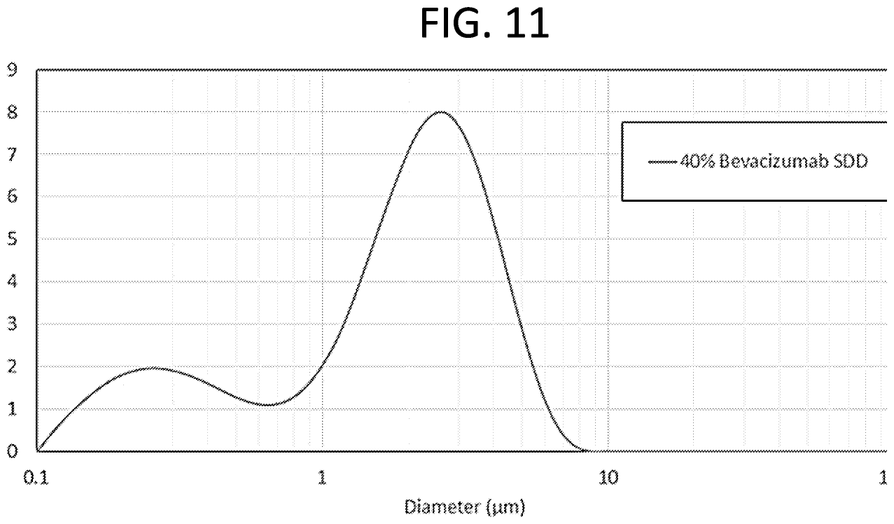

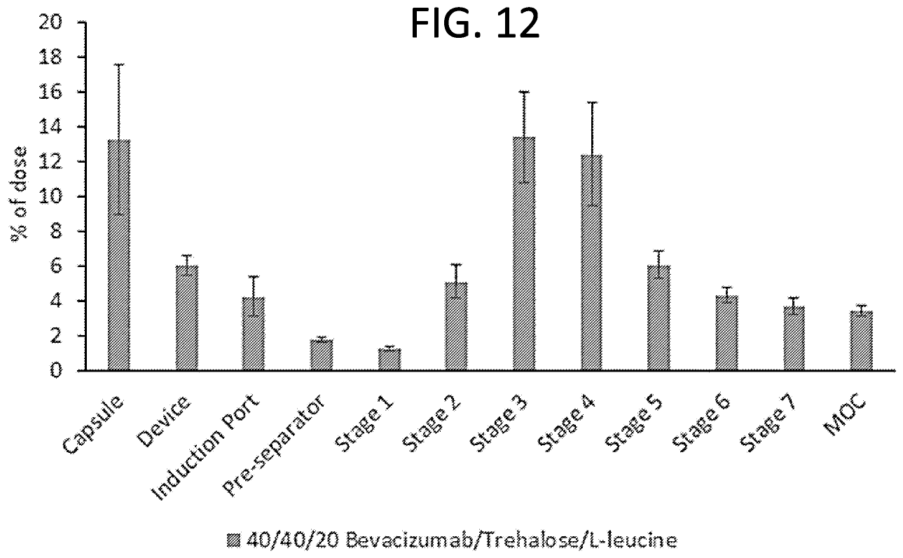

The L-leucine was crystalline by PXRD (FIG. 8), and the trehalose/mAb phase was amorphous by DSC. The aerodynamic particle size distribution was measured using a Next Generation Impactor. The morphology via SEM is represented in FIG. 9. Reconstituted SDDs solutions in buffer exhibit an identical appearance/transparency as the spray solution prior to spray drying (FIG. 10). The geometric particle size distribution by laser light scattering revealed a d50 value of 2.2 μm, a d90 value of 4.4 μm and a bimodal distribution with a minor peak below 500 nm (FIG. 11). The mass median aerodynamic diameter was 2.2 μm, the fine particle fraction was 81 wt % of the emitted dose, and the very fine particle fraction was 41 wt % of the emitted dose (FIG. 12). The fine particle fraction normalized by the capsule fill mass measured using a Fast Scanning Impactor was at 55 wt %.

The potency of the SDDs, measured by absorbance at 280 nm, was 37% bevacizumab.

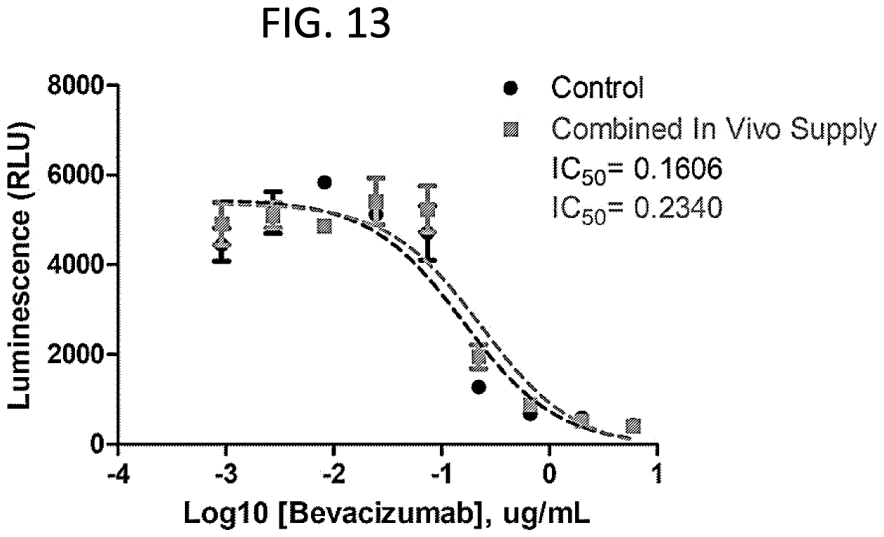

The SDD's biological activity to inhibit VEGF was measured using a Promega kit (see Example 1 for details) compared with a bevacizumab control. The IC50 for VEGF inhibition was 0.161 μg/mL for the SDD compared with 0.234 μg/mL for the control (FIG. 13).After a 3-month stability challenge at 25° C./60% RH (vial closed with desiccant), the SDD retained similar aerodynamic properties and biologic activity. After the stability challenge, the MMAD was 2.2 μm, FPF was 82 wt % and vFPF was 43 wt %. The anti-VEGF IC50 was 0.079 μg/mL for the SDD and 0.067 μg/mL for the control. The L-leucine remained crystalline and the trehalose/mAb phase remained amorphous as measured by PXRD.

SDDs of Examples 1, 3 and 4 were evaluated for physical stability, aerosol properties, and biological activity. Two physical-stability metrics were used: (1) the L-leucine phase in the SDD should be crystalline and (2) the amorphous trehalose/bevacizumab phase should have a high onset Tg.

All three formulations met these criteria, with PXRD peaks characteristic of crystalline spray-dried L-leucine, and onset Tgs of 117° C. Aerosol properties were measured using the NGI, with results shown in Table 3. The SDD of Example 4 met the MMAD specification (2 to 3 μm) and had the highest FPF value. Biological activity was tested via the activity assay described above. All three SDDs inhibited VEGF expression, with response curves statistically indistinguishable from the control's.

TABLE 3
Analytical results for of SDDs of Examples 1, 3 and 4.

| | Ex. 1 SDD | Ex. 3 SDD | Ex. 4 SDD |
|---|---|---|---|
| Onset T_{g} (° C.) | 117 | 117 | 117 |
| MMAD (μm) | 2.4 | 1.6 | 2.2 |
| FPF (%) | 66 | 73 | 82 |
| VEGF activity assay | 0.93 | 1.26 | 0.69 |
| (IC₅₀/IC_{50, control}) | | | |

An accelerated stability study was conducted. Samples of the three SDDs were tested before and after storage for 2 weeks in closed vials with desiccant at 40° C./75% relative humidity (RH). No significant changes were observed.

A real-time stability study was conducted with the bevacizumab SDD of Example 4, stored at two conditions: 5° C. and 25° C./60% RH. For these tests, 150 mg of SDD was sealed in a glass vial. The vial was heat-sealed in a Mylar® bag containing 2 g of silica gel desiccant. Samples were tested before and after storage for 1, 3, and 6 months. Overall, minimal changes were observed in the stability samples. Physical stability, potency and aerosol performance remained constant during storage (Table 4).

During storage, recrystallization of the amorphous components must be avoided. Thermal analysis properties of the SDD by DSC showed no melting phenomena, indicating that both trehalose and bevacizumab were amorphous. A broad Tg was observed in the reversing heat flow with an onset temperature of 118° C. and a midpoint temperature of 128° C., characteristic of the trehalose/bevacizumab homogeneous amorphous phase. Overall, thermal analysis indicated that the material is physically stable and has a low risk of failure during storage.

TABLE 4
Analytical results for of SDDs of Example
4 after 6 months storage at 25° C.

| | initial sample | | 6 months at 25° C. | |
|---|---|---|---|---|
| Tg Onset, ° C. | 117° | C. | 118° | C. |
| Bevacizumab potency | 37% | | 38% | |
| Fine particle fraction (by NGI) | 82% | | 84% | |
| MMAD (by NGI) | 2.2 | μm | 2.5 | μm |

| | | |
|---|---|---|
| Activity Assay | same as control | same as control |
| Water content | 3.0% | 3.7% |

### Example 5. In Vivo-Study of SDDs Comprising 40 wt % Bevacizumab/40 wt % Trehalose/20 wt % L-Leucine

An in vivo study of the SDDs obtained in Example 4 using an orthotopic nude rat model for lung cancer was designed to test the efficacy of inhaled bevacizumab SDD compared with inter-peritoneal bevacizumab, in combination with cisplatin. NSCLC cell line Calu-3 was instilled into the lungs of X-irradiated rats by an intratracheal route, targeting 1.5×10′ cells per installation. For the first four weeks of the study, no treatment was administered, allowing growth of the tumor cells. The study design is shown in Table 5. Inhaled bevacizumab SDD was evaluated as a primary treatment as well as a maintenance therapy.

During the primary treatment phase (weeks 4-8), Bevacizumab was administered via either intraperitoneal injection (IP) at 15 mg/kg once-weekly, or inhalation (INH) at 15 mg/kg presented dose, 1.5 mg/kg deposited dose once-weekly. Cisplatin was administered by IP at 3 mg/kg. Powder was aerosolized using a rotating brush generator and delivered to the rats passively through nasal inhalation. During the maintenance treatment phase (weeks 8-12), no additional cisplatin was administered, and only INH bevacizumab was administered once-weekly (15 mg/kg presented dose, 1.5 mg/kg deposited dose). Groups 1-4 were evaluated for primary efficacy after 8 weeks with lung weight as the end point. Groups 5-7 were evaluated for maintenance efficacy after 12 weeks with lung weight and survival as the end point.

TABLE 5
Design of in vivo efficacy study.

| | Primary Treatment | | Bevacizumab | | |
|---|---|---|---|---|---|
| | (Weeks 4-8) | | maintenance | | |
| | Cis- | | therapy | | |
| Group | platin | Bevacizumab | (Weeks 8-12) | Animals | End Point |
| 1 | No | No | No | 15 | 8 week |
| 2 | Yes | Yes (IP) | No | 15 | lung weight |
| 3 | No | Yes (INH) | No | 15 | |
| 4 | Yes | Yes (INH) | No | 15 | |
| 5 | No | No | No | 20 | 12 week |
| 6 | Yes | Yes (IP) | Yes (INH) | 15 | lung weight |
| 7 | Yes | Yes (INH) | Yes (INH) | 15 | and survival |

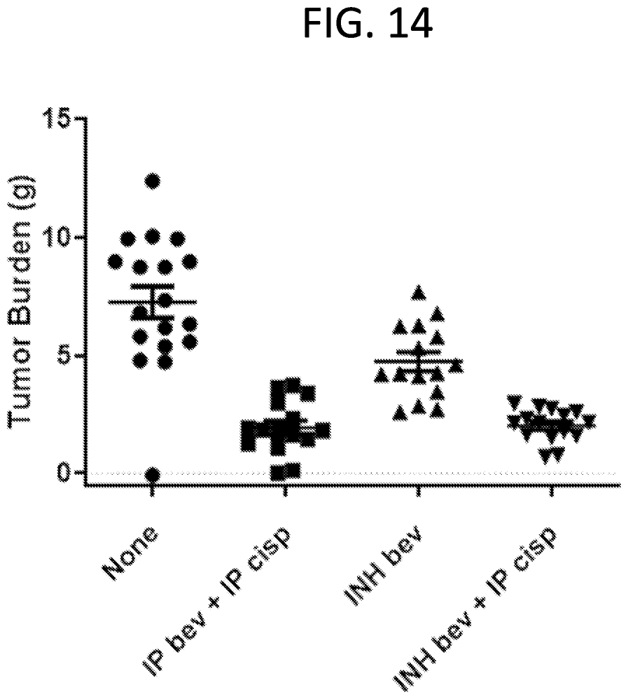

The results of the primary treatment phase of the study demonstrated that inhaled bevacizumab treatment significantly decreases lung weight (i.e. tumor burden) in the model rat system (Group 1 vs. Group 3; p<0.001). Treatment with cisplatin and bevacizumab in combination was more effective at reducing tumor burden than bevacizumab alone. Despite a 10-fold reduced dose for inhaled bevacizumab relative to IP bevacizumab (1.5 mg/kg vs. 15 mg/kg), the tumor burden for groups 2 and 4 was indistinguishable, and significantly lower than group 1 and 3. Tumor burden data is shown in FIG. 14.

Rats treated with the inhaled bevacizumab SDD alone had a statistically significant reduction in lung weight, characteristic of a reduced tumor burden, compared with the untreated group. Despite a 10-fold reduction in the dose for the inhaled bevacizumab SDD compared to bevacizumab delivered via IP injection (1.5 mg/kg deposited dose vs. 15 mg/kg IP injected dose), the reduction in tumor burden was equal when both were co-administered with cisplatin.

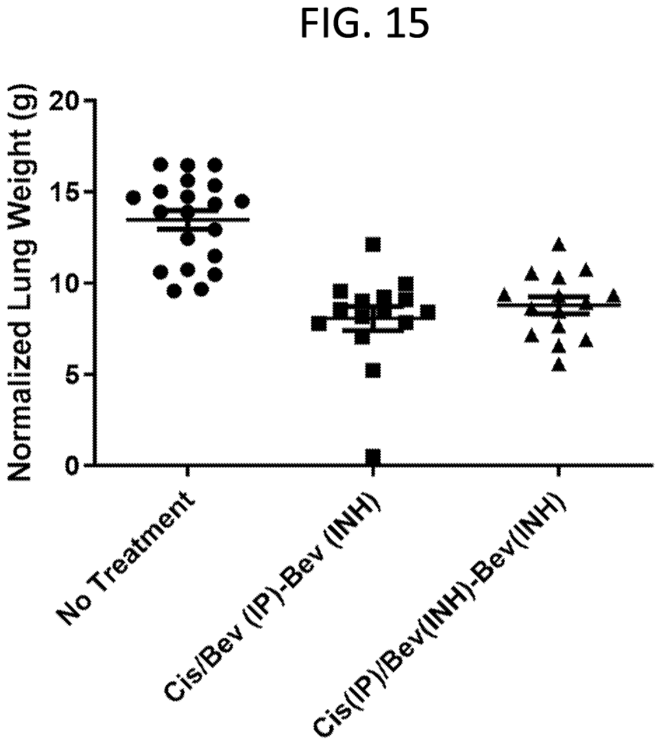

The maintenance phase of the study used 12-week lung weight and survival as end-points for the study. Inhaled bevacizumab was administered as the maintenance treatment for both group 6 and 7. In both cases, lung weight was significantly lower (p<0.001) than the group 5 control as shown in FIG. 15.

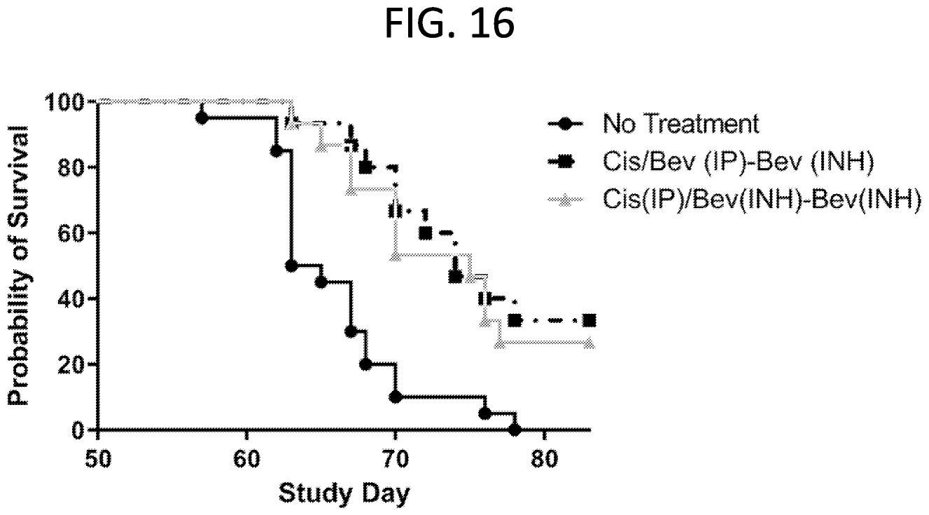

Additionally, individual rats survived longer in the maintenance phase of the study when in treatment group 6 or 7, compared with control as shown in FIG. 16.

### Example 6. Preparation of Fixed-Dose Combinations: Dual-API SDDs of Cisplatin:Bevacizumab (5 wt % Cisplatin/20 wt % Bevacizumab/55 wt % Trehalose/20 wt % L-Leucine)

A spray solution was prepared containing 0.5 mg/mL cisplatin, 2 mg/mL bevacizumab, 5.5 mg/mL trehalose and 2 mg/mL L-leucine in 1 mM phosphate buffer (pH 6.3). The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator. The materials were secondary dried overnight in a vacuum oven with nitrogen gas sweep at ambient temperature.

Potency analysis of the SDDs demonstrated a substantial change in the absorbance spectra for both bevacizumab and cisplatin, indicating that a chemical interaction had occurred between the active materials in the SDD. For this reason, a potency quantification could not be performed.

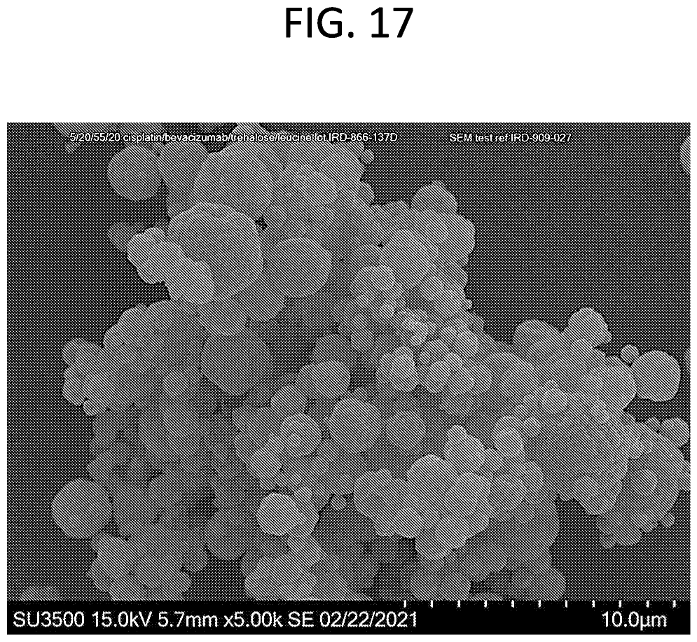

Analysis by PXRD indicated that the L-leucine is crystalline, and all other materials are amorphous. Thermal analysis by DSC confirmed that there are no melting peaks observed during a scan up to 160° C. An SEM image of the powder is provided in FIG. 17).

### Example 7. Preparation of Fixed-Dose Combinations: Dual-API SDDs of Cisplatin:Bevacizumab (10 wt % Cisplatin/20 wt % Bevacizumab/50 wt % Trehalose/20 wt % L-Leucine)

A spray solution was prepared containing 1 mg/mL cisplatin, 2 mg/mL bevacizumab, 5 mg/mL trehalose and 2 mg/mL L-leucine in 1 mM phosphate buffer (pH 6.3). The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator. The materials were secondary dried overnight in a vacuum oven with nitrogen gas sweep at ambient temperature.

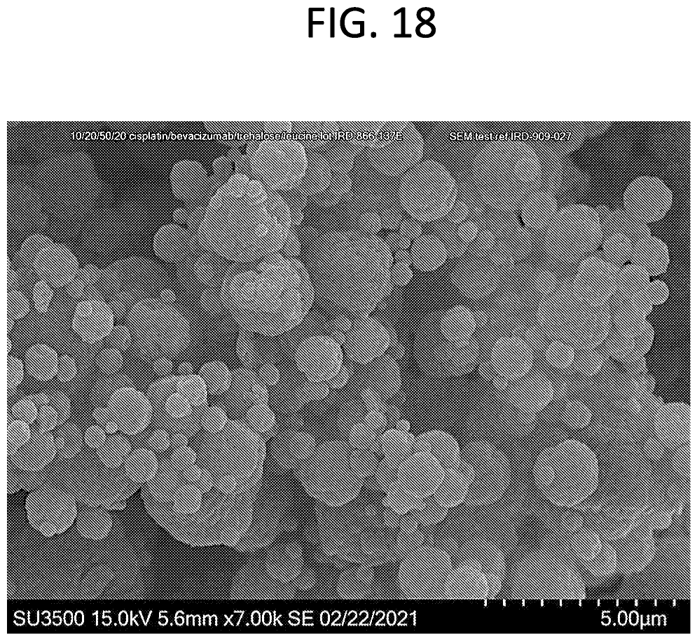

The potency of the active materials in the SDDs was quantified by absorbance at 9.1% cisplatin by weight and 18.4% bevacizumab by weight. The target potency was 10% cisplatin and 20% bevacizumab. Analysis by PXRD indicated that the L-leucine is crystalline, and all other materials are amorphous. Thermal analysis by DSC confirmed that there are no melting peaks observed during a scan up to 160° C. An SEM image of the powder is provided in FIG. 18).

### Example 8. Preparation of Fixed-Dose Combinations: Co-Sprayed Mono-API SDDs Erlotinib:Bevacizumab (Co-Spray Ratio 1:2) (80 wt % Erlotinib/20 wt % L-Leucine and 40 wt % Bevacizumab/40 wt % Trehalose/20 wt % L-Leucine)

A first spray drying solution was prepared containing 8 mg/mL erlotinib and 2 mg/mL L-leucine in a methanol-water mixture (9:1 by weight). A second spray drying solution was prepared containing 4 mg/mL bevacizumab, 4 mg/mL trehalose and 2 mg/mL L-leucine in 1 mM phosphate buffer (pH 6.3). The two spray solutions were pumped simultaneously into a pre-heated spray dryer equipped with two separate two-fluid nozzles (1650/64) on a single wand, with an inlet temperature of 110° C., outlet temperature of 50° C. and a drying gas flow rate of 500 g/min. The flow rate of the erlotinib solution was 2 g/min and the bevacizumab solution was 4 g/min, and the atomization pressure was 15 psi for both atomizers. The spray dried particles were collected by a 2″ cyclonic separator. The materials were secondary dried overnight in a vacuum oven with nitrogen gas sweep at ambient temperature.

The potency of the SDDs was measured by HPLC at 26.5% erlotinib and 24.4% bevacizumab. The target potency was 26.6% erlotinib and 26.6% bevacizumab. Analysis by PXRD indicated that both erlotinib and L-leucine are crystalline. A background amorphous halo was observed from the bevacizumab/trehalose phase. Thermal analysis by DSC confirmed the presence of crystalline erlotinib with a melt peak at ˜165° C.

Quantification of this peak indicated that the erlotinib in the SDD was 86% crystalline, and remainder amorphous. A glass transition temperature at 34° C. was also observed, characteristic of amorphous erlotinib. A broad Tg was also observed at ˜120° C., characteristic of the amorphous bevacizumab/trehalose phase.

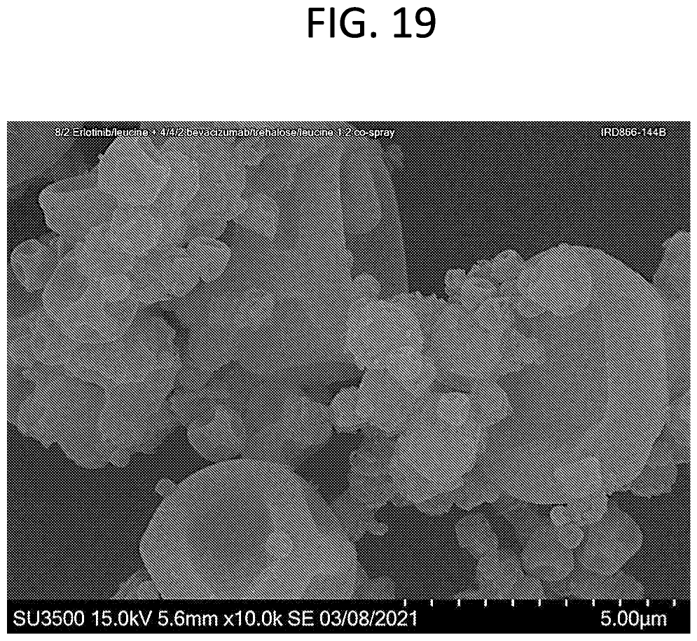

The aerodynamic particle size of the SDD was measured using a TSI Aerodynamic Particle Sizer, yielding a MMAD of 2.9 μm and geometric standard deviation (GSD) of 1.7 μm. The fine particle dose (FPD) was measured using a Fast Scanning Impactor, whereby the mass fraction of particles with an aerodynamic diameter of <5 μm were quantified gravimetrically. The fine particle dose (FPD) was normalized by the capsule fill mass (10 mg nominal) was 43.4%. An SEM image of the powder is provided in FIG. 19).

### Example 9. Preparation of Fixed-Dose Combinations: Co-Sprayed Mono-API SDDs Erlotinib:Bevacizumab (Co-Spray Ratio 1:1) (80 wt % Erlotinib/20 wt % L-Leucine and 40 wt % Bevacizumab/40 wt % Trehalose/20 wt % L-Leucine)

A first spray drying solution was prepared containing 8 mg/mL erlotinib and 2 mg/mL L-leucine in a methanol-water mixture (9:1 by weight). A second spray drying solution was prepared containing 4 mg/mL bevacizumab, 4 mg/mL trehalose and 2 mg/mL L-leucine in 1 mM phosphate buffer (pH 6.3). The two solutions were pumped simultaneously into a pre-heated spray dryer equipped with two separate two-fluid nozzles (1650/64) on a single wand, with an inlet temperature of 110° C., outlet temperature of 50° C. and a drying gas flow rate of 500 g/min. The flow rate of the erlotinib solution was 3 g/min and the bevacizumab solution was 3 g/min, and the atomization pressure was 20 psi for both atomizers. The spray dried particles were collected by a 2″ cyclonic separator. The materials were secondary dried overnight in a vacuum oven with nitrogen gas sweep at ambient temperature.

The potency of the SDDs was measured by HPLC at 41.7% erlotinib and 16.6% bevacizumab. The target potency was 40% erlotinib and 20% bevacizumab. Analysis by PXRD indicated that both erlotinib and L-leucine are crystalline. A background amorphous halo was observed from the bevacizumab/trehalose phase. Thermal analysis by DSC confirmed the presence of crystalline erlotinib with a melt peak at ˜165° C. Quantification of this peak indicated that the erlotinib in the SDD was 78% crystalline, and remainder amorphous. A glass transition temperature at 34° C. was also observed, characteristic of amorphous erlotinib. A broad Tg was also observed at ˜120° C., characteristic of the amorphous bevacizumab/trehalose phase.

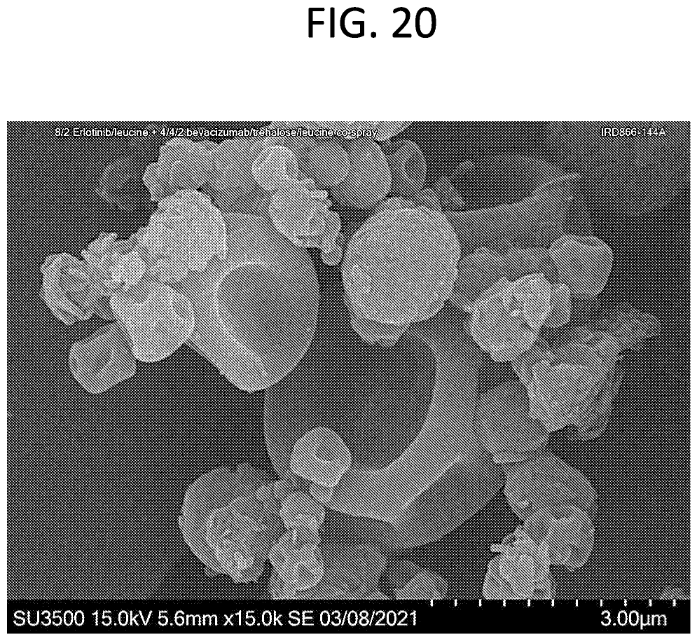

The aerodynamic particle size of the SDD was measured using an Aerodynamic Particle Sizer, yielding a MMAD of 2.5 μm and GSD of 1.7 μm. The fine particle dose was measured using a Fast Scanning Impactor, whereby the mass fraction of particles with an aerodynamic diameter of <5 μm were quantified gravimetrically. The fine particle dose normalized by the capsule fill mass (10 mg nominal) was 46.3%. An SEM image of the powder (FIG. 20) showed clear morphology differences between the two types of particles. Bevacizumab SDDs are wrinkled with a smooth surface, while erlotinib SDDs are more spherical with a rough surface.

### Example 10. Preparation of Fixed-Dose Combinations: Co-Sprayed Mono-API SDDs Paclitaxel:Bevacizumab (Co-Spray Ratio 1:5) (80 wt % Paclitaxel/20 wt % L-Leucine and 40 wt % Bevacizumab/40 wt % Trehalose/20 wt % L-Leucine)

A solution of 6 mg/mL paclitaxel and 1.5 mg/mL L-leucine was prepared in 80/20 ethanol/water by weight. A second solution of 4 mg/mL bevacizumab, 4 mg/mL trehalose and 2 mg/mL L-leucine was prepared in pH 6.3 1 mM phosphate buffer. The two solutions were pumped simultaneously into a spray dryer equipped with two separate two-fluid nozzles (1650/64) on a single wand. The spray solutions were spray dried in a pre-heated spray dryer with an inlet temperature of 110° C., outlet temperature of 50° C. and a drying gas flow rate of 500 g/min. The flow rate of the paclitaxel solution was 1.5 g/min and the bevacizumab solution was 5.0 g/min, and the atomization pressure was 20 psi for the bevacizumab solution and 15 psi for the paclitaxel solution. The spray dried particles were collected by a 2″ cyclonic separator. The materials were secondary dried overnight in a vacuum oven with nitrogen gas sweep at ambient temperature.

The potency of the SDDs was measured by UV absorbance at 12±1% paclitaxel and 32±0.3% bevacizumab. The target potency was 13.3% paclitaxel and 33.3% bevacizumab. Analysis by PXRD indicated that the L-leucine is crystalline, and all other materials are amorphous. Thermal analysis by DSC confirmed that there were no melting peaks observed during a scan up to 160° C. One glass transition temperatures could be resolved: a broad transition at 118° C. onset characteristic of the bevacizumab SDD. Due to the low paclitaxel content, the expected transition was subtle at ˜150° C. and could not be quantified.

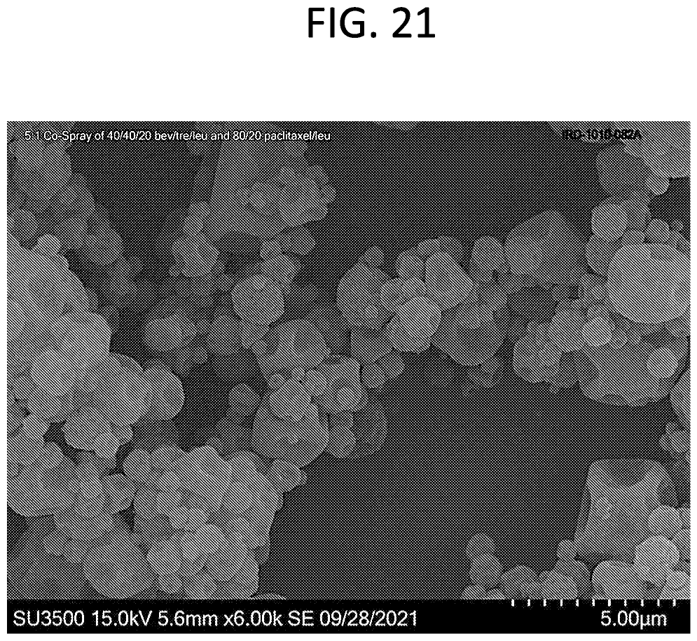

The aerodynamic particle size of the SDD was measured using a TSI Aerodynamic Particle Sizer, yielding a MMAD of 2.1 μm and GSD of 1.6 μm. The fine particle dose was measured using a fast-screening impactor, whereby the mass fraction of particles with an aerodynamic diameter of <5 μm were quantified gravimetrically. The fine particle dose normalized by the capsule fill mass (10 mg nominal) was 64.3%. An SEM image of the powder (FIG. 21) showed clear morphology differences between the two types of particles. Bevacizumab SDDs are wrinkled with a smooth surface, while paclitaxel SDDs are more spherical with a rough surface.

### Example 11. Preparation of Fixed-Dose Combinations: Co-Sprayed Mono-API SDDs Paclitaxel:Bevacizumab (Co-Spray Ratio 1:2) (80 wt % Paclitaxel/20 wt % L-Leucine and 40 wt % Bevacizumab/40 wt % Trehalose/20 wt % L-Leucine)

A solution of 6 mg/mL paclitaxel and 1.5 mg/mL L-leucine was prepared in 80/20 ethanol/water by weight. A second solution of 4 mg/mL bevacizumab, 4 mg/mL trehalose and 2 mg/mL L-leucine was prepared in pH 6.3 1 mM phosphate buffer. The two solutions were pumped simultaneously into a spray dryer equipped with two separate two-fluid nozzles (1650/64) on a single wand. The spray solutions were spray dried in a pre-heated spray dryer with an inlet temperature of 110° C., outlet temperature of 50° C. and a drying gas flow rate of 500 g/min. The flow rate of the paclitaxel solution was 3.0 g/min and the bevacizumab solution was 4.0 g/min, and the atomization pressure was 20 psi for the bevacizumab solution and 15 psi for the paclitaxel solution. The spray dried particles were collected by a 2″ cyclonic separator. The materials were secondary dried overnight in a vacuum oven with nitrogen gas sweep at ambient temperature.

The potency of the SDDs was measured by UV absorbance at 24±2% paclitaxel and 27±1% bevacizumab. The target potency was 26.7% paclitaxel and 26.7% bevacizumab. Analysis by PXRD indicated that the L-leucine is crystalline, and all other materials are amorphous. Thermal analysis by DSC confirmed that there were no melting peaks observed during a scan up to 160° C. Two separate glass transition temperatures could be resolved, a broad transition onset at 118° C. characteristic of the bevacizumab SDD, and a transition at 150° C. onset characteristic of pure amorphous paclitaxel.

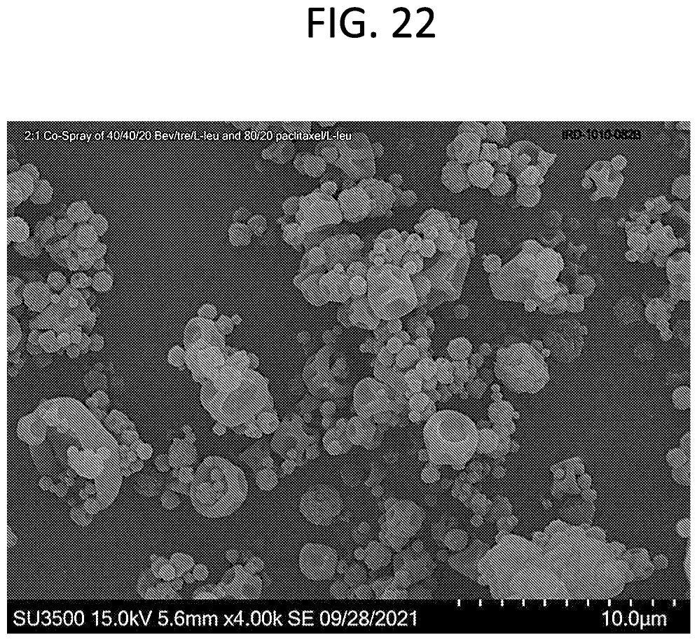

The aerodynamic particle size of the SDD was measured using a TSI Aerodynamic Particle Sizer, yielding a MMAD of 2.0 μm and GSD of 1.7 μm. The fine particle dose was measured using a fast-screening impactor, whereby the mass fraction of particles with an aerodynamic diameter of <5 μm were quantified gravimetrically. The fine particle dose normalized by the capsule fill mass (10 mg nominal) was 34.0%. An SEM image of the powder (FIG. 22) showed clear morphology differences between the two types of particles. Bevacizumab SDDs are wrinkled with a smooth surface, while paclitaxel SDDs are more spherical with a rough surface.

### Example 12. Preparation of Fixed-Dose Combinations: Co-Sprayed Mono-API SDDs Paclitaxel:Bevacizumab (Co-Spray Ratio 1:1) (80 wt % Paclitaxel/20 wt % L-Leucine and 40 wt % Bevacizumab/40 wt % Trehalose/20 wt % L-Leucine)

A first spray drying solution was prepared containing 6 mg/mL paclitaxel and 1.5 mg/mL L-leucine in a ethanol-water mixture (4:1 by weight). A second spray drying solution was prepared containing 4 mg/mL bevacizumab, 4 mg/mL trehalose and 2 mg/mL L-leucine in 1 mM phosphate buffer (pH 6.3). The two solutions were pumped simultaneously into a pre-heated spray dryer spray dryer equipped with two separate two-fluid nozzles (1650/64) on a single wand, with an inlet temperature of 110° C., outlet temperature of 50° C. and a drying gas flow rate of 500 g/min. The flow rate of the paclitaxel solution was 3.4 g/min and the bevacizumab solution was 2.6 g/min, and the atomization pressure was 15 psi for both atomizers. The spray dried particles were collected by a 2″ cyclonic separator. The materials were secondary dried overnight in a vacuum oven with nitrogen gas sweep at ambient temperature.

The potency of the SDDs was measured by HPLC at 41.9% paclitaxel and 19.5% bevacizumab. The target potency was 40% paclitaxel and 20% bevacizumab. Analysis by PXRD indicated that the L-leucine is crystalline, and all other materials are amorphous. Thermal analysis by DSC confirmed that there were no melting peaks observed during a scan up to 160° C. Two separate glass transition temperatures could be resolved, a broad transition at 118° C. characteristic of the bevacizumab SDD, and a transition at ˜150° C. characteristic of pure amorphous paclitaxel.

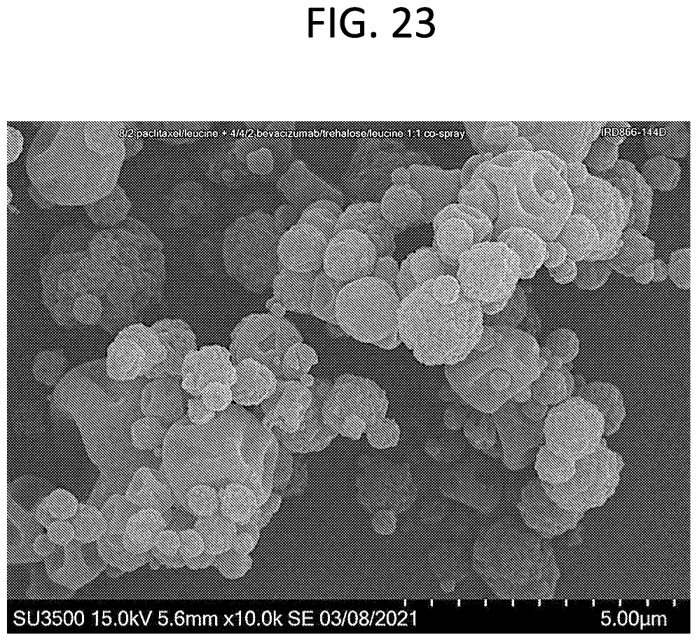

The aerodynamic particle size of the SDD was measured using an Aerodynamic Particle Sizer, yielding a MMAD of 2.4 μm and GSD of 1.7 μm. The fine particle dose was measured using a Fast Scanning Impactor, whereby the mass fraction of particles with an aerodynamic diameter of <5 μm were quantified gravimetrically. The fine particle dose normalized by the capsule fill mass (10 mg nominal) was 68.6%. An SEM image of the powder (FIG. 23) showed clear morphology differences between the two types of particles. Bevacizumab SDDs are wrinkled with a smooth surface, while paclitaxel SDDs are more spherical with a rough surface.

### Example 13. Preparation of Fixed-Dose Combinations: Co-Sprayed Mono-API SDDs Paclitaxel:Bevacizumab (Co-Spray Ratio 2:1) (80 wt % Paclitaxel/20 wt % L-Leucine and 40 wt % Bevacizumab/40 wt % Trehalose/20 wt % L-Leucine)

A solution of 6 mg/mL paclitaxel and 1.5 mg/mL L-leucine was prepared in 80/20 ethanol/water by weight. A second solution of 4 mg/mL bevacizumab, 4 mg/mL trehalose and 2 mg/mL L-leucine was prepared in pH 6.3 1 mM phosphate buffer. The two solutions were pumped simultaneously into a spray dryer equipped with two separate two-fluid nozzles (1650/64) on a single wand. The spray solutions were spray dried in a pre-heated spray dryer with an inlet temperature of 110° C., outlet temperature of 50° C. and a drying gas flow rate of 500 g/min. The flow rate of the paclitaxel solution was 6.1 g/min and the bevacizumab solution was 2.0 g/min, and the atomization pressure was 20 psi for the bevacizumab solution and 15 psi for the paclitaxel solution. The spray dried particles were collected by a 2″ cyclonic separator. The materials were secondary dried overnight in a vacuum oven with nitrogen gas sweep at ambient temperature.

The potency of the SDDs was measured by UV absorbance at 52±4% paclitaxel and 13±1% bevacizumab. The target potency was 53.3% paclitaxel and 13.3% bevacizumab. Analysis by PXRD indicated that the L-leucine is crystalline, and all other materials are amorphous. Thermal analysis by DSC confirmed that there were no melting peaks observed during a scan up to 160° C. Two separate glass transition temperatures could be resolved, a broad transition at 118° C. onset characteristic of the bevacizumab SDD, and a transition at 150° C. onset characteristic of pure amorphous paclitaxel.

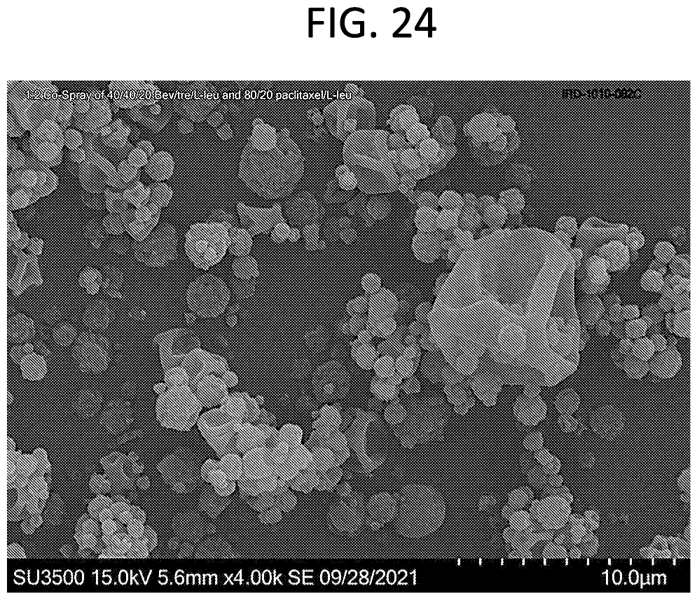

The aerodynamic particle size of the SDD was measured using a TSI Aerodynamic Particle Sizer, yielding a MMAD of 1.6 μm and GSD of 1.6 μm. The fine particle dose was measured using a fast-screening impactor, whereby the mass fraction of particles with an aerodynamic diameter of <5 μm were quantified gravimetrically. The fine particle dose normalized by the capsule fill mass (10 mg nominal) was 65.2%. An SEM image of the powder (FIG. 24) showed clear morphology differences between the two types of particles. Bevacizumab SDDs are wrinkled with a smooth surface, while paclitaxel SDDs are more spherical with a rough surface.

### Example 14. Preparation of Fixed-Dose Combinations: Co-Sprayed Mono-API SDDs Paclitaxel:Bevacizumab (Co-Spray Ratio 5:1) (80 wt % Paclitaxel/20 wt % L-Leucine and 40 wt % Bevacizumab/40 wt % Trehalose/20 wt % L-Leucine)

A solution of 6 mg/mL paclitaxel and 1.5 mg/mL L-leucine was prepared in 80/20 ethanol/water by weight. A second solution of 4 mg/mL bevacizumab, 4 mg/mL trehalose and 2 mg/mL L-leucine was prepared in pH 6.3 1 mM phosphate buffer. The two solutions were pumped simultaneously into a spray dryer equipped with two separate two-fluid nozzles (1650/64) on a single wand. The spray solutions were spray dried in a pre-heated spray dryer with an inlet temperature of 110° C., outlet temperature of 50° C. and a drying gas flow rate of 500 g/min. The flow rate of the paclitaxel solution was 7.7 g/min and the bevacizumab solution was 1.0 g/min, and the atomization pressure was 20 psi for the bevacizumab solution and 15 psi for the paclitaxel solution. The spray dried particles were collected by a 2″ cyclonic separator. The materials were secondary dried overnight in a vacuum oven with nitrogen gas sweep at ambient temperature.

The potency of the SDDs was measured by UV absorbance at 65±1% paclitaxel and 10±2% bevacizumab. The target potency was 66.7% paclitaxel and 6.7% bevacizumab. Analysis by PXRD indicated that the L-leucine is crystalline, and all other materials are amorphous. Thermal analysis by DSC confirmed that there were no melting peaks observed during a scan up to 160° C. Two separate glass transition temperatures could be resolved, a weak transition onset at 118° C. characteristic of the bevacizumab SDD, and a transition at 150° C. characteristic of pure amorphous paclitaxel.

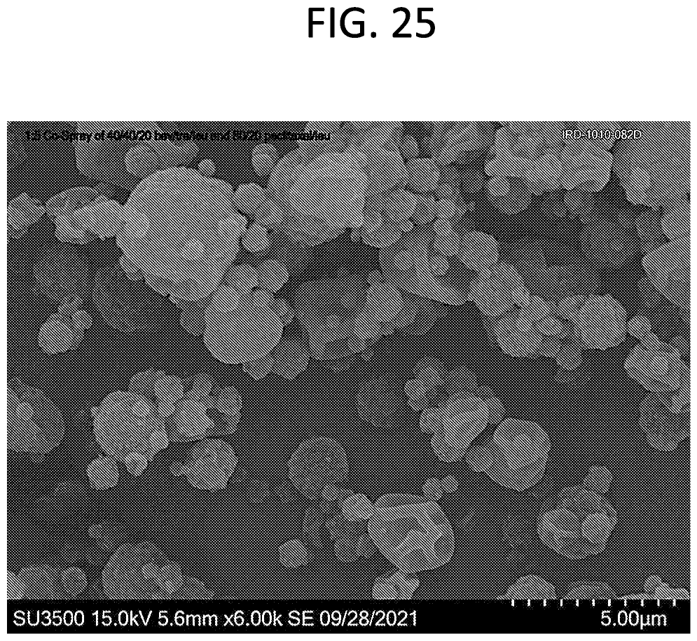

The aerodynamic particle size of the SDD was measured using a TSI Aerodynamic Particle Sizer, yielding a MMAD of 1.7 μm and GSD of 1.5 μm. The fine particle dose was measured using a fast-screening impactor, whereby the mass fraction of particles with an aerodynamic diameter of <5 μm were quantified gravimetrically. The fine particle dose normalized by the capsule fill mass (10 mg nominal) was 65.1%. An SEM image of the powder particle dose normalized by the capsule fill mass (10 mg nominal) was 65.2%. An SEM image of the powder (FIG. 25) showed clear morphology differences between the two types of particles. Bevacizumab SDDs are wrinkled with a smooth surface, while paclitaxel SDDs are more spherical with a rough surface.

### Example 15. Preparation of Fixed-Dose Combinations: Co-Sprayed Mono-API SDDs Cisplatin:Bevacizumab (Co-Spray Ratio 2:1) (10 wt % Cisplatin/70 wt % Trehalose/20 wt % L-Leucine and 40 wt % Bevacizumab/40 wt % Trehalose/20 wt % L-Leucine)

A first spray drying solution was prepared containing 1 mg/mL cisplatin, 7 mg/mL trehalose, and 2 mg/mL L-leucine in water. A second spray drying solution was prepared containing 4 mg/mL bevacizumab, 4 mg/mL trehalose and 2 mg/mL L-leucine in 1 mM phosphate buffer (pH 6.3). The two solutions were pumped simultaneously into a pre-heated spray dryer equipped with two separate two-fluid nozzles (1650/64) on a single wand, with an inlet temperature of 110° C., outlet temperature of 50° C. and a drying gas flow rate of 500 g/min. The flow rate of the cisplatin solution was 4 g/min and the bevacizumab solution was 2 g/min, and the atomization pressure was 15 psi for both atomizers. The spray dried particles were collected by a 2″ cyclonic separator. The materials were secondary dried overnight in a vacuum oven with nitrogen gas sweep at ambient temperature.

The cisplatin solution was prepared 18 hours in advance of the spray drying to allow adequate time for the slow-dissolving API to go into solution. However, it was later found that cisplatin converts to transplatin in aqueous solution on this time scale, reducing the potency. The potency of the SDDs was measured by UV-Vis absorbance at 4.7% cisplatin and 13.4% bevacizumab. The target potency was 6.7% cisplatin and 13.3% bevacizumab. Analysis by PXRD indicated that the L-leucine is crystalline, and all other materials are amorphous. Thermal analysis by DSC confirmed that there are no melting peaks observed during a scan up to 160° C. and the presence of a broad glass transition temperature at ˜110° C., confirming the presence of amorphous material. The individual Tgs of the amorphous bevacizumab/trehalose and cisplatin/trehalose phases overlap one another and cannot be distinguished separately.

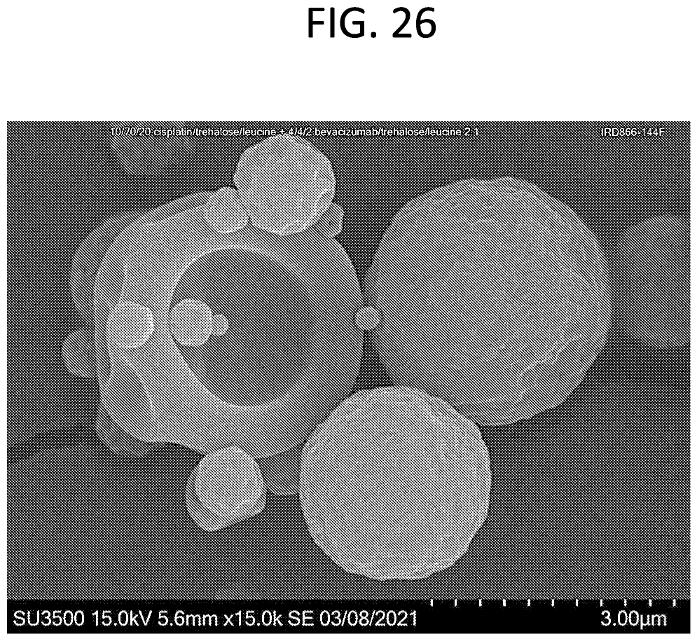

The aerodynamic particle size of the SDD was measured using an Aerodynamic Particle Sizer, yielding a MMAD of 3.0 μm and GSD of 1.7 μm. The fine particle dose was measured using a Fast Scanning Impactor, whereby the mass fraction of particles with an aerodynamic diameter of <5 μm were quantified gravimetrically. The fine particle dose normalized by the capsule fill mass (10 mg nominal) was 56.0%. An SEM image of the powder is provided in FIG. 26).

### Example 16. Preparation of Fixed-Dose Combinations: Co-Sprayed Mono-API SDDs Cisplatin:Bevacizumab (Co-Spray Ratio 1:1) (10 wt % Cisplatin/70 wt % Trehalose/20 wt % L-Leucine and 40 wt % Bevacizumab/40 wt % Trehalose/20 wt % L-Leucine)

A first spray drying solution was prepared containing 1 mg/mL cisplatin, 7 mg/mL trehalose, and 2 mg/mL L-leucine in water. A second spray drying solution was prepared containing 4 mg/mL bevacizumab, 4 mg/mL trehalose and 2 mg/mL L-leucine in 1 mM phosphate buffer (pH 6.3). The two solutions were pumped simultaneously into a pre-heated spray dryer equipped with two separate two-fluid nozzles (1650/64) on a single wand, with an inlet temperature of 110° C., outlet temperature of 50° C. and a drying gas flow rate of 500 g/min. The flow rate of the cisplatin solution was 3 g/min and the bevacizumab solution was 3 g/min, and the atomization pressure was 15 psi for both atomizers. The spray dried particles were collected by a 2″ cyclonic separator. The materials were secondary dried overnight in a vacuum oven with nitrogen gas sweep at ambient temperature.

The cisplatin solution was prepared 18 hours in advance of the spray drying to allow adequate time for the slow-dissolving API to go into solution. However, it was later found in the literature that cisplatin converts to transplatin in aqueous solution on this time scale, reducing the potency. The potency of the SDDs was measured by UV-Vis absorbance at 3.7% cisplatin and 19.7% bevacizumab. The target potency was 5% cisplatin and 20% bevacizumab. Analysis by PXRD indicated that the L-leucine is crystalline, and all other materials are amorphous. Thermal analysis by DSC confirmed that there are no melting peaks observed during a scan up to 160° C. and the presence of a broad glass transition temperature at ˜110° C., confirming the presence of amorphous material. The individual Tgs of the amorphous bevacizumab/trehalose and cisplatin/trehalose phases overlap one another and cannot be distinguished separately.

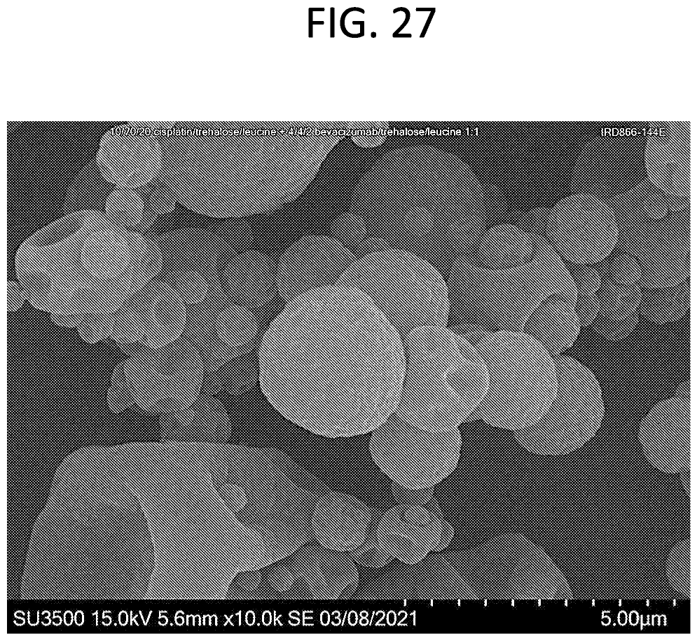

The aerodynamic particle size of the SDD was measured using an Aerodynamic Particle Sizer, yielding a MMAD of 2.8 μm and GSD of 1.7 μm. The fine particle dose was measured using a Fast Scanning Impactor, whereby the mass fraction of particles with an aerodynamic diameter of <5 μm were quantified gravimetrically. The fine particle dose normalized by the capsule fill mass (10 mg nominal) was 57.6%. An SEM image of the powder (FIG. 27) showed clear morphology differences between the two types of particles. Bevacizumab SDDs are wrinkled with a smooth surface, while cisplatin SDDs are more spherical with a rough surface.

### Example 17. Preparation of SDDs Comprising 40 wt % Bevacizumab/40 wt % Mannitol/20 wt % L-Leucine

A spray solution was prepared containing 4 mg/mL bevacizumab, 4 mg/mL mannitol and 2 mg/mL L-leucine in pH 6.3 1 mM phosphate buffer. The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator.

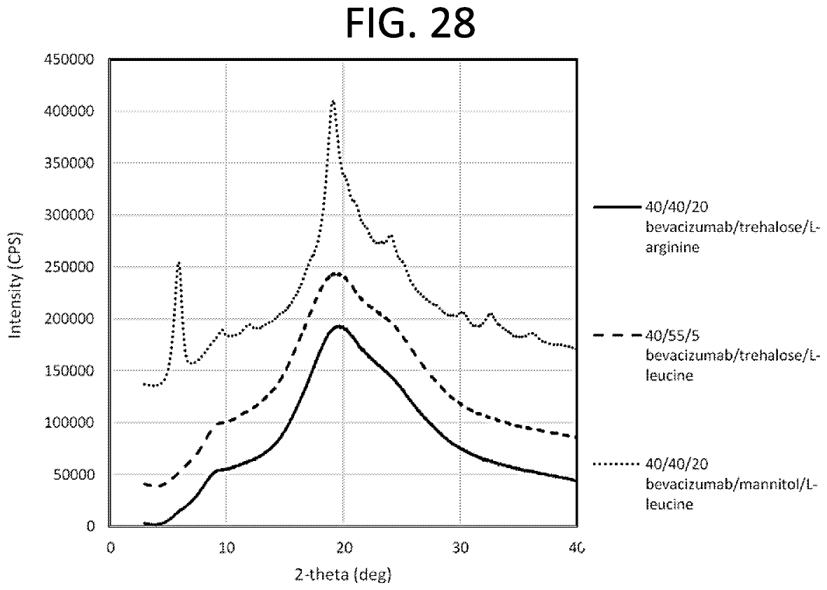

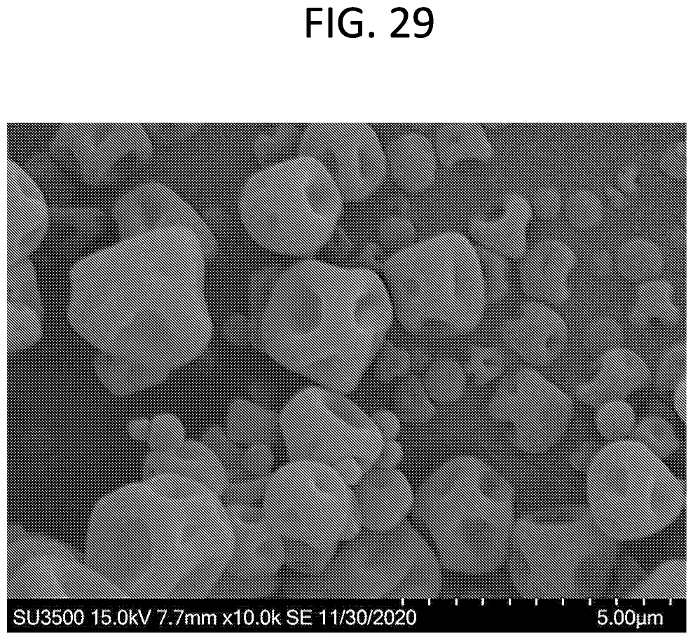

The L-leucine was crystalline by PXRD, and no peaks characteristic of crystalline mannitol were identified (FIG. 28). Thermal analysis by DSC showed multiple phases in the material, including a low-Tg phase near 14° C., characteristic of a mannitol-rich phase. A second, higher Tg was observed near 135° C., characteristic of a bevacizumab-rich phase. Bevacizumab melting was observed immediately after the second Tg. The morphology via SEM is represented in FIG. 29. The fine particle fraction normalized by the capsule fill mass measured using a Fast Scanning Impactor was at 43 wt %.

### Example 18. Preparation of SDDs Comprising 40 wt % Bevacizumab/55 wt % Trehalose/5 wt % L-Leucine

A spray solution was prepared containing 4 mg/mL bevacizumab, 5.5 mg/mL trehalose and 0.5 mg/mL L-leucine in pH 6.3 1 mM phosphate buffer. The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator.

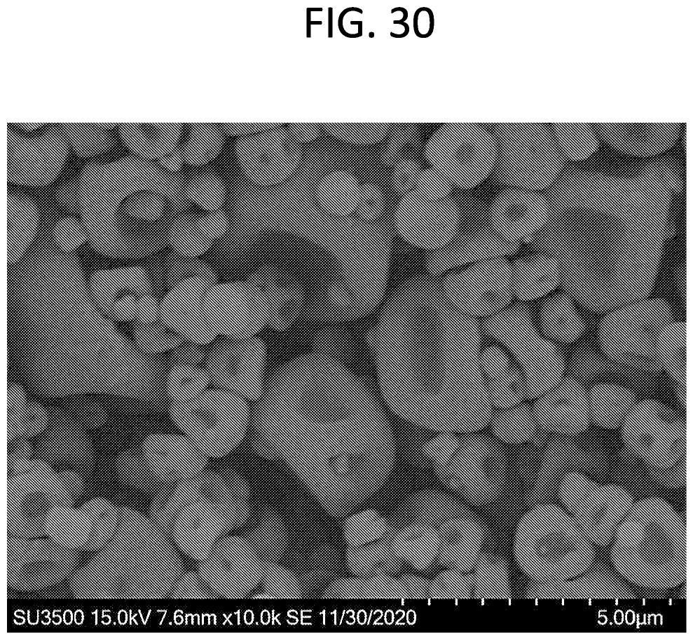

The SDD was amorphous by PXRD, with no characteristic peaks of L-leucine observed (FIG. 28). Thermal analysis by DSC showed a single amorphous phase, with a Tg onset at 111° C. The morphology via SEM is represented in FIG. 30. The fine particle fraction normalized by the capsule fill mass measured using a Fast Scanning Impactor was at 35 wt %.

### Example 19. Preparation of SDDs Comprising 40 wt % Bevacizumab/40 wt % Trehalose/20 wt % L-Arginine

A spray solution was prepared containing 4 mg/mL bevacizumab, 4 mg/mL trehalose and 2 mg/mL L-arginine in pH 6.3 1 mM phosphate buffer. The spray solutions were spray dried in a pre-heated spray dryer at a solution feed rate of 6 g/min, an inlet temperature of 120° C., outlet temperature of 50° C. and a drying gas flow rate of 500 g/min. The solution was atomized through a two-fluid nozzle (1650/64) at a pressure of 25 psi. The spray dried particles were collected by a 2″ cyclonic separator.

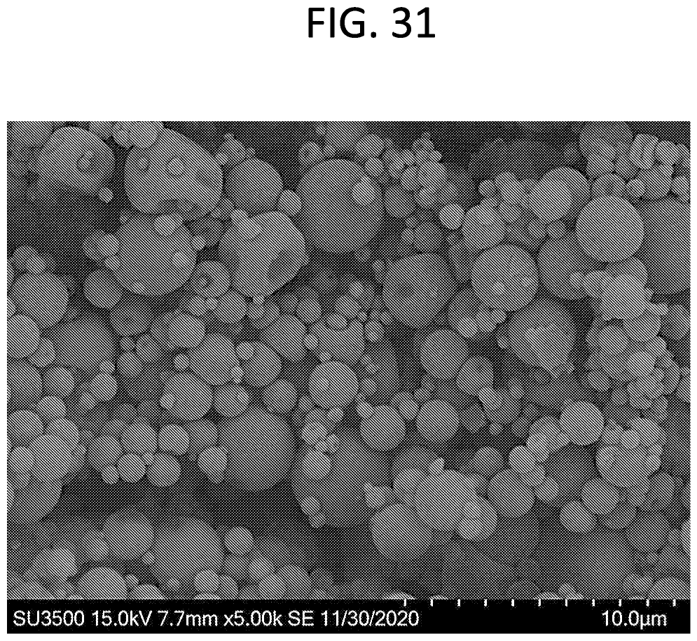

The SDD was amorphous by PXRD (FIG. 28). Thermal analysis by DSC showed a single amorphous phase, with a Tg onset at 106° C. The morphology via SEM is represented in FIG. 31. The fine particle fraction normalized by the capsule fill mass measured using a Fast Scanning Impactor was at 28 wt %.

### Example 20. Preparation of SDDs Comprising 40 wt % Bevacizumab/40 wt % Trehalose/20 wt % Trileucine

Initially, a spray solution was prepared containing 4 mg/mL bevacizumab, 4 mg/mL trehalose and 2 mg/mL L-leucine in pH 6.3 1 mM phosphate buffer. It was found that substantial undissolved trileucine was present in this solution after 2 hours of stirring, so it was diluted 1:1 with additional phosphate buffer. The spray solutions were spray dried in a pre-heated spray dryer at a solution feed rate of 10 g/min, an inlet temperature of 95-105° C., outlet temperature of 50° C. and a drying gas flow rate of 550 g/min. The solution was atomized through a two-fluid nozzle (1650/64) at a pressure of 25 psi. The spray dried particles were collected by a 2″ cyclonic separator.

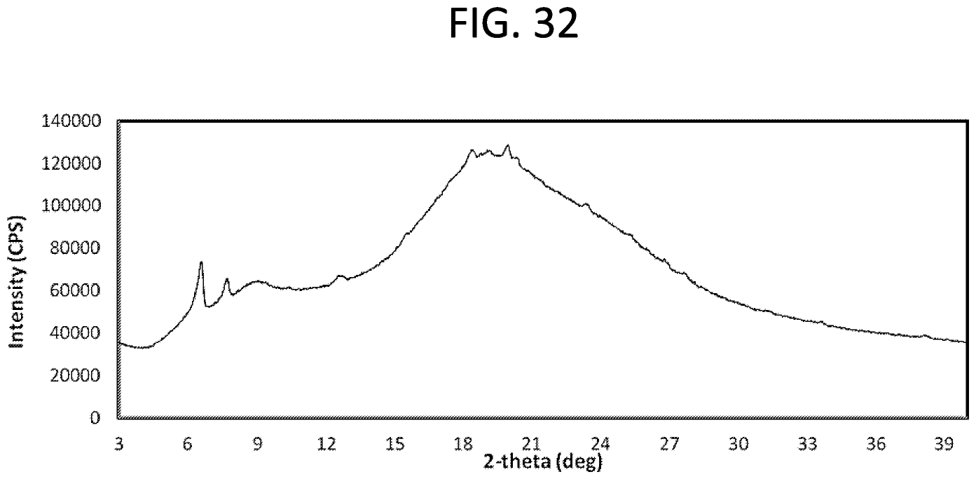

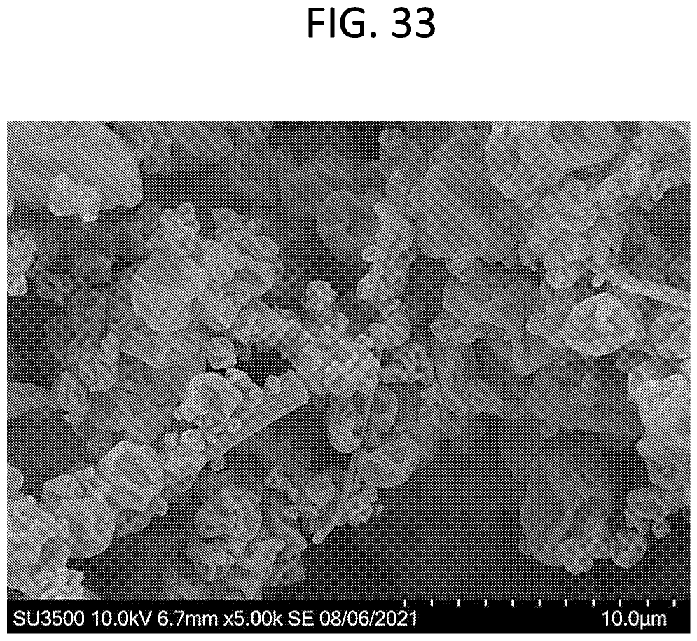

The SDD was predominantly amorphous by PXRD, with only weak peaks characteristic of trileucine (FIG. 32). Thermal analysis by DSC showed a single amorphous phase, with a Tg midpoint of 128° C. The morphology via SEM is shown in FIG. 33, and showed evidence of undissolved particles, which are likely trileucine. The fine particle fraction normalized by the capsule fill mass measured using a Fast Scanning Impactor was 67±6.7 wt %. The material was 6.6 wt % water measured by Karl Fisher titration.

### Example 21. Preparation of SDDs Comprising 40 wt % Bevacizumab/35 wt % Trehalose/20 wt % Trileucine/5 wt % Histidine

Initially, a spray solution was prepared containing 4 mg/mL bevacizumab, 4 mg/mL trehalose and 2 mg/mL L-leucine in 0.5 mg/mL pH 5.3 histidine buffer. It was found that substantial undissolved trileucine was present in this solution after 2 hours of stirring, so it was diluted 1:1 with additional histidine buffer. The material still did not fully dissolve, and so the supernatant was spray dried. The spray solutions were spray dried in a pre-heated spray dryer at a solution feed rate of 10 g/min, an inlet temperature of 95-105° C., outlet temperature of 50° C. and a drying gas flow rate of 550 g/min. The solution was atomized through a two-fluid nozzle (1650/64) at a pressure of 25 psi. The spray dried particles were collected by a 2″ cyclonic separator.

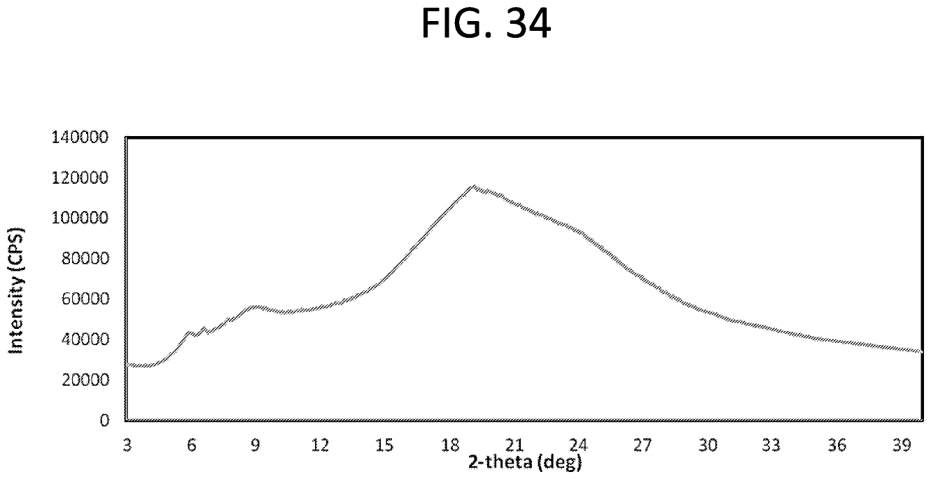

The SDD was predominantly amorphous by PXRD, with only weak peaks characteristic of trileucine (FIG. 34). Thermal analysis by DSC showed a very broad Tg with onset of 106° C. and endset of 137° C. This breadth likely indicates the presence of multiple amorphous phases with similar glass transition temperatures, suggesting a phase separated morphology. The fine particle fraction normalized by the capsule fill mass measured using a Fast Scanning Impactor was 66±8.7 wt %, with unusually high scatter in the measurements. The material was 6.1 wt % water measured by Karl Fisher titration.

### Example 22. Preparation of SDDs Comprising 25 wt % Bevacizumab/25 wt % Trehalose/50 wt % L-Leucine

A spray solution was prepared containing 2.5 mg/mL bevacizumab, 2.5 mg/mL trehalose and 5 mg/mL L-leucine in pH 6.3 1 mM phosphate buffer. The spray solutions were spray dried in a pre-heated spray dryer at a solution feed rate of 10 g/min, an inlet temperature of 95-105° C., outlet temperature of 50° C. and a drying gas flow rate of 550 g/min. The solution was atomized through a two-fluid nozzle (1650/64) at a pressure of 25 psi. The spray dried particles were collected by a 2″ cyclonic separator.

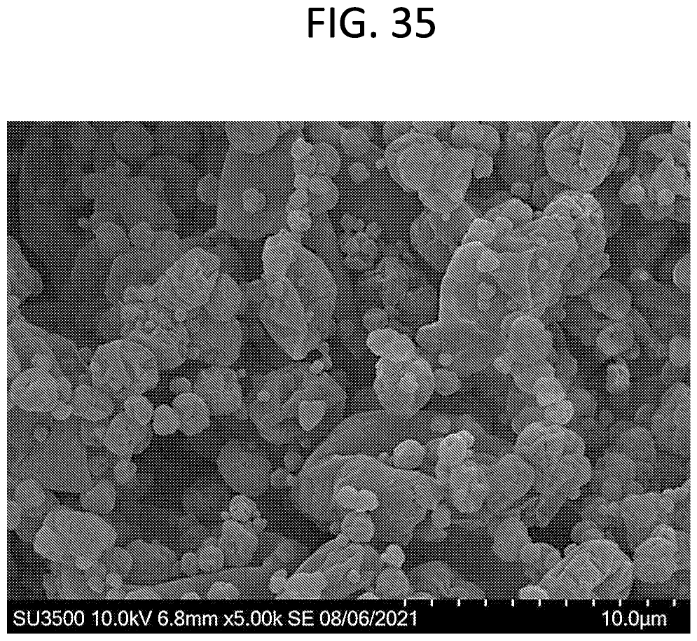

The L-leucine in the SDD was crystalline by PXRD. Thermal analysis by DSC showed very weak, inconsistent transitions which could not be quantified in the range of temperatures expected for the glass transition of bevacizumab/trehalose mixtures (100-140° C.). The morphology via SEM is shown in FIG. 35. The fine particle fraction normalized by the capsule fill mass measured using a Fast Scanning Impactor was 64±4.9 wt %. The material was 3.7 wt % water measured by Karl Fisher titration, and potency measured by A280 absorbance was 24.1% bevacizumab on a dry basis.

### Example 23. Preparation of SDDs Comprising 4 wt % Bevacizumab/85.5 wt % Trehalose/10 wt % L-Leucine/0.5 wt % Phosphate Buffer

A spray solution was prepared containing 0.4 mg/mL bevacizumab, 8.85 mg/mLtrehalose, 1.0 mg/mL L-leucine and 0.05 mg/mL pH 6.3 phosphate buffer. The spray solutions were spray dried in a pre-heated spray dryer at a solution feed rate of 10 g/min, an inlet temperature of 95-105° C., outlet temperature of 50° C. and a drying gas flow rate of 550 g/min. The solution was atomized through a two-fluid nozzle (1650/64) at a pressure of 25 psi. The spray dried particles were collected by a 2″ cyclonic separator.

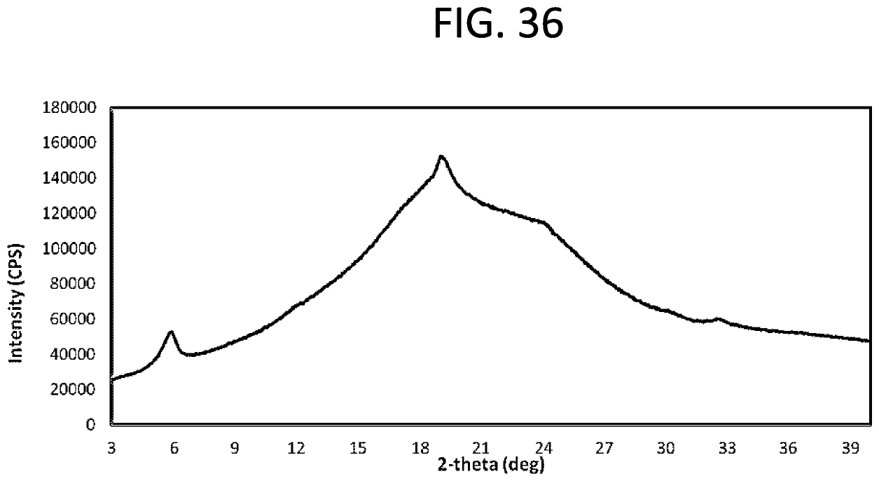

The L-leucine in the SDD was predominantly amorphous by PXRD, with only broad, weak L-leucine peaks present, shown in FIG. 36. The morphology via SEM was primarily spherical particles. The fine particle fraction normalized by the capsule fill mass measured using a Fast Scanning Impactor was 51±4.2 wt %.

The material was 6.0 wt % water measured by Karl Fisher titration, and potency measured by A280 absorbance was 4.4% bevacizumab on a dry basis.

### Example 24. Preparation of SDDs Comprising 40 wt % Bevacizumab/44.9 wt % Trehalose/10 wt % L-Leucine/5.1 wt % Phosphate Buffer

A spray solution was prepared containing 4 mg/mL bevacizumab, 4.49 mg/mL trehalose, 1.0 mg/mL L-leucine and 0.51 mg/mL pH 6.3 phosphate buffer. The spray solutions were spray dried in a pre-heated spray dryer at a solution feed rate of 10 g/min, an inlet temperature of 95-105° C., outlet temperature of 50° C. and a drying gas flow rate of 550 g/min. The solution was atomized through a two-fluid nozzle (1650/64) at a pressure of 25 psi. The spray dried particles were collected by a 2″ cyclonic separator.

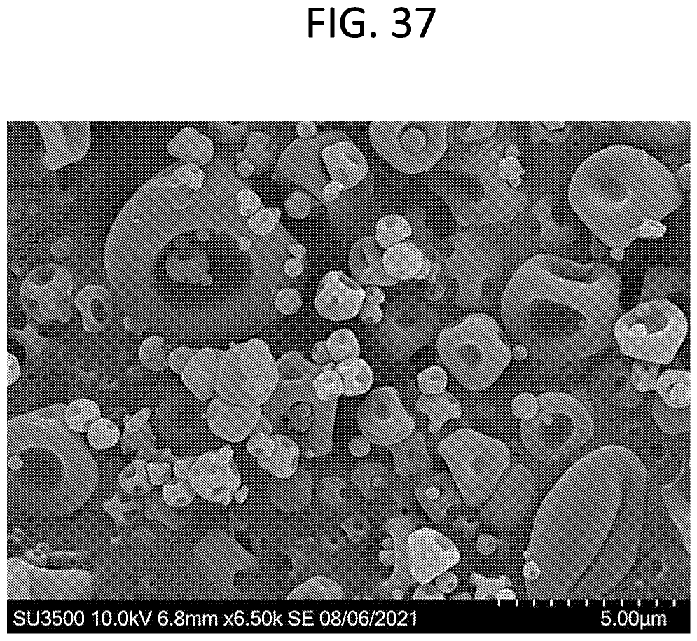

The SDD was amorphous by PXRD. The morphology via SEM is shown in FIG. 37, consisting of smooth, lightly collapsed particles. The fine particle fraction normalized by the capsule fill mass measured using a Fast Scanning Impactor was 43±5.6 wt %. The material was 6.3 wt % water measured by Karl Fisher titration, and potency measured by A280 absorbance was 41.3% bevacizumab on a dry basis.

### Example 25. Preparation of SDDs Comprising 40 wt % Bevacizumab/40 wt % Trehalose/20 wt % L-Leucine at Elevated Outlet Temperature 65° C.

A spray solution was prepared containing 4 mg/mL bevacizumab, 4 mg/mL trehalose, 2 mg/mL L-leucine in 1 mM pH 6.3 phosphate buffer. The spray solutions were spray dried in a pre-heated spray dryer at a solution feed rate of 10 g/min, an inlet temperature of 105-115° C., outlet temperature of 65° C. and a drying gas flow rate of 550 g/min. The solution was atomized through a two-fluid nozzle (1650/64) at a pressure of 25 psi. The spray dried particles were collected by a 2″ cyclonic separator.

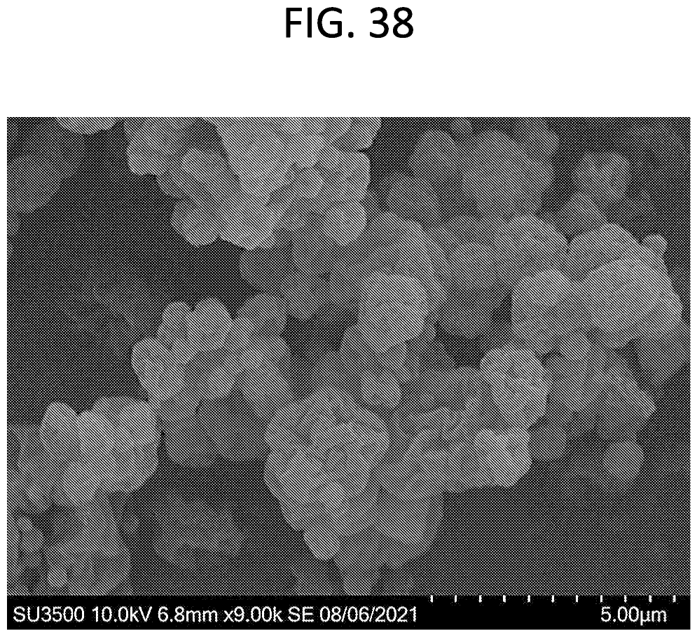

The L-leucine in the SDD was crystalline by PXRD. The morphology via SEM is shown in FIG. 38. When reconstituted in buffer at 20 mg/mL, dynamic light scattering measurements showed substantial presence of 100-1000 nm aggregates in the sample. This indicates that the mAb is partly degraded, leading to increased aggregation. The material was 4.1 wt % water measured by Karl Fisher titration, and potency measured by A280 absorbance was 33.9% bevacizumab on a dry basis.

### Example 26. Preparation of SDDs Comprising 40 wt % Bevacizumab/40 wt % Trehalose/20 wt % L-Leucine at Elevated Outlet Temperature 70° C.

A spray solution was prepared containing 8 mg/mL bevacizumab, 8 mg/mL trehalose, 4 mg/mL L-leucine in 1 mM pH 6.3 phosphate buffer. The spray solutions were spray dried in a pre-heated spray dryer at a solution feed rate of 10 g/min, an inlet temperature of 110-120° C., outlet temperature of 70° C. and a drying gas flow rate of 550 g/min. The solution was atomized through a two-fluid nozzle (1650/64) at a pressure of 25 psi. The spray dried particles were collected by a 2″ cyclonic separator.

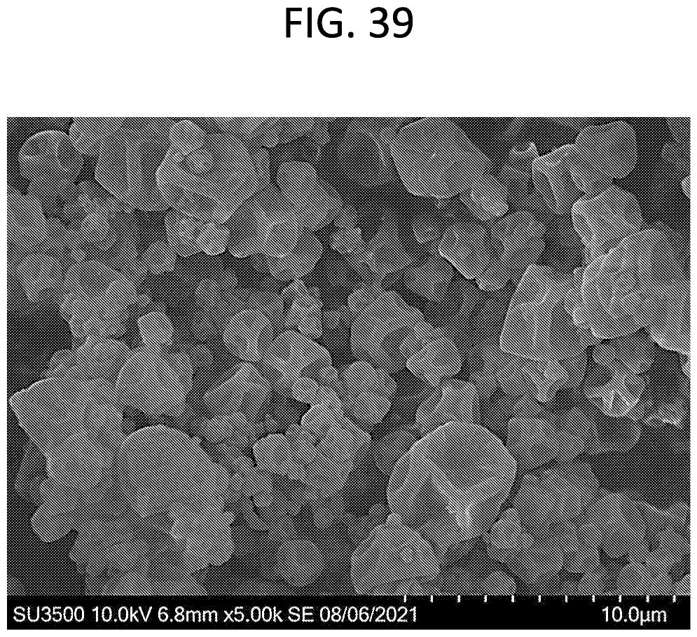

The L-leucine in the SDD was crystalline by PXRD. The morphology via SEM is shown in FIG. 39. When reconstituted in buffer at 20 mg/mL, dynamic light scattering measurements showed substantial presence of 100-1000 nm aggregates in the sample. This indicates that the mAb is partly degraded, leading to increased aggregation. The material was 4.7 wt % water measured by Karl Fisher titration, and potency measured by A280 absorbance was 39.8% bevacizumab on a dry basis.

1. A dry powder formulation suitable for administration via inhalation comprising:
1 wt % to 90 wt % of one or more antibodies or one or more angiogenesis inhibitors; and
a small molecular active pharmaceutical ingredient (API).

2. The dry powder formulation according to claim 1, wherein the angiogenesis inhibitor is a VEGF inhibitor.

3. The dry powder formulation according to claim 1, wherein the angiogenesis inhibitor is aflibercept, axitinib, bevacizumab, cabozantinib, lenvatinib, pazopanib, ponatinib, ramucirumab, ranibizumab, regorafenib, sorafenib, sunitinib, or vandetanib.

4. The dry powder formulation according to claim 1, wherein the angiogenesis inhibitor is bevacizumab, ramucirumab, or ranibizumab.

5. The dry powder formulation according to claim 1, wherein the antibody is benralizumab, dupilumab, lebrikizumab, mepolizumab, omalizumab, reslizumab, tralokinumab, oblitoxaximab, palivizumab, panobacumab, raxibacumab, atezolizumab, avelumab, balstilimab, bevacizumab, camrelizumab, cemiplimab, cetuximab, dostarlimab, durvalumab, necitumumab, nimotuzumab, nivolumab, panitumumab, pembrolizumab, prolgolimab, racotumomab, ramucirumab, ranibizumab, retifanlimab, sintilimab, tislelizumab, toripalimab, or any combination thereof.

6. (canceled)

7. The dry powder formulation according to claim 1, wherein the formulation further comprises a stabilizer, a dispersant, a buffer, or any combination thereof.

8. The dry powder formulation according to claim 7, wherein the stabilizer is trehalose, mannitol, raffinose, α-cyclodextrin, β-cyclodextrin, γ-cyclodextrin, inulin, pullulan, or any mixture thereof.

9-10. (canceled)

11. The dry powder formulation according to claim 7, wherein the dispersant L-leucine, tri-leucine, L-isoleucine, arginine, histidine, glycine, or any mixture thereof.

12-13. (canceled)

14. The dry powder formulation according to claim 7, wherein the buffer is phosphate, tris(hydroxymethyl)aminomethane (TRIS), acetate, glycine, citric acid, carbonate, or any mixture thereof.

15-21. (canceled)

22. The dry powder formulation according to claim 1, wherein the small molecular API is cisplatin, carboplatin, topotecan, paclitaxel, erlotinib, or any combination thereof.

23. The dry powder formulation according to claim 1, wherein the formulation comprises 1 wt % to 50 wt % of the angiogenesis inhibitor and 1 wt % to 50 wt % of the small molecular API, and the formulation further comprises 10 wt % to 88 wt % of a stabilizer, 5 wt % to 30 wt % of a dispersant, and optionally up to 5 wt % of a buffer, wherein the overall sum of concentrations of ingredients does not exceed 100 wt %.

24. The dry powder formulation according to claim 1, wherein the formulation comprises 5 wt % to 40 wt % of bevacizumab, 20 wt % to 80 wt % of trehalose, 10 wt % to 25 wt % of L-leucine, and 5 wt % to 40 wt % of the small molecular API, wherein the small molecular API is selected from cisplatin, carboplatin, topotecan, paclitaxel, or erlotinib, and wherein the overall sum of concentrations of ingredients does not exceed 100 wt %.

25. The dry powder formulation according to claim 1, wherein the formulation is a spray dried solid dispersion.

26. The dry powder formulation according to claim 1, wherein the formulation is a spray dried solid dispersion with a particle size distribution of:
(i) d90<50 μm; or
(ii) d50<5 μm; or
(iii) d10>100 nm; or
(iv) any combination of two or more of (i), (ii), and (iii).

27. (canceled)

28. The dry powder formulation according to claim 1, wherein the formulation is a spray dried solid dispersion with a particle size distribution of d90<10 μm, d50<3 μm, and d10>500 nm.

29. A capsule, a blister pack, or a blister strip, comprising a dry powder formulation according to claim 1.

30. (canceled)

31. A kit comprising a dry powder inhaler and one or more capsules according to claim 29.

32-33. (canceled)

34. A spray drying process suitable to manufacture a dry powder formulation according to claim 7, wherein the process comprises:
a) preparing a spray drying solution by dissolution of the angiogenesis inhibitor, the small molecular API, the stabilizer, the dispersant and optionally further ingredients in a spray drying solvent;
b) directing a drying gas at a particular inlet temperature at a particular drying gas flow rate into a drying chamber;
c) directing the spray drying solution at a particular liquid flow rate through an atomizing nozzle into the drying chamber, the drying gas exiting the drying chamber at an outlet temperature.
d) collecting obtained particles.

35. A spray drying process suitable to manufacture a dry powder formulation according to claim 7, wherein the process comprises:
a1) preparing a first spray drying solution by dissolution of the small molecular API, an optional stabilizer, the dispersant and optionally further ingredients in a spray drying solvent;
a2) preparing a second spray drying solution by dissolution of the angiogenesis inhibitor, a stabilizer, a dispersant and optionally further ingredients in a spray drying solvent;
b) directing a drying gas at a particular inlet temperature at a particular drying gas flow rate into a drying chamber;
c1) directing the two spray drying solutions simultaneously at particular liquid flow rates through two separate two-fluid atomizing nozzles into the drying chamber, said drying gas exiting the drying chamber at an outlet temperature;
d) collecting obtained particles.

36-46. (canceled)

47. A method of treatment, prevention, delay of progression, and/or maintenance therapy of asthma, COPD, lung infections, cystic fibrosis, or lung cancer, wherein the method comprises administering the dry powder formulation according to claim 1 to a human being or animal.

48-51. (canceled)
